(19) United States
(12) Patent Application Publication
(10) Pub. No.: US 2021/0025809 A1
(43) Pub. Date: Jan. 28, 2021

(54) MONITORING A CLOSED WATER SYSTEM

(71) Applicant: Hevasure Ltd., Darley Dale, Matlock, Derbyshire (GB)

(72) Inventors: Stephen Munn, Bakewell, Derbyshire (GB); Phillip Munn, Wirksworth, Derbyshire (GB); Ahmad Kharaz, Derby, Derbyshire (GB)

(73) Assignee: Hevasure Ltd., Darley Dale, Matlock, Derbyshire (DE)

(21) Appl. No.: 17/042,367

(22) PCT Filed: Mar. 19, 2019
(86) PCT No.: PCT/GB2019/050771

§ 371 Date: Sep. 28, 2020

(30) Foreign Application Priority Data
Mar. 27, 2018 (GB) 1804940.3
Mar. 27, 2018 (GB) 1804941.1
Mar. 27, 2018 (GB) 1804942.9
Dec. 13, 2018 (GB) 1820311.7

(51) Int. Cl. G01N 17/02; G01N 17/00; G01N 17/04; F24D 19/10

(52) U.S. Cl. CPC G01N 17/02; F24D 19/1051; G01N 17/04; G01N 17/004

(57) ABSTRACT

The present invention relates to a system and method for continuous monitoring of system health in closed water systems. Sensors are provided to measure a plurality of system parameters. The measurements are compared to threshold ranges. A diagnosis of system health, specifically in relation to corrosion, is derived from the comparison of at least a first parameter to its threshold range. The diagnosis is further refined by comparison of a further parameter to its threshold range. Also disclosed are example sensors and methods, including galvanic sensors, optical corrosion sensors and methods for monitoring the effectiveness of inhibitors in the water system using conductivity measurements.

This invention relates to methods and apparatus for monitoring corrosion in a closed water system, particularly in the context of heating, ventilation, and air conditioning systems (HVAC).

The invention could also be applied to chilled water systems (CHW), or low temperature hot water systems (LTHW), or any water system with a fixed volume of water for circulation. It is important within the field of closed water systems to be aware of any corrosion-related problems within the system. Corrosion in water systems is a known problem which can lead to the system requiring costly maintenance, or replacement incurring large direct and indirect costs.

A major cause of corrosion in closed water systems is dissolved oxygen being reduced at a cathode and causing a corresponding oxidation at an anode. The oxidation causes metal loss (corrosion) of the anode. Typically, the cathode may be copper and the anode may be steel, although any case where two different metals with different nobilities are exposed to the fluid can be affected in this way. Also anodic and cathodic sites can form on the surface of the same metal due to small defects or localised environmental differences (e.g. differential aeration). Corrosion therefore occurs when oxygen is drawn into the closed water system. For example, air may be drawn in to the system if the pressure is too low. In another example, if the volume of water in the system decreases due to a leak, the system may be topped up with make-up water from outside the closed system, which may typically be aerated, bringing dissolved oxygen into the system.

Corrosion can be monitored by taking water samples and analysing for dissolved ions. BSRIA guidelines ‘Pre-commission Cleaning of Pipework Systems’ (BG29/2012) are provided to teach a person skilled in the art a manner of conducting pre-commission cleaning and consequently detect corrosion. The BSRIA guidelines state that “the success of pre-commission cleaning is inferred from water samples that are analysed for a range of parameters including, but not limited to, suspended solids, iron and bacteria”. The teaching of using water samples to detect particulates is not effective in cases where the insoluble solids adhere to metal surfaces.

BSRIA guidelines ‘Water Treatment for Closed Heating and Cooling Systems’ (BG50/2013) provides further advice on maintaining water systems with predominant emphasis on water sampling and analysis.

Water sampling requires costly on-site visits, and does not provide a typical or representative reading of the corrosion rates due to a small sample being taken at irregular intervals; instead giving a snapshot of the corrosion which has occurred since the last measurement. It also cannot detect the environmental factors which give cause for corrosion such as dissolved oxygen levels and water make-up flow rate. In addition, the sample itself may become aerated during the measurement process, which skews the data. Water samples typically include soluble corrosion products, which can be detected, but insoluble corrosion products will not generally form part of the water sample. This means that water sampling techniques cannot detect the full extent of the corrosion, and so miss important information. The results of water sampling may be open to misinterpretation due to the previously mentioned effects, and depend on complex factors such as geometry. These factors vary between different systems, so it is hard to draw nuanced information out of such measurements, and instead provide only a broad picture.

The actual cause of the problem (e.g. corrosion) cannot be identified using this water analysis alone. This means that the detected corrosion may be rectified in an inappropriate manner to tackle the root cause. In addition, while corrosion may be detected by analysis of corrosion products, this relies on corrosion already having taken place.

Corrosion in water systems is a known problem which can lead to the system requiring costly maintenance, or replacement incurring large direct and indirect costs. By way of example, closed water systems can include heating, ventilation, and air conditioning systems (HVAC), chilled water systems (CHW), and low temperature hot water systems (LTHW) and are characterised by the system water being retained in the system for a long time and circulating past the components of the system many times (fresh water is added in a top up or a flush scenario, but these are relatively rare). Other water systems such as through flow industrial process systems may be arranged that water only flows past a given point once, i.e. each part of the system receives fresh water.

A major cause of corrosion in water systems is dissolved oxygen present in the water of the water system. The dissolved oxygen is reduced at a cathode and causes a corresponding oxidation at an anode. The oxidation causes metal loss (corrosion) of the anode. Typically, the cathode may be copper and the anode may be steel, although any case where two different metals with different nobilities are exposed to the water within the water system can be affected in this way. Also anodic and cathodic sites can form on the surface of the same metal due to small defects or localised environmental differences (e.g. differential aeration).

Pinhole or pitting corrosion is distinguished from other forms of corrosion in that where other forms of corrosion cause a broadly uniform loss of thickness from exposed surfaces, pinhole corrosion is extremely localised and can result in deep cavities in relatively compact areas of e.g. pipes of a water system. These narrow pits can extend a significant way into walls of the system, in the time it takes other, uniform types of corrosion to remove only a thin layer from the outside of the system. Clearly a problem occurs when pitting pinhole becomes so deep that it penetrates entirely through a pipe wall (that is, it creates a hole all the way through the pipe wall), becoming a pinhole. In some other cases, the pitting may penetrate only most of the way through a pipe wall, with the system pressure causing the final rupture. Of course, it is not just pipes which suffer from the effects of pinhole corrosion, since other elements, such as valves, joints, pumps, etc. can all be damaged by pinhole corrosion causing leakage.

Pinhole or pitting corrosion is a particularly pernicious problem as failure modes can occur much quicker than with other corrosion methods, since far less material needs to be removed before a pinhole corrodes through a given thickness of metal. Moreover, traditional corrosion detection methods tend to rely on the actual amount of material lost, either directly by detecting weight loss or somewhat more indirectly by measuring e.g. galvanic currents, which are indicative of the number of metal atoms lost. Pinhole corrosion is hard to quantify by these methods, since the actual lost mass of metal is small, so it can be hard to measure, and even harder to correlate with a risk of system failure, because a risk assessment as to how close a pipe is to rupturing necessarily must take account of the small scale details of the pinhole corrosion such as the width of the pinholes. Without this information a simple measure of lost mass of metal is insufficient to determine the likelihood of imminent system rupture.

There is a clear need to be able to monitor the corrosion of metals (e.g. carbon steel, brass, copper, aluminium etc.) in water systems. Existing sensors are prohibitively expensive for commercial use in monitoring systems aimed at the commercial HVAC market, for example. A typical corrosion rate sensor capable of determining the corrosion rate of just one metal can be very expensive. Desired is a low-cost corrosion sensor capable of withstanding environmental conditions such as temperature and pressure found in commercial water systems. In particular, a sensor adapted to detect and monitor pinhole corrosion is particularly desirable.

The present disclosure aims to address at least some of these drawbacks.

The following is an example embodiment of the apparatus of the present disclosure: the apparatus comprises a system of at least three sensors configured to measure parameters relating to environmental conditions that may lead to corrosion of a closed water system, or parameters that provide an indication of a corrosion-related problem. The parameters measured are based on at least one of the following: pressure; make-up water flow rate; dissolved oxygen; cumulative dissolved oxygen; inhibitor dosing levels; biofilm accumulation; temperature; conductivity; galvanic current; cumulative galvanic current; crevice corrosion rate; and/or pH. The sensors are configured to determine values of the corresponding parameters. A processor is configured to receive values of the parameters from the respective sensors and compare these values to threshold ranges that are retrieved from memory.

The threshold ranges can be predetermined, for example calculated based on various aspects of the system, such as size; types of metals making up the pipework, valves, and other components; types of inhibitors which are envisaged for use in the system; normal operating temperature, etc. In other cases, the system may be calibrated on installation, with the appropriate threshold ranges being determined on the fly. In yet other examples, the threshold ranges may be updated “on the fly”, that is, taking sensor data and updating the threshold ranges for some or all of the parameters. The thresholds can even be monitored and adjusted remotely by a user, for example if it is found that the system triggers too easily, or to suppress alerts when a planned maintenance event is in progress and which is anticipated to cause a parameter to fall outside the threshold range.

For example, the processor receives determined values of dissolved oxygen concentration from a dissolved oxygen sensor. In one example, the dissolved oxygen values are above the threshold range. The processor is further configured to provide a diagnosis of a corrosion state based on this comparison. In this example, the dissolved oxygen is above the threshold range, which means that oxygen has entered the closed water system. Dissolved oxygen is the main driver of corrosion, and therefore the diagnosis may comprise that the corrosion state is a positive corrosion state, which means that corrosion has been detected, or conditions have been detected which make corrosion likely to occur in the future. The links between these parameters can be used to intelligently narrow down the root cause of a problem, and suggest a corrective action to avoid a problem to the system state such as corrosion.

Following this, the cause of the positive corrosion state can be identified by using data from another sensor. In this case, one possible cause of dissolved oxygen entering the system may be pressurisation problems leading to air being drawn into the system. The diagnosis of the corrosion state is then refined based on the comparison of at least one further parameter to the corresponding threshold range. For example, the values of pressure may be compared to the threshold range to detect pressurisation problems. Other parameters that may also cause an increase in dissolved oxygen may also be used, for example make-up water flow rate. In one case, the dissolved oxygen is above the threshold range and the pressure is below the threshold range. The diagnosis can be refined to suggest that the low pressure state is the cause of the high dissolved oxygen levels, which may ultimately lead to corrosion.

In some examples the system may output a warning message such as “Oxygen entering the system due to low pressure—corrosion is occurring. Check pressurisation unit” or an alarm can be triggered. In this way and as will be clear from the foregoing, the extent and rate of corrosion currently happening, or likely to happen in the future, can be determined for each of the metals which make up the closed water system. The message output, any alarm messages, and/or the sensor readings can be presented to a viewer on a laptop or other viewing device. This can be a remote device, so that the user need not be close to the sensors, or even the building in which the water system is housed. Other embodiments will be described in more detail below.

Disclosed herein is an apparatus for monitoring a plurality of parameters for detecting corrosion in a closed water system, the apparatus comprising: a first sensor configured to determine values of a first parameter selected from the plurality of parameters; a second sensor configured to determine values of a second parameter selected from the plurality of parameters, wherein the second parameter is different from the first parameter; a third sensor configured to determine values of a third parameter selected from the plurality of parameters, wherein the third parameter is different from each of the first and second parameters; a memory for storing a threshold range for each of the first, second, and third parameters; and a processor, configured to: receive values of the first, second and third parameters determined by the sensors; compare the received value of the first parameter to a threshold range for the first parameter stored in the memory; provide a diagnosis of a corrosion state based on the comparison of at least the received value of the first parameter to the threshold range for the first parameter; refine the diagnosis of the corrosion state based on the comparison of a further parameter to a corresponding threshold range stored in the memory, the further parameter being one of the second and third parameters; wherein each parameter of the plurality of parameters is based on at least one of the following: pressure; make-up water flow rate; dissolved oxygen; cumulative dissolved oxygen; inhibitor dosing levels; biofilm accumulation; temperature; conductivity; galvanic current; cumulative galvanic current; crevice corrosion rate; and/or pH.

The first, second, and third sensors are configured to determine values of first, second, and third parameters respectively, selected from the plurality of parameters. For example, the first sensor may be a dissolved oxygen sensor, measuring the dissolved oxygen concentration in the water of a closed water system such as a HVAC system. Each parameter of the plurality of parameters is based on at least one of the following: pressure; make-up water flow rate; dissolved oxygen; cumulative dissolved oxygen; inhibitor dosing levels; biofilm accumulation; temperature; conductivity; galvanic current; cumulative galvanic current; crevice corrosion rate; and/or pH.

Each of the first, second, and third sensors may provide values of a plurality of parameters, either by direct measurement or inference from other measurements. This may comprise combined measurements. For example, the first sensor may provide values of both the conductivity and inhibitor dosing levels. In this example, the first sensor is configured to measure both of these parameters. These parameters may be connected and the measurement of one may be used to infer the value of the other.

The first parameter may for example be based on pressure, and the further parameter may be based on dissolved oxygen. In some embodiments, a parameter may be directly measured, or in other embodiments a parameter may be determined through proxy means, and the parameter value inferred.

The first, second, and third parameters may be measured substantially continuously or periodically by the first, second, and third sensors, respectively. This data may be stored in memory, for example. This memory may be located locally or accessible from a remote location. In this manner, recent results of the further parameter can be used to refine the diagnosis, in addition to the current instantaneous value. In some cases, not all sensors are operating at once, and the second or third sensor may be turned on as a result of a diagnosis indicating a corrosion state, in order to obtain data of the further parameter selected from the second or third parameter. All data can be viewed on a remote device such as a laptop, computer, mobile telephone, tablet, etc. A dashboard view may be provided, in which each parameter is presented together, sometimes with a brief summary of e.g. historical behaviour, recommended thresholds, or a colour coded health indication. Additionally, graphs may be plotted from the data in real time, or to view historical behaviour. A reporting and/or alerting facility may also be provided. Since the data from the sensors can be measured continually in real time, and relates to the properties of the system while in operation, the accuracy of the data reporting is greatly improved when compared with other techniques, e.g. intermittent sampling. Moreover, the effect of various changes to the system can be tracked to determine if they are having the desired effect (or indeed any effect at all). For example, in some cases the effect of adding inhibitor can be monitored to determine whether enough inhibitor has been added that corrosion is inhibited sufficiently or in some cases entirely. Inhibitor can take time to become effective, typically on the timescale of 1 to 3 days (although this varies with system size, system metals, inhibitor type, etc.). This means that the availability of continuous monitoring in the present system is particularly advantageous to track the development of a corrosion situation with time as an inhibitor becomes effective.

This can help to ensure that the dosage is actually effective. For example, in some cases, the manufacturer's recommended dosage may not be correct for completely passivating the exposed metal surfaces in a system. Any discrepancy can occur for a number of reasons, such as differences in: testing conditions, system metals; system parameters, etc. between the user's system and the inhibitor manufacturer's test system. Where the suggested value is actually lower than needed for a given system, without ongoing continual monitoring this discrepancy could only conceivably be noticed at a periodic test, at which point irreversible damage may have occurred to the water system. At the other end of the scale, it may be that the system actually requires a lower inhibitor concentration than the recommended dosage. Once more, complete passivation at a lower dosage could only reliably be detected by this ongoing continuous monitoring. A user can therefore use less inhibitor and save costs directly from the moment they install of the system. There are also environmental advantages to using less inhibitor.

The first, second, and third parameters are linked to the system health in general. In particular, they are used to inform the diagnosis of a corrosion state. The diagnosis of a corrosion state is an assessment of corrosion-related problems in the system. For example, the corrosion state is affected by the presence of corrosion occurring in the system, and this may be detected, for example by a sensor of the type set out below in more detail. The corrosion state is also affected by factors that may lead to corrosion e.g. dissolved oxygen in the closed water system. Each of the parameters listed above can be used to inform the corrosion state of the system. The corrosion state may correspond to adverse conditions that may ultimately lead to corrosion. The corrosion state may be conditions that are a pre-requisite of corrosion. A corrosion state may also include states where the system is currently being corroded. Some parameters are linked to the corrosion state in a precautionary manner. For example, some parameters (e.g. pressure) can be used to indicate that there is a possibility of corrosion occurring, particularly if the issue is not attended to. Other parameters (e.g. dissolved oxygen) can be used to indicate that corrosion will occur in the immediate future, or might already be occurring. Other parameters (e.g. galvanic current) can be used to indicate or confirm that corrosion is already occurring and that any chemical inhibitors added to the system are not being effective (e.g. they are too dilute or simply are inappropriate for passivating the metals in the system). Some parameters may even give a measure of corrosion, for example the integral of the galvanic current with time gives an indication of the amount of corrosion that has occurred, for example a thickness of material lost from exposed metal surfaces in the system. The diagnosis of the corrosion state takes account of the measured parameters and their comparison to threshold ranges in assessing the corrosion state. If the determined value of a parameter indicates that corrosion is likely, or is already occurring, then this indicates a positive corrosion state. A positive corrosion state is where a corrosion-related problem has been identified as a result of analysis of the determined value of the parameter. A further parameter can be used to refine the diagnosis of the corrosion state in an attempt to identify the cause of the corrosion-related problem, and identify a solution to stop corrosion occurring, or prevent corrosion from occurring, depending on the circumstance. Sensors are used to measure relevant parameters and thereby determine the extent of corrosion of metals in the system.

In examples where the received value of the parameter is within the corresponding threshold range, the diagnosis comprises that the corrosion state is a null corrosion state. This means that a corrosion-related problem has not been identified. This may be followed by using further results of the same parameter to ensure it was not an anomalous result. In other cases, the null corrosion state may be confirmed with further parameters measured using further sensors. For example this may detect a positive corrosion state even in the case of a malfunctioning sensor providing an incorrect null corrosion state.

The system parameters listed may broadly be grouped into three categories: ‘system integrity’, ‘water characteristics’, and ‘corrosion’. However, these categories are given merely as a guide for explaining the nature of the parameter type, wherein each parameter is assigned a category that best describes its nature, and should not be taken as a definitive label. In some examples, the ‘system integrity’ group of parameters include measures used to monitor the system in the first instance of detecting a corrosion state that may lead to a problem with the system health such as corrosion. For example, this group may comprise: temperature, pressure, dissolved oxygen concentration (instantaneous or cumulative), and make-up water flow rate. Optionally, this group of parameters may be thought of as primary parameters that are related to an initial stage in the process of detecting the possibility of corrosion before it has taken place e.g. the system may become aerated. Some measurements are better performed if the temperature of the system is kept within the threshold range at all times. For example, corrosion rates typically increase with temperature. A detection of high pressure can be used as an initial indicator of a corrosion state as this can lead to aerated make-up water being drawn into the system to compensate for consequential leaks, or low pressure can lead to air being drawn into the system at the highest point. A sudden change in conductivity could also be indicative of a leak. Both scenarios lead to an increase in dissolved oxygen in the system, thereby increasing the possibility of corrosion. A positive pressure is imperative at all times to prevent air ingress, especially at the highest point in the system. However, the pressure should not be raised too high such that water is lost through automatic air vents (AAVs) or pressure relief valves (PRVs) and consequently aerated make-up water is drawn in to the system to compensate, bringing with it dissolved oxygen.

Dissolved oxygen is an indication of a potential corrosive state as it is the main driver for corrosion. This indicates whether the system is air tight, and whether oxygen has entered the system with the potential to be reduced at cathodic sites leading to the corrosion of anodic sites. The dissolved oxygen concentration can be measured by a dissolved oxygen sensor

Although in some cases a sensor may determine instantaneous values of dissolved oxygen, for example periodically every hour, in some cases the parameter may be the cumulative dissolved oxygen. For example this may be calculated by integrating dissolved oxygen over time (e.g. expressed in PPM˜day). This can be useful to have these parameters separately or in combination, as the instantaneous dissolved oxygen can be a useful near-instant indication of a serious malfunction, while the cumulative dissolved oxygen will be indicative of ongoing problems that may not be picked up by a system monitoring spikes in instantaneous dissolved oxygen. This is important as a large amount of dissolved oxygen in the system for a short amount of time may be as equally as damaging as a small amount of dissolved oxygen for a long time.

It has been found that the integral of dissolved oxygen over time is a good indicator of total system damage caused by oxygenated water in the system. For the purposes of e.g. determining whether a warranty remains valid, the cumulative dissolved oxygen measure is shown to be a good indicator. The present system and method can be used to determine the ongoing cumulative dissolved oxygen and issue alerts when it reaches the warranty threshold. It can help users to intuitively see the damage accumulating, both where a high level is present for a short time (e.g. planned maintenance events such as flushing) and where a low level is present for longer periods.

The make-up water flow rate itself can be used as an indication of a potential positive corrosion state as fresh water entering the system, which is drawn in to compensate for losses, is a source of aeration. Other circumstances of make-up water being drawn into the system include if there is a leak or drain down of the system, and the building management system detects a pressure drop and causes fresh make-up water to enter the system. Monitoring the make-up water flow rate provides data on when and how much water is entering the system. Also, by monitoring the make-up water flow rate it is possible to check for leaks in the system, which would cause issues not only from a corrosion perspective but also to the usability of a building.

The ‘water characteristics’ group may, for example, comprise: conductivity, inhibitor dosing levels, pH, and biofilm. In some examples, this group of parameters may be thought of as secondary parameters that are related to a stage of detecting corrosion as it may begin to occur. The conductivity is related to corrosion because it provides information on the total dissolved solids (for example corrosion products) as well as inhibitor levels. The inhibitor dosing levels can be derived from conductivity measurements after removal of temperature effects, and can be used to determine the concentration of chemical corrosion inhibitors within the system. The effectiveness of the inhibitor can also be measured by using a sensor of the type set out below in more detail. This process is described in more detail below. Clearly the level of inhibitor has a strong correlation with corrosion within the system since it determines whether or not metal surfaces are adequately passivated in aerated water conditions. Measurement of pH can be used to indicate the potential for corrosion as it needs to be within a certain range depending on the type of metal. For instance, aluminium is likely to corrode if the pH of the system water is above 8.5. A determination of biofilm accumulation can be used to observe the presence of harmful microorganisms which cause microbial influenced corrosion (MIC) in metals. When biofilms form they can harbour these microorganisms such as sulphite reducing bacteria (SRB) which can lead to localised corrosion. Early detection is crucial to enable effective intervention such as with biocide treatment. Biofilms also can reduce the efficiency of a system by interfering with water flows (blockages) and heat transfer and therefore early detection is important.

The ‘corrosion’ group of parameters may, for example, comprise: galvanic current (cumulative or instantaneous), and crevice corrosion. Optionally, this group of parameters may be thought of as tertiary parameters that are related to a final stage in detecting corrosion and may often be used to confirm corrosion is taking place. Galvanic currents may be used to directly measure the level of corrosion by indicating when dissolution of e.g. steel surfaces is occurring and when inhibitors are ineffective at preventing corrosion in aerated water conditions. Crevice corrosion rates may also be monitored to further detect corrosion. This can occur in tight spaces such as weld seams and crimped joints when localised corrosion cells are established due to differential aeration or other environmental factors.

A processor is provided, configured to receive values of the parameters from the corresponding sensors, and to compare the received values to corresponding threshold ranges. The corresponding sensor may refer to the first, second, or third sensor. For example, the processor is configured to receive a value of the first parameter determined by the corresponding sensor, in this case the first sensor. Similarly, for the second or third parameter, the corresponding sensor will be the second or third sensor respectively. The information relating to the determining the value of each parameter is transferred to the processor. In one embodiment, an electrical connection is provided between each sensor and the processor, in order to allow for data transfer. In some cases, the processor may be configured to control the sensors.

The processor is also configured to compare the received value of the parameter to the corresponding threshold range. For example, the processor is configured to compare the received value of the first parameter to the corresponding threshold range (in this case the threshold range for the first parameter). In some cases the processor is configured to calculate a cumulative parameter e.g. cumulative dissolved oxygen or cumulative galvanic current by integrating the determined values of the parameter over time.

In some examples, the processor may comprise a data acquisition system. The processor may be a single processor, or it may exist as a plurality of processors. For example, it may be a set of processors which be present in a single location, or may be distributed. For example, information may be transmitted to a remote location and processed elsewhere, for example over the internet and processed on a remote PC or server. In this manner, the processor may be located remotely. For example, the comparison of the data to the threshold range may be performed at a remote location.

The threshold range depends on the parameter being measured, wherein there is a threshold range of acceptable values for each parameter. For example, for a given parameter such as pressure, the threshold range corresponds to a range of acceptable values, for example [X−Y] for which the system state may be under normal operating conditions, where X is the lower limit of the threshold range, and Y is the upper limit.

The threshold range corresponds to a corrosion state which relates to the system health or likelihood of threat to the health of the system e.g. corrosion. If the parameter is outside the threshold range, this may cause problems to the corrosion state and hence system health (e.g. persistence may lead to corrosion of metal surfaces within the closed water system). Therefore, a parameter value outside the threshold range is indicative of a positive corrosion state. The threshold range corresponds to a range of values such that if the parameter lies within this range, the parameter is at an allowable and normal operating value.

The threshold ranges are usually pre-determined and specific to each particular heating or chilled water system being monitored (e.g. chemicals being used will determine the acceptable levels for conductivity). In other embodiments, the threshold ranges may be updated remotely or automatically by the system, or determined in situ once the monitoring system has been installed.

A memory for storing a threshold range for each of the first, second, and third parameters is provided. This memory may be in the form of storage such as a hard drive, USB drive, or CD-ROM. In some cases this may be easily accessible by loading into random-access memory (RAM). The processor and the memory may form part of the same unit, or they may be separate entities. The memory may exist locally, or it may be accessed from a remote location. For example, the measurements of parameters may be transmitted to a remote location where it is stored in memory at a remote location. The memory to store the measurement of each parameter may also exist as a separate entity to the memory that stores the threshold ranges for each parameter, or they may be combined. Both of these memory units may exist locally and/or remotely. For example, the measured data may be stored locally on a data logger or SD card, and then transmitted to a remote server (e.g. cloud) for storage, where it may for example be viewable by a device such as a laptop or mobile phone. The threshold ranges stored in memory may be easily editable by the user, and may be changed remotely by a user as required.

The memory may store multiple ranges for each parameter. For example, for each parameter (e.g. pressure) a first threshold range may be stored for acceptable values of the parameter, wherein values outside the threshold range may cause corrosion-related problems and cause a positive corrosion state. An emergency threshold range may be stored for the same parameter, wherein values outside this threshold range may cause more immediate problems to the system health, which may comprise corrosion-related issues. For example, if the pressure increases to Z>Y, it may cause an emergency situation which requires different action to the pressure increasing only to Y. In other examples, it may cause other problems such as leading to a burst pipe or a safety issue. This emergency threshold range would typically have an emergency threshold upper limit greater than the normal threshold upper limit, and/or an emergency threshold lower limit lower than the normal threshold lower limit.

In another example, a different threshold range may be provided such that an acceptable range of values is provided during a planned maintenance event. For example, if it is known that a parameter will increase to a certain level during a cleaning event, this threshold range can be provided to ensure that the parameter value does not deviate from that expected. Maintenance events are described in more detail below.

Optionally, the threshold ranges for the first and further parameters correspond to a normal operating level of the corresponding parameter. If the parameter is within the threshold range, then the system behaves as normal. This may correspond to a safe or acceptable operating level, for example one which is not expected to cause damage to the system over long or short periods of time. This may also correspond to a null corrosion state. In some embodiments a system summary may be provided, such as at the end of each day, providing an overview of the corrosion state. In this case, null corrosion states may be used to inform the user that the system is behaving as expected with all measured parameters remaining within their threshold ranges.

The threshold range comprises an upper and/or a lower limit. In one example, the threshold range for pressure comprises an upper acceptable limit and a lower acceptable limit, wherein a pressure above the upper limit is outside the threshold range, and a pressure below the lower limit is outside the threshold range. For example if the threshold range for pressure is [X−Y], and a pressure of A (such that X<A<Y) is measured, then a comparison of this value to the threshold range will indicate that the pressure is within the allowable and normal operating range. However, if the measurement of pressure is B (such that B<X or B>Y), then a comparison of this value to the threshold range will indicate that the pressure is outside of the allowable and normal operating range, and indicative of a positive corrosion state

In another example, the threshold range for dissolved oxygen concentration comprises an upper limit, wherein a dissolved oxygen level above the upper limit is outside the threshold range, and the lower limit of the threshold range extends to zero, reflecting that it is not possible to trigger a positive corrosion state due to dissolved oxygen being too low. In some cases, negative values of certain parameters may be used in a diagnosis of a corrosion state, which is reflective of a sensor malfunction. In some examples, a sensor malfunction alert may also be outputted.

The values of the second and third parameters may be compared with threshold ranges corresponding to the second and third parameter respectively in a manner analogous to the determining a value and comparison of the first parameter.

For example, in the case of a value of pressure being received, there is a threshold range such that a normal operating range of pressures is defined. The comparison to the threshold range can be a measure of system health such as likelihood of corrosion, and can be used to provide a diagnosis of a corrosion state. If the value of the pressure is outside of the threshold range, for example the pressure is below the lowest value of the normal operating range, this may cause problems such as air being drawn into the system. This can have significant effects because air drawn into the system brings oxygen into the system. An increase in dissolved oxygen within the water of a closed water system can lead to an increased rate of corrosion. Therefore, the diagnosis may comprise a positive corrosion state.

The corrosion state may correspond to a single parameter being inside or outside the corresponding threshold range, or it may correspond to a plurality of parameters compared with their respective threshold ranges, combined together to provide a holistic overview of system health. Moreover, combining a plurality of sensors enables the cause and effect of corrosion-related problems to be identified, as is described in more detail below. Optionally, where a first and further parameter are determined independently, each providing a diagnosis of a corrosion state, the two measurements may be combined together to provide an overall holistic system state.

Below is a table showing example threshold ranges for various parameters.

| | Typical lower | | Typical upper | | System |
|---|---|---|---|---|---|
| Parameter | limit | | limit | | dependent? |
| Dissolved oxygen | None | | 0.2 | mg/L | No |
| Pressure | 1 | bar | 6 | bar | Yes |
| Water make-up flow | None | | 10 | L | No |
| Temperature | System dependent | | System dependent | | Yes |
| Conductivity | 1500 | μS/cm | 3000 | μS/cm | Yes |

| Inhibitor dosing | 80% nominal | 120% nominal | | Yes |
|---|---|---|---|---|
| level | | | | |
| pH | 6.5 | 8.5 | | Yes |
| Galvanic currents | None | 0.3 | mA | No |
| Crevice Corrosion | none | 10 | Ω | No |
| (resistance of metals | | | | |
| wire) | | | | |

Some parameters are dependent on the system, for example the pressure depends on the system, the arrangement of pipework, and where in the system the pressure is measured. The typical range of values indicates a safe operating range. For example, a pH between 6.5 and 8.5 indicates a normal system. However, if the pH deviates from this range, this could be indicative of a positive corrosion state. For example, above 10 L the water make-up flow indicates a large leak may have occurred.

The processor is configured to provide a diagnosis of a corrosion state based on the comparison of at least the received value of the first parameter to the threshold range for the first parameter.

Optionally, the processor is further configured to provide an indication of normal system health in the event that the value of the first parameter is within the threshold range for the first parameter. This corresponds to a null corrosion state, and may be followed up with analysis of further parameters to determine whether other corrosion states (i.e. having different causes, expected futures, etc.) are present in the system. This may be included in the diagnosis. Furthermore, the diagnosis corresponds to a corrosion state for that parameter. For example, if the pressure is measured, and upon comparison to the threshold range of acceptable pressures, the pressure value is determined to be within the threshold range, then the diagnosis may indicate that the corrosion state is null. However, this result may be combined with others in order to confirm that the system state is normal. In the example above, this may correspond to a low risk of the measured pressure causing corrosion-related problems.

The diagnosis provided by the processor may further comprise information about the parameter values within the threshold range e.g. the pressure is A, which is within the threshold range [X−Y]. It may further comprise a warning that the parameter is close to an upper or lower limit of the threshold range, which may comprise, for example, that although the measured pressure is within the threshold range, it is at the high end of the range, and the pressurisation unit may need attending to in order to prevent the pressure consequently moving outside the threshold range. For example, in some embodiments such states may be triggered when parameters reach a certain percentage of the threshold limit. In some cases a series of measurements of the same parameter over time may be used to extrapolate the parameter over time, to assist in determining if (and even in some cases, when) the parameter is expected to depart from the threshold range.

In other examples, analysis of changes to parameter values within the threshold range may be considered. For example, if a parameter rapidly changes but is still within the threshold range, this could be indicative of a positive corrosion state, and can be an early warning and used as a preventative measure before the threshold range is exceeded. For example, the gradient of a parameter value with time may be calculated, and if the gradient exceeds a certain value, for example over a certain interval of time, this can be used to inform the corrosion state. In some cases this information can be used to predict when the value is likely to exceed the threshold limit. A parameter may be deemed to be outside its threshold range even when the instantaneous value is within the threshold range if, for example, the gradient of that parameter with respect to time predicts that the parameter value will be outside of the threshold range within a predetermined amount of time, e.g. 4 to 12 hours where measurements are taken, for example, every 10-30 minutes, although other sampling rates are possible.

Optionally, the processor is further configured to provide an indication of a positive corrosion state in the event that the value of the first parameter is outside the threshold range for the first parameter. As a result of a parameter being outside the acceptable threshold range, the positive corrosion state is an unfavourable condition that is linked to the corrosion of the system. This may be included in the diagnosis. For example, the positive corrosion state may correspond to an imminent corrosion event unless corrective action is taken. The diagnosis provided by the processor may comprise information corresponding to the comparison of the value of the first parameter to the threshold range for the first parameter. In some embodiments it may further comprise an indication that the value of the first parameter is outside the threshold range. For example, if the first parameter measured is the system pressure, and the comparison of the pressure to the threshold range determines that the pressure is lower than the lower limit (or indeed higher than the upper limit) of the corresponding threshold range, then the diagnosis may provide information that the pressure is too low (or equivalently, too high). The diagnosis will indicate a positive corrosion state in this instance, where the pressure being outside the threshold range may lead to corrosion-related problems. The diagnosis of a corrosion state may vary. For example, the diagnosis of a positive corrosion state may comprise different severities based on the threat of corrosion e.g. whether corrosion has already been detected, or whether corrosion may occur in the future. Additionally, such a diagnosis may include information relating to how the diagnosis was arrived at. For example: the parameter used to arrive at the diagnosis; historical behaviour of that parameter; and the exact value of the parameter can all help to narrow down the potential causes of the positive corrosion state. Where the parameter which leads to the diagnosis is low pressure, for example, the cause may be air drawn into the system. The magnitude and historical behaviour of the pressure can help to determine the magnitude of the air intake, whether it is worsening, etc. This in turn can lead to an estimation of how much dissolved oxygen has entered the system, and how fast it is currently entering the system.

The diagnosis comprises a comparison of the determined value of the parameter with the corresponding threshold range. In some examples, the comparison comprises that the measured parameter lies within or outside the threshold range. In some examples, the comparison further comprises an indication of the position of the parameter value within or outside of the range, e.g. whether it is close to the upper or lower limit of the threshold range, whether it is close to the average expected value, or whether is far exceeds the upper or lower limit. In some examples it may comprise information relating to the gradient of the parameter values with respect to time, how quickly it is changing, and in some cases a prediction of when it will exceed the threshold limit. In some examples, this comparison comprises details of the numerical measured pressure values compared to the threshold range. In some examples, the diagnosis comprises the identification of the problem e.g. the dissolved oxygen concentration is above the upper limit of the threshold range, and that air or make up water has somehow been drawn into the system.

Optionally, in the event of a positive corrosion state, the processor is further configured to refine the diagnosis to provide an assessment of the potential causes of the positive corrosion state. This may be included in the diagnosis. In this case, the positive corrosion state corresponds to the determined valued of the parameter being outside of the threshold range. For example, if the measured pressure is too low, the cause will be a low pressure state of the system. In another example, if the measured dissolved oxygen concentration is too high, the cause may be that air has been drawn into the system, raising the dissolved oxygen concentration. This may be due to low pressure in the system or other factors. Conversely, if the measured dissolved oxygen concentration is too high, another cause may be that the pressure is too high, leading to water loss through automatic air vents, and consequently aerated make-up water has been drawn into the system. The diagnosis may suggest potential causes of the positive corrosion state based on the parameter value determined that lies outside of the threshold region.

Optionally, in the event of a positive corrosion state, the processor is further configured to provide an assessment of the threat to the system health as a consequence of the positive corrosion state. This may be included in the diagnosis. For example, if the assessment in the diagnosis is that the pressure is too low, this may have led to air being drawn in to the system, causing an increase in dissolved oxygen. This oxygen may lead to an increased rate of corrosion of metal surfaces within the closed water system, particularly of anodic sites which typically may be made of steel. In this example, the diagnosis may provide information that the low pressure may lead to corrosion if left unattended.

Following a threshold value being exceeded, interpretation is carried out to understand the reason for the value being outside the threshold range (i.e. the reason for a positive corrosion state). This interpretation may be performed by the processor. For example, logic tables may be used to perform these automatic interpretations. In some cases, the system may suggest possible causes and effects to the user, who in turn performs additional interpretation. In other cases, the interpretation may be performed by a human user studying the data from the sensors. In some cases, a human user may be alerted in the event of a threshold value being exceeded and informed that interpretation is necessary.

Optionally, in the event of a positive corrosion state, the processor is further configured to provide a suggested correction to rectify the positive corrosion state. This may be included in the diagnosis. In some examples this may be through a corrective action. For example, the diagnosis may provide information suggesting that the user should attend to the pressurisation unit to adjust the pressure back to within the threshold range of acceptable pressures for normal and safe operation.

The processor is further configured to refine the diagnosis of the corrosion state based on the comparison of a further parameter to the corresponding threshold range stored in the memory, the further parameter selected from the second and third parameters. The further parameter is either the second or the third parameter, wherein the parameter used is based at least in part on the diagnosis. For example, if the original diagnosis indicated a positive corrosion state, a further parameter can be used to validate the first parameter value, confirm the positive corrosion state or identify causes of the positive corrosion state or potential consequences of that state if left unchecked. The further parameter is usually one which is being measured continually or periodically in any case. For example, if the first parameter is dissolved oxygen, and the diagnosis comprises that the dissolved oxygen is above the upper limit of the threshold range, then the further parameter may be galvanic current which can be used to detect signs of corrosion and refine the positive corrosion state. This can be used to intelligently assess the system health and efficiently and effectively monitor signs of corrosion.

Some or all of the sensors may be operational throughout the method, and obtaining results. After the diagnosis, a relevant and related parameter is used to refine the diagnosis. For example, the apparatus may be configured to consider predetermined pairs or groups of parameters that are related to one another such that one parameter can help to determine the cause of a positive corrosion state diagnosed from another parameter. The predetermined groups of parameters may be provided as part of the system operating protocols, e.g. stored in an on-board memory or the like. The parameter that is used may be related to the first parameter, such that the results of the further parameter can be used to refine the diagnosis of the corrosion state obtained on the basis of the measurement of the first parameter. In one example, all sensors are monitoring their respective parameters, and the results are stored in memory. The processor uses a further parameter which is related to the first parameter in order to refine the diagnosis of the positive corrosion state, and attempt to identify the cause of the positive corrosion state. In other cases, the processor may cause the further sensor to begin taking measurements of values of the further parameter as a result of the diagnosis of the corrosion state related to the first parameter.

Consider an example where the galvanic current was the first parameter and it was deemed to be above the threshold limit, indicating a positive corrosion state. The processor then uses a further parameter to refine the diagnosis and attempt to identify the cause of the positive corrosion state. The further parameter may for example be the dissolved oxygen concentration within the water system, which may be measured by a dissolved oxygen sensor. The processor is further configured to receive a value of the further parameter determined by the corresponding sensor. The processor is further configured to compare the received value of the further parameter to the corresponding threshold range. For example, if the further parameter is the second parameter, then the processor receives the value from the second sensor, and then compares this value to the threshold range for the second parameter. If the further parameter is the third parameter, then the processor receives the value from the third sensor, and then compares this value to the threshold range for the third parameter. In this example, the measurement is compared to a threshold range of acceptable dissolved oxygen concentrations. The diagnosis is refined based at least on the comparison of the further parameter to the corresponding threshold range. If the value of the further parameter is outside the corresponding threshold range, then the diagnosis of a positive corrosion state may be confirmed, if this corresponds to the expected result of the positive corrosion state. However, if the measurement of the further parameter is within the corresponding threshold range, then the diagnosis may be corrected.

Many of the parameters may have a direct link with one or more other parameters. Correspondingly, a change in one parameter may cause an expected change in a linked parameter. For example, if the make-up water flow rate changes such that aerated water has entered the system, then the dissolved oxygen concentration is expected to rise due to the intake of oxygen into the system, and potentially the conductivity and galvanic current are expected to change if the problem is not attended to. The links between these parameters can be used to intelligently narrow down the root cause of a problem, and suggest a corrective action to avoid a problem to the system state such as corrosion. In this manner, the analysis of the further parameter allows the diagnosis to be refined if there is such a link between the two parameters. In one embodiment, the first parameter may be chosen from the ‘corrosion’ group and the further parameter may be chosen from the ‘system integrity’ or ‘water characteristic’, allowing the further parameter to provide an indication of the cause of the positive corrosion state.

Another example may involve the first parameter being chosen from an environmental parameter (e.g. pressure), and the further parameter being chosen from a more direct corrosion-related parameter (e.g. dissolved oxygen). In this manner, the system can identify if an environmental factor (pressure) has led to increased dissolved oxygen levels, which in turn may lead to corrosion. This allows the system to take preventative action before corrosion occurs by monitoring conditions that may eventually lead to corrosion, and attempt to rectify them before corrosion occurs.

If the first and further parameters are not linked, the system still provides a way of assessing the threat from two unrelated parameters. For example, a very coarse first pass can be implemented, which takes two very distantly related parameters, such as biofilm accumulation and dissolved oxygen concentration. The purpose in this case, is to assess whether corrosion from each of different sources is occurring. The results (both, neither, or just one of the two sources) can prompt a repeat of the method, focussing on appropriate parameters to ascertain the root causes of, and appropriate response to, any detected corrosion.

Optionally, the processor is further configured to provide a refined indication of a positive corrosion state in the event that the value of the first parameter is outside the first threshold range, and in the event that the value of the further parameter is outside the corresponding threshold range.

In one embodiment where the dissolved oxygen was measured as the first parameter and determined to be higher than the upper limit of the threshold range, a corresponding diagnosis of a positive corrosion state was consequently provided. The diagnosis may for example comprise as assessment of the potential consequences of the problem, which in this case may be that the oxygen drawn into the system may lead to corrosion. The diagnosis may for example comprise an assessment of the cause of the positive corrosion state. In this case, pressurisation problems may have caused an increase in dissolved oxygen concentration, or aerated make-up water has been drawn into the system.

In this case, the further parameter for refining the diagnosis may be pressure, and the measurement of the pressure is compared to the corresponding threshold range for pressure, then this would allow the diagnosis to be refined. For instance, if the measured pressure is lower than the lower limit of the threshold rage, then this information can be used to refine the diagnosis, confirming that the pressure is too low and that this may have led to oxygen being drawn into the system via automatic air vents (AAVs) or pressure relief valves (PRVs), confirming that low pressure has had adverse effects, raising dissolved oxygen levels that may lead to corrosion.

In this scenario, the original diagnosis would have comprised that the dissolved oxygen is too high and this may be caused by air being drawn in to the system. As a result of the measurement of the low pressure state below the threshold range, the diagnosis is refined to confirm that the cause of the positive corrosion state is low pressure. Having identified the cause of the problem, the pressurisation unit can be attended to, and further intake of air can be prevented.

However, if the measurement of the pressure as the further parameter is determined to be within the threshold range, then the diagnosis can be corrected. For example, the refined diagnosis provides information that the pressure is not outside the threshold range as expected and therefore is probably not the cause of the positive corrosion state. In this case, another parameter may be used to determine the cause of the increased dissolved oxygen levels e.g. make-up water flow rate. In some embodiments this may be further validated by additional measurements of the same or different parameters to either confirm or correct the diagnosis. The refined diagnosis may comprise a suggestion as to the next parameter to be assessed. In this case, the refined diagnosis may suggest that the make-up water flow rate is checked, suggesting this as the potential cause of the positive corrosion state.

In some cases, the parameters for determining whether a particular potential cause is in fact the cause of the positive corrosion state can be ranked in order of likelihood. The highest ranked positive cause can be taken to be the cause (or at least the main cause). In some cases, each parameter in the list may be considered as a potential cause, with each providing a null result. This may lead to a particular type of error message which alerts a trained professional to attend to take more in-depth measurements.

In another example, the first parameter may be dissolved oxygen, and the further parameter is galvanic current. If this dissolved oxygen is above the threshold, this may lead to corrosion. The galvanic current may be used to confirm if corrosion is occurring, and the diagnosis can then be refined.

Optionally, in the event of a positive corrosion state, the refined diagnosis comprises a refined assessment of the potential causes of the positive corrosion state, based on whether the further parameter is within or outside the corresponding threshold range. As such, the processor may be configured to provide a refined assessment of the potential cause of the positive corrosion state. In conjunction with the original diagnosis, the comparison of the further parameter value to the corresponding threshold range can be used in some cases to improve the diagnosis of the potential cause of the positive corrosion state, in the manner described above by example. For example, if the further parameter is outside the threshold, this is indicative of this being the cause, and if the further parameter is within the cause, this suggests that it is not the cause, and may be due to another factor. If the further parameter is close to the threshold, or very steeply changing, then the refined diagnosis may suggest that this may be the cause and further analysis is required, which may involve further monitoring of the same parameter, and perhaps the use of other parameters that might be possible causes.

The refined diagnosis may also comprise an improved identification of the cause of the problem. For example, if make-up water had been detected to be entering the system, the potential cause could be indicated as a water leak causing make-up water to be drawn into the system, or high pressure causing water loss through pressure relief valves or automatic air vents and hence causing make-up water to be drawn into the system. In this example, by using the pressure as a second parameter, the diagnosis can be refined to verify whether the pressure is within or outside the threshold range. For example, if the pressure is above the upper limit of the threshold range, then there may be a high pressure state which has caused water loss through the air vents or valves and led to make-up water being drawn into the system. Conversely, if the pressure is within the threshold range for pressure, then there may be a leak in the closed water system that needs attending to. The system may maintain which system parameters are related to others (e.g. pressure and make-up water flow rate in the previous example) in memory, and perform measurements of necessary parameters to confirm or correct a diagnosis in this way, or it may perform the measurements of all the parameters and use this information to infer a refined diagnosis. The diagnosis may then be correspondingly refined to convey the further improvements in the identification of the cause of the problem. This allows the cause of the problem to be targeted and easily rectified, without making a mistake in identifying the original cause of a problem.

Optionally, in the event of a positive corrosion state, the refined diagnosis comprises a refined assessment of the threat to the system health as a consequence of the positive corrosion state. The refined diagnosis based on the measurement of a further parameter can be used to: confirm that a threat to system health (e.g. corrosion) as a consequence of the positive corrosion state is likely; or correct the diagnosis based on the further parameter not being outside the threshold range. For example, the first parameter of pressure may be measured to be lower than the threshold range, but the further parameter of dissolved oxygen may be within the threshold range, meaning that the low pressure has not caused the expected positive corrosion state of increasing dissolved oxygen levels. In this case, the pressure may be measured again to determine if the first measurement was incorrect, or whether the effect has been delayed.

Optionally, in the event of a positive corrosion state, the refined diagnosis comprises a refined suggested correction to rectify the positive corrosion state. For example, if the refined diagnosis comprises that the temperature is too low, the suggested correction may include that the temperature should be increased by turning on a heater, boiler, or opening a valve to allow heating.

In some embodiments, further results of the first or further parameter may be used in order to refine the diagnosis. Consider an example where the first parameter is determined to be outside the threshold range, and the second parameter is determined to be within the threshold range. New values of the first parameter may be used. For example, if further values of the first parameter were within the threshold range, it may be that the first value was an anomalous result, wherein the anomaly can be recorded and the first parameter can be monitored closely to ensure it was incorrect. On the other hand, if a further value of the first parameter was received and deemed to be still outside the threshold range, it may be that the first value was valid but the effect has been delayed, which may explain why the value of the second parameter was not as expected.

For example, in a case where one of the values determined is deemed to have been anomalous, the diagnosis can be corrected, or in some scenarios may be cancelled. A preventative action may be taken if necessary, for example the pressurisation unit may be attended to in order to ensure that the system pressure is at the required level. In another example where the effect has been delayed, the diagnosis may comprise that the dissolved oxygen levels have not increased beyond the threshold range as expected, but they may have increased within the range, and may increase beyond the upper limit of the range in the future. In this case, preventative action can be taken before the oxygen levels increase out of the threshold range. For example if the dissolved oxygen levels do not increase as expected after a previously-measured decrease in pressure, the pressure may be measured again at a later stage, and also the dissolved oxygen levels may be measured at a later stage in an attempt to validate the refined diagnosis.

Previous systems that detect corrosion may lead to an inappropriate solution to a problem. The standard response to a corrosion problem is taken after detecting the after-effects of corrosion (e.g. metal ions detected in a water sample). This catastrophically relies on corrosion already taking place and causing damage to the system. A typical response is to add a chemical inhibitor to the water system in response to the detected corrosion. Not only does this allow e.g. leaks to continue, but inhibitor will continue to be added unnecessarily. A better solution to this specific situation is to repair the leak and ensure that less inhibitor is wasted, and costs saved. It will be apparent that other underlying causes can be beneficially addressed by the present disclosure prior to their causing excessive damage to the system. Chemical inhibitors can also help to stabilise the pH to within a desirable range as well as interfering with cathodic processes. Other chemicals may have a biocidal action to kill microbes.

Prior devices often provide an inappropriate solution because they do not necessarily solve the underlying problem. For example, if corrosion has been caused by a low pressure state in the water system, this may not be identified as the root cause due to a lack of combination of parameter measurements. The systems and/or methods of the present disclosure help to identify the underlying cause of the corrosion thereby allowing correction of the cause. The systems and/or methods of the present disclosure can also be used as a preventative method allowing a system health issue (e.g. a corrosion-related problem) to be identified early, in some cases even before it has taken place. For example, a low pressure state can be identified, and can be confirmed by an increase in dissolved oxygen concentration. The problem can be corrected before corrosion has taken place, preventing damage to the system, and saving the huge costs of repairs or replacement.

Optionally, the apparatus further comprises at least one additional sensor configured to determine values of an additional parameter selected from the plurality of parameters, the apparatus further comprising a corresponding threshold range for each additional parameter stored in the memory. The at least one additional sensor may be connected to the processor in an analogous manner to the first, second and third sensors.

Optionally, the processor is further configured to refine the diagnosis of the corrosion state based on the comparison of an additional parameter to the corresponding threshold range. In this case, the processor is configured to receive a value from at least one additional sensor.

For example, an additional parameter may be used to further refine the diagnosis of the corrosion state from the results of the first and further parameters, or may be used to confirm the effectiveness of a corrective action. Additional parameters may be used to refine a previously-provided diagnosis, which may improve the suspected cause of the corrosion state. In other cases it may be used for validating, confirming or correcting a diagnosis. Clearly a wide range of parameters can be used to provide a holistic overview of the entire system, providing a rigorous diagnosis of the system health such as likelihood of corrosion and an assessment of the corresponding cause.

Optionally, at least one of the additional parameters is different from the first, second, and third parameters. Optionally, at least one of the additional parameters is the same the first or further parameter. In some cases, each of the additional parameters is different to each of the first or further parameters. In some examples where the further parameter was the second parameter, the additional parameter may be the third parameter. In other examples where the further parameter was the third parameter, the additional parameter may be the second parameter.

Optionally, in the event of a positive corrosion state, the refined diagnosis comprises a refined assessment of the potential cause of the positive corrosion state, based on whether the additional parameter is within or outside the corresponding threshold range. The refined diagnosis for the additional parameter may be performed in an analogous manner for the further parameter. For example, the measurement of an additional parameter, or a plurality of additional parameters, may refine the diagnosis and narrow down the potential causes of the positive corrosion state.

Optionally, in the event of a positive corrosion state, the refined diagnosis comprises a refined suggested correction to rectify the positive corrosion state, based on whether the additional parameter is within or outside the corresponding threshold range.

Optionally, in the event that the value of the first or further parameter is outside the corresponding threshold range, refining the diagnosis comprises: confirming the diagnosis in the case that the value of the additional parameter is outside the corresponding threshold range; or correcting the diagnosis in the case that the value of the additional parameter is within the corresponding threshold range. In one embodiment, this may be used to observe how the measurement of one parameter changes over time. For example, if the pressure is below the threshold region in the first measurement, the second measurement of the same parameter can be used to validate the first measurement by either confirming it or correcting it. For example, if the second measurement of the same parameter maintains that the parameter is outside of the threshold range, then this confirms the first measurement and the corresponding diagnosis, and the system is aware that the problem is persisting over the time interval between the first and second measurement.

Equally, if the second measurement of the same parameter contradicts the first measurement, and the parameter is now inside the threshold range, then the system is aware that the problem no longer exists, and may correct the diagnosis. This may mean the first measurement was a false reading, potentially due to a fluctuation. Optionally, this could be further validated by a third measurement of the same parameter at a later time. This situation may also correspond to an instance in which corrective action has been taken between the two measurements, and the measurement taken after the corrective action can be used to confirm the success of the action. For example, if the first measurement determines the pressure is too low, and a diagnosis is provided, then the action taken may comprise attending to the pressurisation unit and increasing the pressure. The second measurement may comprise validating that the corrective action has been successful such that if the second measurement determines that the pressure is within the threshold range, then the corrective action has been successful. Conversely, if the pressure is not within the threshold range, the corrective action has not yet been successful. In this manner, the same parameter may be monitored continuously or discretely over time.

In some embodiments, at least one of the additional parameters may comprise other parameters than those listed above. It is expected that a person skilled in the art would understand that an additional parameter may comprise any parameter useful for indicating the system health of a closed water system, for example comprising an indication of potential corrosion within a closed water system.

The determination of parameter values may involve direct measurement of the parameter, or it may involve a further adjustment for example due to temperature effects. Some parameters may be affected by temperature. For example, temperature may have an effect on some parameters, meaning that readings are skewed. For example, a higher value may be given of a certain parameter if the temperature is higher. For example, it is known that conductivity is affected by temperature. In some examples, a rise in conductivity may be caused by a rise in temperature which may incorrectly indicate a corrosion-related issue or incorrectly state the severity of such a condition, unless corrected. These temperature effects may be corrected by calibrating values based on a known trend of the parameter with temperature. For example the effect of temperature may be stored in memory and used to calibrate each measurement to compensate for temperature effects. In some examples the parameter value can be automatically adjusted to compensate for temperature effects. In other examples the user may be alerted that a significant temperature change has occurred, and that results may be skewed.

In another example, temperature may have a direct consequence on a corrosion-related problem. For example, temperature may increase the rate of reaction (i.e. corrosion). For example, a certain level of dissolved oxygen may cause different corrosion rates at different temperatures. However, for an increased corrosion rate, dissolved oxygen is consumed at an increased rate which may therefore lead to the overall amount of corrosion being the same. Correspondingly, the user can be alerted to a change in temperature and the problems that this may cause. In some examples the temperature can be controlled to ensure that corrosion is minimised. This may be performed automatically, for example if the temperature is outside the threshold range. Examples of temperature correction are discussed in detail below in reference to the systems and methods for determining inhibitor levels.

Optionally, the apparatus is configurable in a corrosion detection mode or a maintenance mode, and wherein in the event that the apparatus is configured in the maintenance mode, the threshold ranges of the first and/or further parameters are adjusted to correspond to the expected values during a maintenance event. In other examples, the threshold range of other parameters different to the first or further parameters are also adjusted. In some cases, the threshold ranges of any additional parameters are also adjusted to correspond to the expected values during a maintenance event. The adjusted maintenance threshold ranges may be stored in memory for each of the plurality of parameters listed above.

The corrosion detection mode is the normal operating mode of the system that provides a diagnosis relating to the system health (e.g. detecting signs of corrosion) as described above.

The expected values of the threshold ranges may be adjusted depending on the maintenance event planned. The maintenance mode may be selected prior to any planned maintenance events occurring to the system. The maintenance mode may be used for logging planned maintenance events and ensuring they are implemented correctly, while using the same methods of the corrosion detection mode to prevent any deviation in parameters from an acceptable threshold range, which may be different from the threshold range of the corrosion detection mode based on expected changes to the parameter levels as the system undergoes a maintenance event. In some cases, switching the system to maintenance mode is performed manually by the user. In other cases, it is automatic. For example, this can be relied upon by manufacturers who want to ensure that maintenance events are carried out correctly by a user, for example for system warranty purposes.

Optionally, the processor is further configured to monitor a specific further parameter based on a specific maintenance event, for example a planned event, or an unplanned event. For example, when a certain planned maintenance event such as a mains water flush occurs, the further parameter may be chosen from certain parameters that are known to be affected by the maintenance event e.g. make-up water flow rate and/or dissolved oxygen. In some examples, the processor is configured to use specific first and further parameters based on a planned maintenance event. In other examples, additional parameters other than the first or further parameters are used based on a planned maintenance event.

Optionally, in the event that the apparatus is configured in the maintenance mode, the processor is further configured to indicate that at least one parameter is outside the corresponding threshold range but inside the corresponding adjusted maintenance threshold range. This can be used to inform the user that a planned maintenance event is proceeding as expected. For example, during a maintenance event such as a cleaning process, many sensors may measure parameter values that are outside the threshold range for normal operation, but are expected during maintenance events. For example, when a cleaning chemical is added, the pH may change dramatically and if left in corrosion detection mode the measurement of this may provide an incorrect diagnosis corresponding to potential corrosion and a positive corrosion state. In one embodiment, the combination of measurements can be used to detect that it was a planned event and identify this in the diagnosis, ignoring the corrosion threat. In another embodiment, the maintenance mode may be selected wherein the threshold range of the first and further parameter are adjusted to correspond to the expected values of the threshold range during the planned maintenance event. For example, if it is expected that the pressure will increase to a certain level, then the threshold is adjusted correspondingly. However, the new maintenance threshold range will be in place to provide a diagnosis or alert if the pressure changes beyond the new maintenance threshold range. The mode can then be changed back to corrosion detection mode after the planned maintenance event has been completed, and the threshold range will be changed back to the normal operating range. In some examples, the emergency threshold range for each parameter is also correspondingly changed to reflect the expected values, and to identify a problem during a planned maintenance event.

For example, the cumulative dissolved oxygen levels may be monitored during a maintenance sequence. This may be in addition to instantaneous parameters. For example, the cumulative dissolved oxygen levels will provide an indication of the total oxygen in the system over a certain time frame (e.g. one week of pre-commissioning processes). This information can be used by manufacturers of HVAC systems in issuing warranties and determining whether the system has been used within the terms of the warranty.

In some cases the maintenance threshold range will be the same range, but shifted from the normal threshold range. In other cases, the maintenance threshold range will be broadened, for example the lower limit decreased and the upper limit increased from the normal range.

The maintenance mode can be used to prevent the system incorrectly assuming there is a positive corrosion state during a planned maintenance event. This can be done by refining the diagnosis using measurements of different parameters. For example, if the pressure is measured to be too low, the potential consequence could comprise that oxygen may enter the system due to air being drawn in. However, this may occur during a planned maintenance event. To avoid this problem, a different parameter such as the flow rate may also be measured, and if this measurement is within the threshold range, then the system understands that it is a planned maintenance event and not due to air being drawn into the system. In this manner, other parameters can be used to provide information that the system can use to identify a maintenance event, and its success or failure, as well as any problems that occur during the event.

For each maintenance event the threshold range for some or all of the system parameters may be changed accordingly. The comparison of the parameter values to the threshold range allows a diagnosis to be provided which may be sent as an alert. This may comprise that the value is outside the normal threshold or that activity has occurred. In another example, this may comprise that the maintenance event has been successful by monitoring various parameters that are known to be influenced by each specific maintenance event. In a further example, this may comprise that the parameter value is outside the maintenance threshold which was expected, and the system may need attending to, and that the maintenance task may not have been successful as a result.

As an example, during the pre-commissioning process a cleaning chemical may be added after dynamic flushing, the system may be flushed with fresh water, and then a chemical inhibitor may be added. During these three events, various parameters can be monitored to observe the effectiveness of the event and to see if any issues arise. For example, dynamic flushing may be detected by a flow sensor. Dynamic flushing involves removing debris from the system by flushing the system with water (this fresh make-up water may comprise a high dissolved oxygen concentration) at high flow rates. In addition, a cleaning chemical is added to the water. When a cleaning chemical is added to the aerated water supply, the dissolved oxygen concentration and rate of corrosion may increase initially, possibly detected by a change in galvanic current or crevice corrosion rate. This will also be accompanied by a change (often a drop) in pH and a change (usually an increase) in conductivity. By monitoring these parameters, a cleaning maintenance event can be identified and differentiated from a malfunction. Once dynamic flushing has ceased, the dissolved oxygen concentration will eventually drop. Following this, in response to a fresh water flush, the system may experience a rise in dissolved oxygen levels along with a rise in flow rate, a reverse change in pH (i.e. a rise in this case), and a change in conductivity (usually a decrease, since fresh aerated water will have a lower conductivity than that containing cleaning chemicals). For instance, the spike in dissolved oxygen would ordinarily alert the system to a potential corrosion threat. By taking the other parameters into context, the maintenance event can be identified. In response to inhibitor then being added to the system, the dissolved oxygen level may decrease, followed by an increase in conductivity, and a change (e.g. a rise and subsequent plateau) in pH, along with a possible decrease in galvanic current or crevice corrosion rate. In another example, in the case that the system is in maintenance mode and the system is expecting certain maintenance events, this allows the monitoring of the effectiveness of the maintenance event and ensures it was carried out correctly. In some cases, this may be used in conjunction with water sampling. Methods of water sampling, such as following BSRIA guidelines, may provide indications of when certain events have occurred. For example, after a flush has commenced, samples may be taken to determine the amount of iron. The amount of iron may be measured every hour in order to detect a change (usually an increase). The flushing may then continue until the amount of dissolved iron plateaus. The teachings of the present disclosure can be used in addition to these water sampling techniques in order to provide a more accurate and safe overview of maintenance events.

For example, maintenance events may include, but are not limited to: dynamic flushing, addition of cleaning chemical, fresh water flush, addition of chemical inhibitor, heating of the system.

Dynamic flushing may be achieved by an operator attaching a separate hose to the system. The flow rates may then be measured by having the water meter in-line with this part of the system.

Other parameters such as pH and conductivity are often related to the specific chemicals being used and vary depending on the manufacturer. In this case, the expected values of pH and conductivity would depend on the system, and thresholds could be determined based on datasheets. In any case, a user may ensure that the dissolved oxygen concentration was not elevated for too long (e.g. by taking an integral under the graph of dissolved oxygen against time).

Optionally, the processor is configured to send a human-readable message alert in response to the diagnosis. For example, if a diagnosis is provided following a measured parameter being determined to be outside the threshold range (e.g. dissolved oxygen concentration is too high), then a message may be sent that displays this information to the user. The message may comprise an assessment of the cause of the problem (e.g. air has been drawn into the system). The message may also comprise the potential consequences of the problem (e.g. the oxygen may lead to corrosion of metal within the system).

Optionally, the apparatus further comprises a communications unit for outputting the human-readable message alert. In some cases, this may be a transmitter that transmits a wireless signal such as via SMS, or it may comprise an interface with a computer system that allows the message to be sent via email. In other examples it may transmit the message to a display screen that may be positioned locally or remotely, or alternatively may be a virtual screen such as on a web interface, or be connected to a building management system (BMS).

Optionally, the apparatus further comprises a display screen for displaying the human-readable message alert. In some cases, the display screen is connected to the processor via the communications unit. In some examples the display screen is attached to the processor, or may be located adjacent to the apparatus. In other examples, the display screen may be positioned remotely.

In some cases, the message may further comprise a suggestion of how to correct the problem (e.g. attend to pressurisation unit). If the diagnosis has been refined by multiple parameters being measured, then this information can be used to narrow the cause of the problem and provide more precise correction solutions. For example if the dissolved oxygen concentration is increased, this could be due to high pressure or a water leak. By combining the measurement with a measure of the pressure, a possibility can be eliminated and the cause can be identified.

In another embodiment, the message may occur when the system is in maintenance mode and comprises reporting on the performance of a planned maintenance event. For example, this may comprise the successful execution of the event with parameters behaving as expected, or it may comprise an ineffective event describing how a parameter value was outside the maintenance threshold range.

In one embodiment, the message may be displayed on a local screen viewable to a user, or on a web-based dashboard that a user can remotely view for example over the internet. Optionally the user can trigger corrective action by interacting with the alert, e.g. replying to the message or interacting with the dashboard. For example the message may include a button, hyperlink, or other interactive means to allow the user to instruct the system to take corrective action (e.g. shut off a valve, add more inhibitor, heat the water, etc.). Optionally, the message alert could also be sent as an SMS message e.g. to the user's mobile telephone, or as an email sent to specified email addresses. In one example, the message may be available for transmission to a Building Management System. It is appreciated that a skilled person would understand that the message can be displayed in a known manner that is not explicitly disclosed herein.

A user interface such as a dashboard displayed locally or on a web browser can be used to provide updates on measurements and show message alerts. A traffic light colour scheme can be used to indicate when parameters are within acceptable tolerances (green=within threshold range, amber=approaching limit of threshold range, red=exceeding limit of threshold range). Alerts may be automatically triggered when a parameter departs from a threshold range. In other cases, an alarm may be triggered when the cumulative dissolved oxygen or cumulative galvanic current exceeds a certain value.

Optionally, the processor is further configured to take corrective action following the diagnosis. This corrective action may be taken in response to the diagnosis corresponding to the comparison of the results of the measurement of the first parameter with the threshold range, or it may be taken in response to the refined diagnosis corresponding to the further parameter. If the measurement of the first parameter is outside the threshold range and a diagnosis of a positive corrosion state is made, then a corrective action may be taken before the further parameter is used. For example, if the pressure is measured to be too low, then the corrective action may comprise attending to the pressurisation unit and increasing the pressure. This can be helpful, for example, to ensure that any water being drawn in due to the pressure being outside of the threshold range is stopped, thereby allowing the determination of the second parameter value to assess the extent to which the positive corrosion state which has already progressed. In other words, this allows subsequent measurements to be taken while the system is in steady state, thereby meaning the measurements will be valid.

The corrective action may be taken as a result of the diagnosis being presented to the user in the form of a message alert. A corrective action may also occur after a refined diagnosis is provided. The effectiveness of the corrective action can be determined by the measurement of parameters before and after the corrective action is taken. For example, the dissolved oxygen concentration and the galvanic current can be measured before and after a corrosion inhibitor is added to the system, determining if the inhibitor has been effective in suppressing corrosion.

The corrective action necessary may change based on the refined diagnosis. The refined diagnosis may provide an improved suggestion of the cause of the positive corrosion state, and therefore may provide an improved suggestion of how to correct the problem. For example, if the first parameter is dissolved oxygen which is measured to be higher than the threshold range, and the further parameter is pressure measured to be below the threshold range, then the required action is to attend to the pressurisation unit to increase the pressure to within the threshold range.

Optionally, the processor is further configured to automatically perform the corrective action. For example, this may be performed by a computer-controlled system. For example, if the diagnosis comprises the pressure being too high, then the system may automatically adjust the pressure to within the threshold range. In some examples, the user may have to approve automatic corrective action, but after approval the corrective action is still performed automatically by the system. The automatic corrective action may not be the exclusive corrective action, and the user may still be alerted, and it may act as an immediate and temporary solution, for example until the user is able to arrive on site.

Optionally, the processor is configured to perform an automatic corrective action in the event that the value of any determined parameter is outside a corresponding emergency threshold range. For example, an automatic correction could be triggered if the measured parameter values exceed an upper or lower limit of the emergency threshold range. In other examples, the automatic corrective action is performed based on measurements of additional parameters other than the first or further parameters. For example, if the conductivity drops below the threshold lower limit Y, a diagnosis of a positive corrosion state is provided, but if the conductivity drops even further to below the emergency threshold lower limit Z, then an emergency state is activated comprising automatic corrective action being taken. For example, this may comprise the automatic addition of inhibitor in order to improve passivation within the system by bringing the concentration up to the desired range. For example this could be monitored by measuring the conductivity as inhibitor is added. For example, an indication that inhibitor concentration is increasing may be seen by an increase in the conductivity to above the emergency threshold lower limit Z, and optionally further increasing to within the threshold range, above the threshold lower limit Y. This may comprise sending a text or email to a user or alerting a specialist, or causing an alarm to sound in the building. These alerts may be the same as alerts generated if the parameter value departs from the threshold range, or it comprise a different alert if the parameter value departs form the emergency threshold range. For example, if the conductivity decreases below the threshold lower limit Y, then an alert message is displayed on a web interface or building management system, but if the conductivity decreases below the emergency threshold limit Z, then a text message alert is also sent to the user or specialist for quicker response.

Optionally, the apparatus further comprises means for adjusting a parameter. For example, where a corrective action is taken to adjust a parameter, the apparatus comprises means for adjusting that parameter. Optionally, the means for adjusting a parameter comprises one or more of: control of a pressurisation unit, control of make-up water flow rate, control of automatic air vents and pressure relief valves, means for the addition of corrosion inhibitor, means for the addition of an anti-biofilm agent, a heating and/or cooling unit, and/or pH control. In particular, it is preferable for the system to comprise a means of adjusting levels of chemicals (e.g. inhibitors and biocides) such that automatic control of these levels is provided.

Optionally, the apparatus further comprises data recording means. The data recording means may comprise a data logger or a computer. This may be combined with the processor, or it may exist separately. In some cases the data recording means may be the same as the memory unit within the apparatus. Optionally, the data recording means is configured to record the value of any determined parameter continuously or periodically over time. In some embodiments, the parameters can be discretely measured, while in some they can be continuously measured. For example, the pressure may be measured continuously (subject to sampling restrictions) over time, such that a spike in pressure can be recorded. The measurement of the parameters may be recorded on data recording means such as a data logger or computer. In one example, these measurements may be recorded in a table. In other cases, the parameter may be measured at specified time intervals that may dynamically change based on changes in the parameter value. In other examples, the measurement of additional parameters other than the first or further parameters is recorded continuously or periodically over time.

Optionally, the processor is configured to output graphical data based on the recorded parameter values. This may be accessible by the user. For example a graph of the dissolved oxygen concentration may be displayed wherein a change over time may be displayed, even if it remains within the threshold range, allowing preventative action to be taken. The graphical data may be displayed on the screen previously described, or it may be sent via the communications unit previously described to a remote location.

Optionally, the processor is configured to output the graphical data for display in real-time. This allows a user to observe the measurements as they occur, and gain a time-dependent knowledge of how the parameter is changing. Graphical views of historical data can also be provided such that a user can observe how certain parameters changed within a certain interval of time. This information can also be used by manufacturers in assessing the treatment of the system and effective maintenance, which may be used in an assessment of warranty in the case of a broken system. For example, this hard evidence can be used by a company offering a warranty to prove if damage was sufficient to revoke the warranty, for example if a commissioning sequence of maintenance events was ineffectively or improperly carried out. It can also be used by commissioning companies to prove that the maintenance events were carried out correctly, and that they have been effective due to the results of the parameter measurements.

Optionally, the processor is further configured to annotate the graphical data to record planned events or unplanned events, optionally wherein this is performed automatically. The planned events may comprise for example maintenance events such as cleaning events or adding inhibitor, or may comprise leaks or failures of devices such as pressurisation units. This may help the user in understanding the results of the measurements. Unplanned events may be identified as unexpected changes in parameters. For example, it may suggest that a valve has incorrectly been opened. This can be used to inform the user of an equipment failure or operator error. These events can be identified by the monitoring of parameters and comparing the diagnoses to known effects during each planned event e.g. a certain combination of changes to parameters may be indicative of certain events.

Automatic business-logic can be used to interpret measurements and provide an assessment of the event that has occurred based on the changing parameter value. This assessment can occur independently, or can be made in conjunction with a comparison to a threshold value.

This can be used to indicate when a heating or chilled water system has, or is close to, being damaged during commissioning activities or on-going operation. By using message alert indicators on a display dashboard it is possible to see when an HVAC system is being abused due to adverse commissioning or maintenance practices and for example make an informed decision on whether or not the warranty should be invoked. In particular, the extent of cumulative corrosion can be measured by integrating the measured galvanic current with time. In other examples, the cumulative dissolved oxygen levels may be determined by integrating the measured dissolved oxygen at the time of measurement, or at a later stage. This can be especially useful in assessing damage to the system. Alternatively, an algorithm can be used to determine the combined effect of, for example, high dissolved oxygen, low inhibitor levels and high temperatures. The indicator would inform the commissioning or maintenance company when the warranty was near to being transgressed such that appropriate action could be taken. Also, of course, the company offering the warranty would have hard data to prove when the damage was sufficient to revoke the warranty. For example, a manufacturer may use the data of cumulative dissolved oxygen to provide evidence that the total dissolved oxygen in the system over a defined period has exceeded the recommended maximum limit, and therefore the warranty may be void.

Disclosed herein is a method of monitoring a plurality of parameters for detecting corrosion in a closed water system, the method comprising: receiving, from a first sensor, a value of a first parameter selected from the plurality of parameters; comparing the received value of the first parameter to a threshold range for the first parameter; providing a diagnosis of a corrosion state based at least on the comparison of the received value of the first parameter to the threshold range for the first parameter; receiving, from a further sensor, a value of a further parameter, the further parameter being one of the plurality of parameters; comparing the received value of the further parameter to a threshold range for the further parameter; refining the diagnosis of the corrosion state based on the comparison of the further parameter to the corresponding threshold range; wherein each parameter of the plurality of parameters is based on at least one of the following: pressure; make-up water flow rate; dissolved oxygen; cumulative dissolved oxygen; inhibitor dosing levels; biofilm accumulation; temperature; conductivity; galvanic current; cumulative galvanic current; crevice corrosion rate; and/or pH.

In this method the parameters used as the first and further parameters are selected from the plurality of parameters: pressure; flow rate; dissolved oxygen; cumulative dissolved oxygen; inhibitor dosing levels; biofilm accumulation; temperature; conductivity; galvanic current; cumulative galvanic current; crevice corrosion rate; and/or pH. For example the first parameter may be pressure, and the second parameter may be dissolved oxygen. In another example, if the first parameter is dissolved oxygen, and the diagnosis comprises that the dissolved oxygen is above the upper limit of the threshold range, then galvanic current may be used as the second parameter, which can be used to detect signs of corrosion and confirm the positive corrosion state. This can be used to intelligently assess the system health and efficiently and effectively monitor signs of corrosion.

Optionally, the threshold ranges for the first and further parameter corresponds to a normal operating level of the corresponding parameters. If the parameter is within the threshold range, then the system behaves as normal. However, if the value of the parameter is outside the threshold range, this indicates the parameter is outside of the allowable and normal operating range.

Optionally, the diagnosis indicates a positive corrosion state in the event that the value of the first parameter is outside the threshold range for the first parameter. Alternatively, the diagnosis indicates normal system health in the event that the value of the first parameter is within the threshold range for the first parameter. For example if the first parameter value (such as dissolved oxygen) is outside the threshold range, this may lead to corrosion (due to oxygen in the water). In this case, the diagnosis indicates a positive corrosion state which refers to a high level of dissolved oxygen within the water, which may cause corrosion-related problems.

Optionally, in the event of a positive corrosion state, the diagnosis comprises an assessment of the potential causes of the positive corrosion state. The diagnosis may provide an assessment of the most likely causes of the positive corrosion state. For example, if the dissolved oxygen levels are detected to be higher than the threshold range, then this may be caused by several factors e.g. pressure being too low and causing air to be drawn into the system. Alternatively, there may be a leak in the system causing aerated make-up water to be drawn in. The diagnosis can provide the possible causes of the positive corrosion state. This can be used by the user in attempting to isolate the cause of the problem, and in turn rectifying it.

Optionally, in the event of a positive corrosion state, the diagnosis comprises an assessment of the threat to the system health as a consequence of the positive corrosion state. This may include that the cause of the problem may affect the corrosion state in some way. For example, if left unattended, a low pressure reading may lead to corrosion. In other cases, if the galvanic current is too high, then corrosion is already occurring. The level of the threat to system health depends on the parameter being determined.

Optionally, in the event of a positive corrosion state, the diagnosis comprises a suggested correction to rectify the positive corrosion state. This correction may be based on the possible cause of the positive corrosion state. For example, if it is determined that the pressure is too low then the suggested correction may include a correction to the pressurisation unit involving increasing the pressure back into the threshold range.

In some examples, in the event that the value of the first parameter is outside the threshold range for the first parameter, the refined diagnosis comprises: confirming the diagnosis in the case that the value of the further parameter is outside the corresponding threshold range; or correcting the diagnosis in the case that the value of the further parameter is within the corresponding threshold range. In this manner, the diagnosis may be refined with the results of the determination of the further parameter. In one embodiment where the pressure was measured as the first parameter and determined to be lower than the threshold range, a corresponding diagnosis was consequently provided. The diagnosis may for example comprise as assessment of the potential consequences of the problem, which in this case may be that air may be drawn into the system, and an increase in dissolved oxygen concentration may be expected. In this case, if the further parameter is dissolved oxygen, and the measurement of the dissolved oxygen is compared to the threshold range for the further parameter (in this case dissolved oxygen), then this would allow the diagnosis to be updated. For instance, if the measured dissolved oxygen levels are greater than the upper limit of the threshold rage, then this information can be used to refine the diagnosis, confirming that the dissolved oxygen levels have increased.

Optionally, the refined diagnosis comprises a refined indication of a positive corrosion state in the event that the value of the first parameter is outside the threshold range for the first parameter, and in the event that the value of the further parameter is within the corresponding threshold range. Optionally, in the event of a positive corrosion state, the refined diagnosis comprises a refined assessment of the potential causes of the positive corrosion state, based on whether the further parameter is within or outside the corresponding threshold range. Optionally, in the event of a positive corrosion state, the refined diagnosis comprises a refined assessment of the threat to the system health as a consequence of the positive corrosion state. The refined diagnosis may further comprise information relating to the positive corrosion state, and in particular how the value of the further parameter relates to the corrosion state. For example, if the further parameter is galvanic current, and the value is determined to be outside the threshold range, then the positive corrosion state may be indicated in the refined diagnosis, which may include that corrosion is occurring. In this case, the refined diagnosis may comprise information that may help narrow down the cause of the positive corrosion state in conjunction with the diagnosis. The refined diagnosis may also provide information about how the parameter is related to corrosion. For example, in this case, the galvanic current indicates that corrosion is already occurring. However, other parameters such as pressure can be used to provide an indication that corrosion may occur if the problem is not attended to. Optionally, in the event of a positive corrosion state, the refined diagnosis comprises a refined suggested correction to rectify the positive corrosion state. This may involve a suggested corrective action such as adjusting the pressure to within the threshold range if the pressure is determined to be outside the threshold range in the refined diagnosis.

Optionally, the method comprises: receiving, from an additional sensor, a value of an additional parameter selected from the plurality of parameters; comparing the received value of the additional parameter to a threshold range for the additional parameter. Optionally, the method further comprises: refining the diagnosis of the corrosion state based on the comparison of the additional parameter to the corresponding threshold range. Optionally, at least one of the additional parameters is different from the first and further parameters. Optionally, at least one of the one or more additional parameters is the same as one of the first or further parameters. An additional parameter can be used to validate previous values of first and/or further parameters, or may be used to provide further information by measuring different parameters from the list of the plurality of parameters.

Optionally, in the event of a positive corrosion state, the refined diagnosis comprises a refined assessment of the potential cause of the positive corrosion state, based on whether the additional parameter is within or outside the corresponding threshold range. Optionally, in the event of a positive corrosion state, the refined diagnosis comprises a refined suggested correction to rectify the positive corrosion state, based on whether the additional parameter is within or outside the corresponding threshold range.

Optionally, the monitoring is performed in a corrosion detection mode or a maintenance mode, and wherein in the event that it is performed in the maintenance mode, the threshold ranges of the first and/or further parameters are adjusted to correspond to the expected values during a maintenance event. The maintenance mode can be used to adjust the threshold ranges for various parameters for expected planned maintenance events. For example, during cleaning events, parameters such as flow rate and dissolved oxygen may deviate substantially from the normal operating conditions, which would ordinarily cause a positive corrosion state to be identified in a diagnosis. However, when the monitoring is performed in maintenance mode, the parameter threshold ranges can be adjusted according to the range of values expected during each planned maintenance event.

Optionally, the first and/or further parameters to be determined are selected based on a planned maintenance event. For example the further parameter can be selected which will help identify if the maintenance event has been carried out successfully. Optionally, in the case that the monitoring is performed in the maintenance mode, the refined diagnosis indicates at least one parameter being outside the corresponding threshold range but inside the corresponding adjusted maintenance threshold range. This allows the system to continue monitoring the corrosion risk by adjusting the threshold range to allow for expected changes in parameter values during planned events.

Optionally, the method comprises sending a human-readable message alert comprising the diagnosis. Optionally, the method further comprises displaying a human-readable message alert comprising the diagnosis. This message may be sent, for example, via SMS or email, or may be displayed on a local display screen, or via a web interface. The method may involve sending the message to a remote location and then displaying the message at a remote display. The message may comprise information relating to the diagnosis, for example the parameter and its relation to the corrosion state, the potential cause of the problem, the consequences of the problem e.g. may lead to corrosion imminently, or a suggestion of how to correct the problem e.g. attend to pressurisation unit.

Optionally, the method further comprises taking corrective action following the diagnosis. For example if the pressure is too low, a corrective action may be taken which may involve increasing the pressure to within the threshold range. In some cases the taking corrective action is performed automatically. Optionally the method further comprises an automatic corrective action being performed in the event that the value of any determined parameter is outside a corresponding emergency threshold range. The corrective action may be performed automatically which may involve adjusting a parameter to within the threshold range. This may be performed immediately, or may require approval of the user.

Optionally, the corrective action comprises adjusting a parameter, involving one of the following: controlling a pressurisation unit, controlling of make-up water flow rate, controlling automatic air vents and pressure relief valves, adding corrosion inhibitor, adding anti-biofilm agent, heating and/or cooling the system water, and/or controlling pH.

Optionally, the value of any determined parameter is recorded continuously or periodically over time. For example a graph of a parameter such as dissolved oxygen concentration may be displayed showing a change over time. If this involves a significant deviation or a trend, it allows preventative action to be taken, which may be more useful than having a single current value of the parameter. Optionally, graphical data is outputted based on the recorded parameter values. Graphical views of historical data can also be provided such that a user can observe how certain parameters changed within a certain interval of time. Optionally, the graphical data is displayed in real-time. Optionally, the graphical data is annotated to record planned events or unplanned events, optionally wherein this is performed automatically. The planned events may comprise for example maintenance events such as cleaning events. This may help the user in understanding the results of the measurements. Unplanned events may correspond to equipment failure or an operator error.

Also disclosed herein is a sensor for in situ monitoring of system health in a closed water system, the sensor comprising: an inlet for receiving water from the closed water system; an outlet for returning water to the closed water system; and a sensing chamber, having: an outer chamber wall, for retaining water in the sensing chamber; a first measurement surface formed from a first metal; a second measurement surface mounted at least partly within the sensing chamber and formed from a second metal, the second metal being different from the first metal; and a current measuring device connected between the first and second measurement surfaces and configured to measure electrical current flowing between the first and second measurement surfaces as a function of time; wherein the sensing chamber is located between the inlet and the outlet and a flow path for water having a cross sectional area of at least 1 cm² exists between the inlet and the outlet via exposed surfaces of the first metal and the second metal in the sensing chamber; and wherein the exposed surface area of each of the first and second metals is at least 5 cm². A preferential flow path for electrical current is thus provided between the two measurement surfaces through the current measuring device. The measurement surfaces themselves are typically supported in a spaced arrangement by use of grommets, O-rings, etc., thereby ensuring that the current due to the generation of charge on the measurement surfaces due to corrosion is preferentially directed through the current measuring device.

By ensuring that the flow path connects the inlet to the outlet via exposed metal surfaces (as used herein, “metal” can mean a pure metal or an alloy), any galvanic corrosion of the anode will result in a measurable build-up of electric charge on the metals. It is a simple matter to measure this using, the current measuring device e.g. an ammeter or other current sensing means (such as a galvanometer) connected between the two surfaces. Connecting a current sensor between the two surfaces means that charge building up on the metals has a conductive path (via the current measuring device) to flow along to equalise the charge imbalance. By allowing charge to flow through the current measuring device in this way, an electric current can be measured, which is indicative of the amount of charge produced (strictly speaking in steady state after connection for a long time, the current is proportional to the rate at which charge is produced) by the galvanic processes in the sensor. In this way, the current can be continuously or periodically measured to give a real time reading of the amount of corrosion occurring in the system.

Additionally, the measurement of the current as a function of time allows changes in current over time to be monitored. This historical information can be stored locally, for example in a memory, or it can be transmitted elsewhere, for example as part of the overall system health indication described in detail above. That the system allows a continuous flow of actual system water through the sensor means that with time, the measurement surfaces experience passivation and/or corrosion in a manner very similar to the exposed metal surfaces of the water system. This makes the measurement far more realistic than traditional sensors.

The choice of the two different metals/alloys from which the two measurement surfaces are made is guided by certain factors. Firstly, the relative position in the galvanic series will determine which surface forms the anode, and which the cathode. It is important, for example, if the sensor is to be an effective measure of the system health of components made from a particular type of steel, then that type of steel should be included as one of the measurement surfaces. The other measurement surface should be selected as higher in the galvanic series (i.e. more noble), so that the particular type of steel functions as the anode and experience corrosion. Secondly, the further apart the two metals are (i.e. the greater the difference in their electrode potential), the more current will be generated (and the quicker a surface will corrode). This can be used to help boost the signal to noise ratio of the output, and improve accuracy. When the signal is boosted in this way, a calibration of the sensor can account for the fact that the actual corrosion experienced by metals in the system which are not part of the sensor will be lower than those in the sensor.

Broadly, the current generated is proportional to the oxygen content of the water. Increasing the inhibitor concentration suppresses the current by passivating surfaces. By careful calibration of the sensor, the galvanic currents in the sensor can be used to determine the rate of corrosion occurring at the anode in the sensor, which is indicative of corrosive effects in the rest of any closed water system to which the sensor is connected, provided that the sensor comprises the same metals as those which are present in the rest of the system, and that the dissimilar metals are electrically coupled to one another. Under these conditions, the galvanic current of the sensor can be representative of the rate of corrosion of the parts of the system where dissimilar metals are electrically connected to one another. For example, no galvanic current is representative of no (or almost no) corrosion, and higher currents represent higher corrosion rates. The metal from which the anode is formed (which is determined by where in the galvanic series the two dissimilar metals are relative to one another) will be the metal which corrodes. Consequently, this method is able to give an indication of the corrosion rate of the anode metal in the system. Since current sensing means are connected between the first and second measurement surface, an external electrical circuit is provided allowing an electrical current (due to electrons liberated from the anodic reaction) to pass through the current sensing means e.g. (ammeter). This external electrical current is equal to the internal electrical current (movement of ions) flowing through the water (electrolyte). In this context, the measured electrical current, as detected by the ammeter, is equivalent to the rate of liberation of electrons from the anode, which is itself equivalent to the corrosion rate.

Another use of the information provided by the sensor is to determine how effective any inhibitor in the water system is at passivating exposed metal surfaces. Since the passivation of surfaces reduces the rate of corrosion, any current generated by exposed metal surfaces is an indication of incomplete passivation of the surfaces in the system. As such, the sensor may further comprise a processor configured to receive a value for the current from the current measuring device and derive an effectiveness of an inhibitor in the water of the closed water system from the value for the current. This allows the system to use the sensor as a measure of system health by providing an indication of the degree to which exposed metal surfaces in the system have been passivated by the inhibitor. Optionally, additional actions may be taken by the processor based on the determination, for example alerting a user to the situation automatically adding inhibitor to the system, or other actions as set out herein. The current can even be monitored while the inhibitor is added (either automatically or manually), and used to determine when sufficient inhibitor has been added to effectively passivate the surfaces. Passivation can take several days to become effective, so the inhibitor may be added slowly or in batches to ensure that too much is not added.

Using the sensor in this way allows a continuous, live readout effectiveness of the inhibitor in the water system at passivating exposed metal surfaces. In traditional monitoring methods, periodic sampling is used to determine the concentration of dissolved ions (from which an inference is made about the effectiveness of the inhibitor) and the inhibitor concentration (which is compared to a recommended concentration, and the amount of inhibitor is adjusted accordingly). As mentioned above, this is simply a snapshot and may not provide a representative account of the ongoing corrosion situation in the system. Moreover, this infrequent sampling may lead to inconsistencies, for example when the measured inhibitor concentration is adequate (according to manufacturer specification), but the dissolved ion concentration is higher than expected, it is not clear whether a user should add further inhibitor or not. Moreover, the temperature of the water in the system has an effect on how good the passivation is at a given inhibitor concentration. Therefore, intermittent measurements of water from systems where the temperature changes provide little in the way of feedback for addressing the problem. By contrast, the present sensor provides a live readout of the effectiveness of the inhibitor by determining directly whether the exposed measurement surfaces have been adequately passivated. Since this is provided in real time, the reading inherently provides a readout of the effectiveness of the inhibitor at the temperature of the water in the system at any given time, even if that temperature changes with time. This reading frees a user from the conflict described above, as the measurement directly relates to corrosion occurring (or lack thereof), and the concentration of inhibitor can be adjusted based directly on the corrosion which is occurring.

The processor can be provided with a memory which has a relationship between the current and the inhibitor effectiveness pre-loaded into it, and the derivation of the effectiveness is made by consulting the sorted relationship. In any case, the simple relationship that zero current correlates with completely passivated surfaces can be used even in an uncalibrated system. A more nuanced readout is provided by a carefully calibrated sensor.

In some examples of the sensor, the first measurement surface is the inner surface of the outer chamber wall. This allows the outer chamber wall to be made, for example, from a pipe corresponding to pipes from which other parts of the HVAC system are constructed (e.g. pipes of the same size and/or made from the same material). This allows the sensor to provide a realistic measure of corrosion in the system in general, by directly monitoring it in a very similar environment to that found in the rest of the HVAC system. In particular, the smooth, curved surface of a pipe is an exact representation of the pipes in the system, which makes the measurement particularly realistic. Alternatively, the two measurement surfaces may be in the form of inserts inside the sensing chamber. Either way, since it will primarily be the anode which corrodes, the outer wall is preferably not made to be the anode, to ensure that the outer wall of the sensing chamber is not corroded in the process of measuring the corrosion. For example, the outer wall may be formed of a suitable plastic material and two dissimilar metal inserts can be at least partly enclosed in the sensing chamber. In the event that one or both of the inserts is damaged after prolonged exposure to the water of the HVAC system, the insert or inserts may be replaceable, thereby prolonging the lifetime of the sensor.

In any case, the thickness of the anode can advantageously be made no thicker than the pipes of the system. This means that the sensor is configured to malfunction (by complete corrosion of the anode) prior to the pipes of the system corroding away. Whether or not exact corrosion thickness measurements are made in respect of the metals of the system (by integrating the current, as described in detail below), a rough guess at the accumulated damage can be derived by noting that approximately the thickness of the anode measurement surface has been lost from pipes made from the same metal as the anode. This can be detected by the signal from the sensor dropping sharply (since less exposed surface area corresponds to a lower current), and can be used to alert a user to an imminent failure and that system pipes should be inspected and/or replaced. For example, if the pipes of the system have a 1 mm wall thickness, the measurement surfaces can be provided at 0.5 mm thickness to provide a safety tolerance. Other combinations are possible, to account for different pipe thicknesses which may be in use in water systems.

It is envisaged that a variety of sensors may be provided to allow a user to monitor corrosion in different systems. Various combinations of anode thickness, metal, etc. can be provided, so that a user may select the most appropriate one for their system. In some examples, the measurement surfaces are removable and replaceable. This allows a user to purchase a kit, comprising measurement surface inserts of different metal types (e.g. aluminium, copper, steel, etc.) each provided at different thicknesses. Upon installation, the user can select the measurement surfaces appropriately for the system they wish to monitor.

The current measuring device may be configured to integrate the current which it senses over time. For example, the sensor may further comprise a processor configured to: receive a time varying value for the current over a period of time; integrate the time varying value for the current over time; and derive a cumulative loss of metal thickness from the integrated time varying current value. This processor may be the same as the processor configured to receive a value of current from the current measuring device, if provided. In other examples, the processor may be a separate processor. This allows a determination of the total amount of corrosion which has occurred in the system. The chemistry of galvanic corrosion can be thought of generally as reduction of a reactive species (e.g. oxygen) in solution at a cathode with a corresponding oxidation of the anode. The anode corrodes in such processes. Since each incidence of oxidation and reduction causes a set amount of corrosion to the anode with a corresponding amount of charge generated, a measurement of the amount of charge is directly proportional to the number of atoms of the anode which corrode. Since total charge is simply the time integral of electric current, a current sensing means which is configured to integrate the current flowing through it is capable of a proxy measurement of the number of atoms corroded (oxidised) from the anode. By factoring in parameters of the sensor, such as the type of metal (or alloy) from which it is made, the exposed surface area of the metal of the first and second measurement surfaces, etc. the amount of corrosion can be normalised to give a thickness of metal corroded. This can be extrapolated to the rest of the system and used to determine whether the system as a whole conforms to relevant safety provisions, or whether repair of replacement of components should be enacted.

Using the sensor in this way allows a continuous, live readout of the rate of corrosion and an indication of the thickness of metal lost in the system. Unlike traditional methods which periodically sample the water in the system and are limited to soluble corrosion products, this sensor can detect metal loss which results in both soluble and insoluble corrosion products. This is because the formation of either soluble or insoluble species will result in a current between the measurements surfaces (which flows through, and is measured by, the current measuring means). Consequently, a more accurate picture of the effects of corrosion on the system is obtainable in this way, compared with traditional methods. By carefully calibrating the system, a realistic, real-time picture of the corrosion state of the system can be provided to a user.

For example, the system can be calibrated (either prior to installation or on site) by weighing a section of pipe or a flat piece of metal (known as a metal coupon) and installing it in the system having water of a given inhibitor concentration, temperature, pH, dissolved oxygen concentration, etc. The current formed in a sensor of the type described above can be measured over time and the result integrated with respect to time. The accumulated current can be recorded and the section of pipe removed from the system. When the pipe section or coupon is cleaned, dried and weighed again, the total amount of lost metal can be calculated easily. This can be converted to a thickness by simple geometry, and the rate of loss (e.g. in mm/year) can be calculated for the current measured. In this way the proportionality constant between the current produced by the sensor (which is itself a function of the two metals making up the measurement surfaces, the exposed surface area, properties of the water such as pH dissolved oxygen, temperature, etc.) and the rate of corrosion of exposed surfaces in the system can be derived. An integration of the current between two times t₁ and t₂ can provide the total thickness of metal lost to corrosion in that time period.

The first and second measurement surfaces are formed from different metals. In particular, the two metals have different nobilities, and have a different position on the galvanic series. The first and second measurement surfaces may be formed of different metals or semi-metals or alloys. In order to improve the correspondence of the sensor output with the health of the system in general, the first and second metals can be selected from the list of metals commonly used to construct closed water systems or their components. In particular, commonly used metals in the construction of closed water systems are steel; copper; lead; aluminium; and alloys thereof. Specific alloys used in such systems include brass, for example. In some cases, for example, the pipes of a HVAC system may be formed from copper, while the valves may be made from brass. Therefore, by manufacturing the sensor with these metals and/or alloys exposed to the water of the HVAC system, the sensor can provide a realistic sample measure of corrosion in the HVAC system, which is representative of the actual corrosion throughout the system. For many systems in practice it will be sufficient to consider only the least noble metal (i.e. the one furthest down the galvanic series) as dominating the overall corrosion. In the above example, brasses tend to be more noble than copper, and so the copper pipes would be corroded in preference to the brass valves.

At least the anode, and in some cases both measurement surfaces are formed from a metal representative of exposed metal surfaces in the closed water system. This improves the measurement because while no current being detected can be related both to no corrosion occurring and to completely effective passivation of the surfaces in the system (or to an absence of dissolved oxygen), when the measured current is non-zero the data can be harder to interpret. Specifically, it is difficult to determine inhibitor effectiveness or a rate of corrosion of metals from a given current when the measurement surfaces are not representative of the metal surfaces in the rest of the system. Since it is predominantly the anode which corrodes, the benefits of choosing the anode to be a metal in the system are clear. However, even choosing the cathode to be a metal representative in the system can be advantageous. As noted above, the rate of corrosion of the anode depends on the material from which the cathode is formed. Therefore, a more realistic picture of the rate of corrosion in the system is provided by ensuring that the cathode of the sensor is representative of the metals in the system too, so that the sensor is a more representative environment, which in turn makes the analysis of the data more straightforward. On the other hand, a large difference in electrode potential between the metals of the two measurement surfaces can boost the signal from the sensor, whether or not the metal of the cathode is present in the system. For example, copper may be advantageously used as the second measurement surface, whether or not it is present in the rest of the closed water system (although often copper is present). Typically, the first or second metal includes iron, copper or aluminium. In some cases, all three metals may be present in the sensor.

In some examples, the inlet and outlet are spaced apart by at least 10 cm, and more preferably by at least 30 cm. Similarly, the sensing chamber preferably has an internal cross-sectional area of at least 2 cm². This measurement refers to the area enclosed by the inner surface of the inner wall of the sensing chamber, even though some of this area may be taken up by one or more measurement surfaces. Moreover, as noted above, the total surface area of the first and/or second metal exposed to the interior of the sensing chamber is at least 5 cm², but in some cases, larger areas may be present. For example, exposed metal surface areas of at least 10 cm², at least 80 cm², or even at least 200 cm² may be present. In sensors of this type, a larger surface area corresponds to a larger current for the same rate of corrosion. Consequently, a larger device (e.g. larger sensing chamber defined by size of outer wall and distance between inlet and outlet) allows for a greater exposed surface area over which water flows (i.e. a larger exposed surface area of metal in the flow path). This in turn increases the current delivered in a given set of corrosion conditions. Since a larger signal is easier to measure and improves the signal to noise ratio of the measurement, a more accurate measurement of corrosion rate is obtained from such a sensor. On the other hand, smaller sensors are generally cheaper to produce, and require less installation space. This can allow multiple sensors to be provided, each configured to track corrosion of a different metal in the system.

While corrosion is an important process in HVAC systems, the actual rate of corrosion can be relatively small, and hard to measure in known sensors. The present disclosure balances the competing constraints of providing a sufficiently large surface area of exposed metal to provide a reasonable signal against that of keeping the overall size of the device small enough that the device is not unwieldy. This sensor does not require a water sample to be taken from a closed water system. Taking a sample in this way typically results in aeration of the sample, which is clearly not representative of the water within the system. The present sensor provides a sensor which does not require water samples being taken out of the system, and allows for in situ measurement of galvanic currents.

To this end, one or both of the measurement surfaces may be formed in a shape to increase its surface area. In the simplest case, it may be that a measurement surface is hollow, thereby approximately doubling the exposed surface area. In other examples, the or each measurement surface may be ridged, corrugated or have the form of a honeycomb or sponge-like structure or other complex folded or high surface area shapes.

Whichever of the above designs is used, the surface area of exposed metal should be known so that the rate at which surface thickness is being lost to corrosion can be accurately determined. This is an important step in calibrating the sensor.

In some examples, the inlet and/or the outlet include a fitting for connecting the sensor to a closed water system. This allows the sensor to be easily installed in an existing system. In some examples, the length of the sensor, or more specifically the distance between the inlet and outlet may be selected to be a standard distance, e.g. a standard pipe length, so that a section of pipe in a HVAC system can replaced with the sensor. The fitting(s) at the inlet and/or the outlet may be suitable for connection to a standard pipe size, for example pipes of outer diameter 15 mm, 22 mm, or 28 mm. In some examples, the inlet and/or outlet may be provided with appropriate screw threads, solder ring fittings, flanges, O-rings, and so forth, depending on the type of system to which the sensor is to be fitted.

In some examples, the second measurement surface may be completely enclosed in the sensing chamber. This can help to ensure that as much galvanic current is generated from the metal of the second measurement surface as possible.

Also disclosed herein is a method of monitoring system health in a closed water system in which inhibitor is used to passivate exposed metal surfaces, the method comprising: providing a first measurement surface of exposed metal in the flow path of the closed water system; providing a second measurement surface of exposed metal in the flow path of the closed water system, where the first and second measurement surfaces are made from different metals and are electrically isolated from one another; measuring a current between the first and second measurement surfaces; and deriving a measure of the effectiveness of the inhibitor from the measured current. This has the advantages set out above in respect of the use of a sensor to determine the effectiveness of inhibitors in the system.

In some cases, in the event that the inhibitor is determined to be ineffective, a user is alerted. Additionally or alternatively inhibitor may be automatically added to the system in response to a finding that the inhibitor is ineffective.

In still more cases, the method further includes deriving an estimate of the inhibitor concentration from the derived measure of effectiveness of the inhibitor. This can be achieved by providing a correlation (e.g. in a memory) between the derived inhibitor effectiveness and the inhibitor concentration. In other cases, the current may be directly related to the inhibitor concentration.

Also disclosed herein is a method of monitoring system health in a closed water system in which inhibitor is used to passivate exposed metal surfaces, the method comprising: providing a first measurement surface of exposed metal in the flow path of the closed water system; providing a second measurement surface of exposed metal in the flow path of the closed water system, where the first and second measurement surfaces are made from different metals and are electrically isolated from one another; measuring a time varying current between the first and second measurement surfaces; integrating the time-varying current with respect to time and deriving a measure of the thickness of metals corroded in the system from the integrated time-varying current. This has the advantages set out above in respect of the use of a sensor to determine the rate of corrosion in the system.

In the methods disclosed above, the current may be measured after the first and/or second measurement surface has been exposed to water in the system for at least one day. This allows the measurement surfaces to “age” in the water. That is, the exposed surfaces of the sensor interact with the water in the system and settle down to a steady state which is representative of the metals in the system. Putting this another way, traditional sensors for measuring currents formed during corrosive processes are typically cleaned between measurements. This is important, as traditional methods take samples from water systems, and may measure a series of samples from many different systems back to back. In order to prevent cross-contamination, it is important to clean such a sensor thoroughly. The information provided by the presently described sensor and sensing method described herein is actually improved by not cleaning the sensor between/during measurement. Ageing the measurement surfaces in the system water in this way makes the measurement more realistic and representative of the actual situation experienced by the metals in the system.

Also disclosed herein is an inhibitor monitoring system for determining the concentration of an inhibitor in heating, ventilation and air conditioning (HVAC) system, comprising: a memory for storing correlations between conductivity values and inhibitor concentrations; a conductivity sensor for determining a value of the conductivity of water in the closed water system; and a processor, wherein the processor is configured to: receive a determined value of the conductivity of water in the closed water system; receive a determined value of temperature of water in the closed water system from a temperature sensor; compare the determined conductivity to a correlation between conductivity values and inhibitor concentrations stored in the memory; and determine an inhibitor concentration based on the comparison of the conductivity values to the correlation, accounting for the effect of the determined temperature in the correlation. Inhibitor concentration levels are notoriously difficult to measure accurately. The present disclosure concerns the finding that a proxy measurement of the inhibitor levels via a conductivity measurement can result in improved manner of the determination of the inhibitor levels over a direct measurement using other, known methods. For example, the present disclosure does not require costly site visits and lengthy analysis off-site, as measurement and analysis is virtually instantaneous. In addition, the measurements do not require aeration of the water which can lead to inaccuracies of the measurements, as well as increased risk of corrosion as a whole. Furthermore, this instantaneous system can be linked to an alarm system to inform the user when a significant deviation occurs (e.g. the inhibitor level is below a certain threshold).

The processor may be further configured to control the conductivity sensor, optionally wherein the processor is configured to cause the conductivity sensor to determine the conductivity and to send the determined conductivity value to the processor. For example, the processor can be configured to trigger the sensor to take a conductivity reading and send the reading to the processor. In this way, the processor can be configured to receive conductivity measurements only when it requires them, for example when a user or other process requests such measurements.

The processor may be configured to receive determinations of conductivity periodically or continuously. This allows for monitoring the inhibitor levels and how they progress with time, for example to add inhibitor as part of a feedback loop and stop adding inhibitor when the desired concentration has been attained.

The system may include a calibration function in which the correlation is provided by performing a series of determinations of conductivity at known inhibitor concentrations. This can ensure that the system is adapted to the specific system which it is intended to measure.

The memory may be populated with the series of determinations of conductivity at known inhibitor levels. This allows the system to ensure that the correlation is available for future measurements. In some cases, older correlation values in the memory are overwritten with subsequent determinations. This ensures that the latest available information is provided.

The processor may be configured to receive a temperature determination from the temperature sensor and wherein the provision of an inhibitor concentration accounts for the effect of the determined temperature in the correlation. The system may further include a heating and/or cooling unit and the effect of temperature is accounted for by holding the temperature at a constant value during the determination of conductivity. Each of these features allows the system to adapt to changing temperature conditions, and to prevent this from affecting the determination of the inhibitor concentration. Optionally, the processor is configured to control the temperature sensor, optionally wherein the processor is configured to cause the temperature sensor to determine the temperature and to send the determined temperature value to the processor.

Alternatively, the processor is configured to account for the effect of temperature by using previously measured data on the correlation between conductivity values and inhibitor concentrations at various temperatures. This can be applied in conjunction with recently measured temperature data, for example from the temperature sensor. Once more this allows the system to provide an inhibitor concentration from measured or determined conductivity data which has been adapted to take temperature effects into account.

In the event that no exact match exists between the temperature of the water in the closed water system and a temperature at which a correlation between conductivity and inhibitor concentration has previously been determined, the processor may be further configured to extrapolate or interpolate between correlations obtained previously at different temperatures. This allows the system to account for the effect of temperature, even when no data exists at that temperature. In some cases, the system may take a simple linear interpolation/extrapolation from known temperatures. In other cases a more complex fitting formula may be used. In some cases, the processor is further configured to store the extrapolated or interpolated correlation in this way in the memory. This allows the memory to gradually fill up with correlations at different temperatures, and thereby speed up the process in the future. Depending on the inhibitor, the correlation may be positive.

The comparison may be performed by reading from memory one or more of: a look-up table; or an equation. That is to say, during calibration, extrapolation or interpolation, the correlation may be fit to a known equation linking the conductivity to the inhibitor concentration by adjusting coefficients in a generalised version of the equation. This can save storage space in the memory where an exact closed form exists for the correlation (or where an approximation in exact form is sufficiently close), since only the equation and the coefficient values need be stored in the memory. In other cases, the calibration may store known inhibitor values against measured conductivity values in a tabular format. Intermediate values between known concentrations can be filled in by interpolation. This method is useful where no appropriate closed form exists for describing the correlation.

As an example, different inhibitor types may be used, which each have a different form of correlation. The relationship between conductivity and concentration is different for different soluble chemicals as it depends on electrical charge and ionic mobility, for example. The relationship for a given inhibitor may be predetermined before operation of a system. Along with the inhibitor relationship, the base water profile can also be taken into account. For example, hard water has a higher conductivity than soft water. As such, in some cases the base water information is stored and used when determining the relationship. The exact choice of inhibitor may be made based primarily on external factors so it may be that the selected inhibitor is not known when the system is installed, or that the type of inhibitor is changed part way through the life cycle of the system. Consequently, the inhibitor monitoring system may include a plurality of correlations stored in the memory, wherein each correlation corresponds to a different inhibitor, and wherein the processor is configured to use the correlation corresponding to the inhibitor currently being used in the closed water system. In some cases, some of the correlations may be stored as equations, and some as tabular data, depending on the most appropriate form for that inhibitor. In any case, the system can be updated with any changes in inhibitor type, to ensure that the correct correlation is used in determining the inhibitor concentration.

In any case, the inhibitor may be selected from many commercial inhibitors available. For example, common inhibitors are nitrate/nitrite mixtures as well as molybdates, chromates or tungstate, although others will be known by the skilled person. In some cases, the inhibitor used may be a mixture of chemicals, of which the composition is not entirely known by the user. Other chemicals may be used such as ones that buffer pH to slightly alkaline. The present disclosure allows the use of any inhibitor, provided the relationship is pre-programmed. Typically, the relationship is positive for most commercial inhibitors. However, glycols used to prevent freezing in chilled water systems may reduce the conductivity rather than increase it (as other inhibitors do). As such, a different relationship may be calculated when glycols are also being used in the system.

Where multiple inhibitor correlations are stored in the memory, these most commonly used ones may be stored in the memory. For example the correlations may be provided pre-loaded into the memory such that no calibration is required prior to use.

Also described herein is an inhibitor monitoring method for determining the concentration of an inhibitor in heating, ventilation and air conditioning (HVAC) system, comprising: storing correlations between conductivity values and inhibitor concentrations; determining a value of the conductivity of water in the closed water system; determining a value of temperature of water in the closed water system; comparing the determined conductivity to a correlation between conductivity values and inhibitor concentrations stored in the memory; and determining an inhibitor concentration based on the comparison of the conductivity values to the correlation, accounting for the effect of the determined temperature in the correlation.

Optionally, determinations of conductivity are made periodically or continuously.

In some examples the correlation is provided by performing a series of determinations of conductivity at known inhibitor concentrations. Optionally, the series of determinations of conductivity at known inhibitor levels populate a memory and/or wherein older correlation values in the memory are overwritten with subsequent determinations.

The accounting for the effect of the sensed temperature in the provision of an inhibitor concentration is achieved in some cases by holding the temperature at a constant value using a heating and/or cooling unit during the determination of conductivity.

In other cases, the effect of temperature is accounted for by using previously measured data on the correlation between conductivity values and inhibitor concentrations at various temperatures. In some cases, the output of the conductivity sensor is compensated for temperature. Therefore, for a constant dosing level, the output of the conductivity sensor is not affected by temperature. Moreover, in some examples extrapolating or interpolating between correlations obtained previously at different temperatures is performed, in the event that no exact match exists between the temperature of the water in the closed water system and a temperature at which a correlation between conductivity and inhibitor concentration has previously been determined. These correlations may be stored for future use.

The correlation may be positive and/or monotonic.

In some cases, the comparison is performed by using one or more of: a look-up table; or an equation.

Optionally a correlation is selected from a plurality of correlations, wherein each correlation corresponds to a different inhibitor, and wherein the method includes using the correlation corresponding to the inhibitor currently being used in the closed water system.

The similarities to the inhibitor monitoring system described above will be apparent to the skilled person, as will the correspondence between the advantages of the method and the stated advantages of the system.

Optionally, the apparatus for monitoring a plurality of parameters for detecting corrosion in a closed water system previously described further comprises the sensor described above. The advantages of the sensor can be applied to the apparatus disclosed by one of the additional sensors being the sensor. Therefore the technical effects of the sensor described above can be achieved through incorporation into the apparatus for detecting corrosion. In this manner, the processor is configured to: refine the diagnosis of the corrosion state based on the comparison of the galvanic current to a threshold range for galvanic current stored in memory. In this manner, the apparatus can use the determined value of the galvanic current to provide or refine the diagnosis and improve the knowledge of the corrosion state.

Optionally, the apparatus for monitoring a plurality of parameters for detecting corrosion in a closed water system previously described further comprises the inhibitor monitoring system described above. Optionally, the apparatus for monitoring a plurality of parameters for detecting corrosion in a closed water system previously described further comprises the sensor described above and also further comprises the inhibitor monitoring system described above. The advantages resulting from the technical effects of the inhibitor monitoring system can be applied to the apparatus disclosed. In some cases, the processor and memory of the inhibitor monitoring system may exist separately to the processor and memory of the apparatus. In other cases, the functionality may be combined, and the resources may be shared in the combined system. For example a single processor may perform the processing tasks of the combined system, and/or the same memory unit may be used in the combined system. This resource sharing can improve efficiency and provide holistic control by integrating separated features.

Optionally, the method of monitoring a plurality of parameters for detecting corrosion in a closed water system previously described further comprises the steps of the inhibitor monitoring method described above. By incorporating the steps of the inhibitor monitoring method, the combined method gains the technical advantages of the inhibitor monitoring method.

In some cases, the inhibitor concentration measurement may be supplemented with the current measurements described above to determine how effective the inhibitor is at that concentration. As noted briefly above, the inhibitor concentration is only half the story. Differences between the test system in which the optimal inhibitor concentration is derived by the inhibitor manufacturer and the real world system in which the inhibitor is being used can result in the optimal concentration being different from the recommended value. A combination of the two measurements allows a user to add inhibitor and monitor in real time how effective the inhibitor actually is at passivating exposed surfaces in the system. The concentration can thereby be adapted to ensure that surfaces are adequately passivated (according to system or user requirements), without wasting excess inhibitor. Overdosing does not usually lead to negative effects on system health (and it is therefore safer to overdose where a user is not certain), but has cost and environmental implications.

Disclosed herein is an optical sensing apparatus for mounting in a water system and for monitoring corrosion in the water system, comprising: a metal sample having a uniform thickness and a first planar surface and a second planar surface opposite the first planar surface, wherein the first planar surface is arranged to be in contact with water within the water system; a light source configured to emit light towards the second planar surface of the metal sample; and a light sensor configured to receive light reflected by the second planar surface of the metal sample, and output a signal indicative of the intensity of the reflected light.

Since the metal sample is of uniform thickness, the appearance of pinhole corrosion on that sample provides a warning that pinholes of that depth are present in the system. It can be hard to predict where on a sample pinhole corrosion will occur, as it is driven by localised variations such as minor imperfections of the metal on a small scale or gaps in passive layers. Consequently, as well as providing a realistic representation of e.g. pipes in a system (which have a uniform thickness), providing an area of metal of uniform thickness, with one side exposed to the system water, means that wherever on the first planar surface the pitting starts, it will have the same thickness of metal to travel through before it becomes visible on the second planar surface, thereby causing a change in the light reflected from the second surface. The detection of a change in reflectivity in this way provides an indication that pinholing has occurred and that the depth of pitting in the system is at least as deep as the thickness of the metal sample. Indeed, the time at which pinholing is first detected indicates the time at which the pitting reaches that depth. This in turn allows monitoring of the system health in that a comparison may be made against a maximum safe pit depth for the system, which may take into account factors such as: temperature of the water in the system; pressure of water in the system; materials from which the system is constructed (e.g. to assess strength); thickness of components of the system (e.g. pipe wall thickness); and so forth. Should the safety threshold be exceeded, then part or all of the pipes or other components of the system can be flagged for replacement, repair, or any other appropriate remedial action. It should be noted that the terminology “pinhole” usually refers to a hole formed entirely through a wall, while “pit” usually refers to a hole which extends part way through a wall.

Putting this another way, as corrosion progresses, the film will gradually pin-hole changing the visual appearance of the inner surface. This visual change manifests itself as an increase in the number and/or size of dark circles as corrosion debris forms and grows from the pin-hole edge. This change in appearance can be detected by changes in reflectivity of the light emitted from the light source and reflected from the metal sample, as detected by the light sensor as described above.

An estimate of the rate of pitting can be obtained by dividing the thickness of the metal sample by the amount of time which has elapsed since the sample was installed in the system water. This estimate can be used to provide an advanced warning of when the system is expected to reach an unsafe level of pinhole corrosion, so that maintenance can be scheduled in advance of the system requiring such maintenance, thereby improving efficiency. Indeed, in some cases attention can be directed to the causes of corrosion such as high dissolved oxygen or unfavourable pH.

Sensors for detecting uniform corrosion exist which have a wedge-shaped sample. As corrosion progresses, a uniform amount of metal is removed from a surface of the metal sample exposed to system water. This uniform corrosion is monitored by such a sensor because a uniform thickness of lost metal results in complete loss of metal at the thin end of the wedge. As uniform corrosion progresses, more and more of the wedge is completely dissolved. There is therefore a linear relationship between uniform thickness lost across the wedge and amount of light reflected by the metal because where metal is lost, light is not reflected by the wedge-shaped sample. While such systems seem to provide a convenient method of measuring uniform corrosion, they are entirely unsuited to monitoring pinhole corrosion. It is also expected that some corrosion information might be lost due to the physical arrangements of the light source and detector. This is because the non-uniform thickness of a wedge-shaped sample will show pinhole corrosion at the thinner end first. Unless efforts are made to correlate the existence of a pinhole with the location of the pinhole and the original thickness of the wedge at that location, then no meaningful information on the depth of pitting in the system can be gleaned. Typically such systems are incapable of determining the location of pinholes with sufficient accuracy to correlate this with a thickness of metal, and therefore to provide a useful indication of the severity of pitting and pinholing in the system.

Pinholing shows up primarily in two ways. First, the pinhole extends through the metal, causing a small hole of missing metal. Reflection occurs from the metal of the second surface, so where a pinhole appears this manifests itself in the absence of reflection in that area. However, as noted, pinholes tend to be relatively small in diameter (leading to a small area of metal missing for reflection), and therefore this effect is not large. The second evidence for pinholing is tarnishing of metal around the pinhole. This occurs because system water can penetrate through the metal to contact parts of the second surface. This contact causes parts of the second surface to corrode, causing discolouration and further metal loss. In addition, the side walls of the cylindrical pinhole undergo standard uniform corrosion, which causes discolouration of the area of the second planar surface around the pinhole, even where direct contact between system water and the second planar surface is not possible. After time, the uniform corrosion of the side walls of the cylindrical pinhole causes widening of the pinhole and a larger area of missing metal, thereby enhancing the first effect described above.

It will be appreciated that the present invention is also suitable for detecting uniform corrosion inasmuch as where pinholing does not dominate, the complete loss of a sample can be an indication that a uniform thickness of metal (i.e. the thickness of the sample) has been lost from the system.

As used herein, the term “metal” includes elemental metals such as iron, copper and aluminium, as well as alloys such as brass and stainless steel. In order to improve the signal to noise ratio further, the reflectivity of the second surface of the metal sample can be provided with an increased reflectivity, for example by polishing that surface. This allows as much incident light as possible to reflect from the second surface, and thereby maximising the difference between parts of the sample which are reflecting the light and those which are not, whether due to tarnishing or outright absence. A large difference such as this results in a larger change in received light, and consequently makes even small changes easier to detect. To ensure accuracy and capturing of all corrosion effects on the thin film surface, the inner walls of the housing (i.e. an optical cavity, where the light source and the light sensor are housed), is coated by a highly diffuse reflective paint. This gives light emitted by the light source more interaction with the surface of the metal sample. It also gives the system the ability to work with different metal sample surface finishes.

In some examples, only the first planar surface of the metal sample is arranged to be in contact with the water within the water system. That is, the second planar surface of the metal sample is arranged to not be in contact with the water within the water system. The second planar surface may be prevented from contacting the water within the water system. This is to ensure that corrosion on the second surface is only able to occur, and be optically detected, when corrosion (e.g. pinhole corrosion) corrodes from the first planar surface through the metal sample. Therefore, when pinholes are present on the second planar surface and detected by the light sensor, it can be determined that pinhole corrosion has occurred through the metal sample from the first planar surface in contact with the water, through to the second planar surface. To achieve this, water is prevented, restricted, or otherwise inhibited from contacting the second planar surface. For example, the apparatus may be configured such that the only way to contact the second planar surface is to corrode through the metal sample from the first planar surface. That is, the only way that system water can contact the second planar surface is to pinhole or otherwise corrode through the metal sample from the first planar surface.

Optionally, the optical sensing apparatus further comprises a transparent element disposed at least partly between the light source and the metal sample. For example, this may be formed from glass or transparent plastic or the like. The fact that the element is transparent means that it does not block the transmission of light to or from the second planar surface, thereby ensuring that the operation of the device is not impeded. In addition, the metal samples of the system may be selected to have a specific thickness in order that pinholing of that thickness indicates pitting has occurred to that depth. This thickness is selected independently of the required strength of components which contact the water of the water system (e.g. independently of any requirement to withstand a particular system pressure). Therefore, the transparent element can help to provide strength to the metal sample. The transparent element can be selected to be a suitable thickness to withstand the pressure of any given system. “Transparent” in this context means that the element blocks little or none of the light emitted by the light source. That is to say, the element may be chosen so that its absorption and/or reflection spectrum is relatively low at wavelengths of light emitted by the sensor and/or reflected from the second planar surface.

Optionally the transparent element has a third planar surface arranged adjacent to the second planar surface of the metal sample. This transparent element protects the second planar surface from exposure to the water of the system, thereby ensuring that the type of corrosion which results in a detectable change in the reflected intensity is primarily that due to pinholing. The third planar surface can be arranged in contact with the second planar surface, or otherwise in such a way as to form a seal to prevent the second planar surface being in contact with system water. The transparent element may have the same shape and size as the metal sample to assist in this.

Optionally, the optical sensing apparatus further comprises a seal for protecting the second planar surface from water in the water system. As noted above, this seal may be formed in part by a transparent element. Alternatively this may include O-rings or other suitable sealing means. Sealing the second planar surface from the system water can help to ensure that the corrosion must corrode through the entire thickness of the sample before it becomes detectable, thereby preferentially focussing the measurement on pinhole corrosion. In addition, since the light source and light sensor are arranged to respectively shine light at and receive reflected light from the second planar surface, many arrangements position these components on the same side of the metal sample as the second planar surface. In other words, the metal sample may be located between the system water and the light emission and sensing parts of the apparatus. Arrangements which seal the second planar surface against the system water can also have the effect that the light emission and detection means are also protected from system water. This can help to protect e.g. electronic components from damage by exposure to system water. In some cases, both the metal sample and the transparent element may have a seal to prevent system water from flowing past them. The metal sample may be sealed to protect the second planar surface from system water (at least until a pinhole corrodes entirely through the metal sample). The transparent element may be sealed so that even when pinholes have corroded holes through the entire thickness of the metal sample, the light source and sensor are protected from system water and its associated damage. In some cases, both seals may be provided by a single seal.

Optionally the seal may be located adjacent to the first planar surface. This allows a single seal to be used to protect the light source, light sensor and second planar surface. Further optionally, the metal sample may be provided with a corrosion-resistant coating on the first planar surface in the vicinity of the seal. It has been found that corrosion is concentrated in locations near to a seal due to what is known as crevice corrosion. This distorts the amount of corrosion detected by the system by artificially creating a preferential site for corrosion. It has been found that a thin layer of corrosion-resistant coating between the metal sample and the seal prevents preferential crevice corrosion in the vicinity of the seal. The corrosion-resistant coating should be selected for its durability. Since the device is anticipated to leave a sample in contact with system water for many years, the corrosion-resistant coating should: withstand temperatures encountered in water systems (typically 5° C. to 100° C.); resist corrosion from the water, dissolved oxygen, inhibitors, sterilisation products (e.g. chlorine), impurities (dissolved metal ions, salt, etc.); survive in such environments for a long period of time (e.g. 5 to 10 years); prevent corrosion of the underlying metal sample; be compatible with the underlying metal sample; and be applicable to the underlying metal sample without causing oxidation or otherwise reacting with the sample, in order to not alter the chemical properties of the metal sample at the edges of the corrosion-resistant layer, which can distort the corrosion.

The metal sample may be representative of one or more metals used in the water system. For example, the pipework may be made from carbon steel, stainless steel, copper, etc. Pumps and valves, etc. may be made from stainless steel, aluminium or brass, while solders formed from alloys of many metals (tin, copper, bismuth, indium, zinc, silver, manganese, antimony, lead, cadmium, aluminium, etc.) may be used to form joints in the system. By choosing the sample to reflect the metals or alloys used in the system, a realistic picture of the actual corrosion happening to that metal in the environment of the system can be obtained. It is important to emphasise that the corrosion which occurs to the metal sample is directly representative of the corrosion which occurs to elements in the water system formed from the same metal as the metal sample because they are both exposed to the same water (the system water).

The uniform thickness of the metal sample may be 1 mm or less, for example from 0.025 mm to 1 mm, or from 0.05 mm to 0.5 mm. Since pipe wall thicknesses in systems tend to be 1 mm or more, the use of metal samples of 1 mm or less can provide advanced warning of the failure of such systems, so that remedial action can be taken in advance of an expensive failure.

Optionally, the apparatus may include a plurality of metal samples. This can allow corrosion of different types of metal to be monitored by providing samples of different metals. In other cases, a series of samples of different thicknesses may be provided. This can provide a more compact sensor than providing a separate sensor for each thickness of metal and/or each different metal type. Of course, an alternative arrangement is to provide a separate sensor for each metal and/or thickness. Where a single sensor has a plurality of metal samples, the sensor may share various measurement components. In some cases, the plurality of metal samples includes N metal samples and wherein the sensing apparatus comprising fewer than N light sources and/or fewer than N light sensors. The sensor may use multiplexing to correlate the received light with the plurality of metal samples. For example the multiplexing may include one or more of: time division multiplexing; wavelength or frequency division multiplexing; special division multiplexing; and/or polarisation division multiplexing.

For example, a single light source (or in general fewer light sources than there are samples) can be arranged to shine onto the second surface of multiple metal samples, thereby ensuring that each sample is illuminated with the same light, so allowing a meaningful comparison of the received light intensity, as the light received by the second surface of each sample can be controlled to be the same in each case. In other cases, there may be a single light sensor and multiple light sources. For example, each light source may emit light in a narrow wavelength range and the sensor may be configured to detect the received intensity broken down by received wavelength, thereby allowing the sensor to correlate intensity in a wavelength with a particular sample (known as wavelength division multiplexing), and thus monitor several samples using only one sensor, or more generally using fewer sensors than there are samples. An alternative method of sharing light sources and light sensors is to use a directed light source and/or light sensor and illuminate the second planar surface of different samples at different times (known as time division multiplexing), and synchronise the sensor with the light source, so that light received at a given time is correlated with a given metal sample.

An alternative method of sharing light sources and light sensors is to use a directed light source and/or a light sensor having a narrow angular range of sensitivity and illuminate the second planar surface of different samples at different times (known as time division multiplexing), and synchronise the sensor with the light source, so that light received at a given time is correlated with a given metal sample. The light source and/or light sensor may be arranged to direct or receive (respectively) light in narrow angular ranges, and to change the regions to which the light is directed or received over time. For example, by physically rotating a light source, or manipulating mirrors, lenses, etc. to direct the emitted light to a different location. This can be used to shine light at different metals samples at different times, thereby providing spatial multiplexing. In some examples, polarised light may be used to multiplex the signals (polarisation-division multiplexing). As an example, a light source may be split into two orthogonal polarisation directions and each different polarisation directed towards a different metal sample. Light sensors can be fitted with polarisers to ensure that only a particular polarisation is detected by the sensor. Not only does this reduce the number of light sources required, but it can help to reduce cross-talk as light polarised in an orthogonal direction to a given sensor's polariser cannot enter the sensor.

In yet another case, other aspects such as processors for interpreting the received intensity data or housings for enclosing the sensors (both of which are described in more detail below) may be shared between multiple metal samples. In general, it will be seen that multiplexing in time, wavelength, etc. can be used to monitor multiple samples while sharing some of the monitoring equipment such as light sources, light sensors and/or processors. While wavelength division multiplexing can be more expensive to provide, it can provide more subtlety as the required sensors can help to determine type of corrosion as well as simply detecting some form of corrosion. A cheaper alternative is to match the light source with the light detector based on prior experimental data and use the matched pair for each surface. On the other hand, costs and complexity may be kept down by using an appropriate time division multiplexing. Is some cases, it may be helpful to combine different types of multiplexing to achieve the desired effect.

In some cases the plurality of metal samples each have a different thickness, as set out below, in order to determine the maximum pitting corrosion depth. The samples may be provided as a series of samples of thicknesses, for example, 0.025 mm, 0.05 mm, 0.075 mm, 0.1 mm, 0.125 mm and so forth. As set out below, this allows the maximum pitting depth to be estimated to the nearest 0.025 mm.

In some examples the (or each) metal sample is replaceable. This may be achieved by use of access hatches or the like for accessing the metal sample, removing the sample, and replacing the metal sample. In other cases, the apparatus may be mounted on a bypassable section of pipe, e.g. with another portion of pipe running in parallel to the section of pipe having the sensor and with valves for directing flow along either section of pipe. In normal use the system water is directed to flow past the portion of pipe to which the apparatus is mounted. When the sample in the sensor is to be replaced, the flow can be diverted along the bypass pipe (the section parallel to the portion with the sensor), and optionally the section of pipe having the sensor apparatus can be drained of system water. It is now possible to remove the sensor from the system without causing a leak. Once the apparatus has been removed, the metal sample can be removed from the sensor and replaced with a fresh one, a different thickness, one formed from a different metal, etc. This arrangement can also be used in the initial installation of the apparatus, and for removing the apparatus itself for cleaning, maintenance, etc.

The apparatus may further comprise a processor configured to receive the signal indicative of the intensity of the reflected light from the light sensor. This can be used to determine the level of corrosion in the water system. In some cases the processor is configured to relate the signal indicative of the intensity of the reflected light to corrosion of the metal sample. For example the processor may be configured to determine that a decrease in the intensity of the reflected light corresponds to an increase in the corrosion of the metal sample, for the reasons set out above. The processor may be remote, in the sense that the signals are fed to e.g. a centralised monitoring station, where signals from multiple sensors are collected and analysed together or individually as part of a holistic health check of the water system. In any case, the reflected intensity from the (or each) sample can be provided as an analogue output, or it can be converted to a universally compatible format such as Modbus for easy transport and centralised processing, for example.

The light sensor may be configured to output a plurality of signals indicative of the intensity of the reflected light over time and wherein the processor is configured to determine a rate of corrosion of the metal sample from the plurality of signals indicative of the intensity of the reflected light over time received from the light sensor. In other words, by monitoring the reflected light signal over time, it is possible to detect how quickly the corrosion is progressing, and thereby provide e.g. updated estimates of the time until the system reaches an unsafe level of corrosion or to predict the remaining lifetime of particular components of the system, so allowing advanced action to be taken. In other cases, the sensor may be configured to issue (and the processor configured to receive) signals at predetermined (e.g. periodic) times. This can allow an ongoing assessment of whether corrosion is occurring and how severe it is at each reading.

Where there are multiple samples present, the processor may be configured to receive a plurality of signals indicative of an amount or a rate of corrosion of a corresponding plurality of metal samples. Thus a series of signals, each corresponding to a particular metal and/or thickness, may be received and the processor can be configured to determine which samples have corrosion (and how much). As above, each set of samples of the same metal can be used to determine a maximum pitting depth for that metal.

The plurality of metal samples may be formed from the same metal as one another, wherein each of the plurality of metal samples has a different thickness and wherein the processor is configured to determine a range of maximum pitting corrosion depths in the water system from the plurality of received signals. In other words, the system may be configured to “digitise” the outputs to determine that the pitting corrosion depth in the system is at least as deep as the thinnest sample which shows pinholing, but is not as deep as the thickest sample which does not show pinholing, with the exact depth between these limits being unknown. By selecting the various samples to be relatively close in thickness, the accuracy of the determination of the depth of pitting in the metals of the system can be improved.

Where multiple samples of different thickness are present, the expected situation is that all samples up to and including a certain thickness show pinholing, while all samples above that certain thickness do not. In some cases however, especially those with samples of very closely spaced thicknesses, it may be that there are gaps in the samples which show pinholing, for example the thinnest three samples may show pinholing, the next (fourth) sample does not, the fifth sample does show pinholing, and all thicker samples do not. This can result from the random nature of pinhole corrosion, for example. Nonetheless, the thickest sample which shows corrosion should be used as an estimate for how far through the metal the corrosion has penetrated. This is primarily for safety reasons, as this sample shows definitive evidence that pitting corrosion has penetrated to this depth in metals of the system.

Of course, it is possible to have a series of thicknesses of samples for each representative metal in the system in some cases. For each series of thicknesses of the same metal, the above considerations apply.

The apparatus may further comprise an optical element configured to direct light emitted by the light source towards the second planar surface of the metal sample, and/or configured to direct the reflected light towards the light sensor. This can be in the form of a lens, a fibre optic cable, a mirror, a prism, etc. In cases where there is a transparent element on the second planar surface of the metal sample, the transparent element can function as the optical element. The purpose of the optical element is to ensure that the light emitted by the light source is directed to the second planar surface, and provides a uniform coverage of light to the second planar surface. This in turn ensures that where it will result in the same change in reflected light intensity, irrespective of where the corrosion occurs on the second planar surface.

The light source and/or the light sensor may be mounted in a housing forming a closed opaque cavity. This can inhibit ambient light from affecting the sensor and providing false positive results. In such cases, a portion of the opaque walls is the second planar surface for receiving and reflecting light emitted from the light source. That is to say, the metal sample forms an opaque portion of the wall. In some of these cases, as described above, the metal sample may be behind a transparent element, meaning that while the metal sample is not technically an internal wall of the housing, it nevertheless provides an opaque surface to prevent light entering the housing.

Internal walls of the housing may have diffuse highly reflective inner surfaces for providing an optical integrating cavity. Optical integrating cavities are chosen so that all light emitted by the light source eventually reaches the light sensor. They have the effect of smoothing out the light intensity in the interior of the housing so that each portion receives approximately the same light intensity. This prevents inhomogeneities in the angular distribution of light emitted from the light source from causing different parts of the sample to be illuminated with different intensities of light and ensures that the background light levels are highly consistent. Such a situation could lead to different parts of the sample showing different effects when corrosion occurs, if the inhomogeneities are severe enough. Of course, providing a highly homogeneous light source is another solution to this. Where the internal walls of the cavity are said to be diffuse reflective surfaces, this does not necessarily apply to the second planar surface of the metal sample, and certainly does not apply to the transparent element, where such an element is present.

The optical sensing apparatus may further comprise a baffle for blocking direct light transmission between the light source and the light sensor. The baffle can prevent the sensor from outputting a false negative, in the sense that if a significant amount of light is transmitted directly from the source to the sensor without reflecting from the metal sample, then corrosion of the metal sample will have less of an overall effect on the received intensity as a portion of the received intensity will always come directly from the light source, irrespective of corrosion of the sample. A baffle can be as simple as an opaque protrusion on an internal surface of a housing positioned between the light source and the light sensor. In other cases, the light source and/or the light sensor may be mounted in a respective recess in order to prevent direct light transmission between the source and the sensor. In such examples, the recess will be arranged so as to provide a path for light from the source to shine onto the second planar surface and/or to provide a path for light reflected from the second surface to arrive at the sensor.

The optical sensing apparatus may further comprise a second light sensor for directly sampling the light emitted from the light source to provide a reference value. By directly sampling light emitted by the light source, the light received by the first light sensor (that is the light reflected form the second planar surface) can be normalised to the intensity (and indeed spectral composition) of the emitted light. This technique provides an effective light intensity referencing scheme to avoid light intensity variation due to drift and temperature changes. As noted above, it is anticipated that the sensing apparatus remains installed in a water system for many years. In some cases, the light source will remain on for this entire time, in other cases, the light source will be switched on and off many times as part of a periodic measurement schedule. In either scenario, the light source is liable to change its output intensity and/or spectral composition over time. By comparing the received intensity at the first detector (after reflecting from the second planar surface) to the received intensity at the second detector (sampled directly from the light source), changes relative to the actual light output can be detected, thereby accounting for the effect of changes in light output over time.

The second sensor may receive light directly from the light source in a variety of ways. For example, the light source may be arranged to emit light into a fibre-optic bundle. Some fibres in the bundle may then be split out of the bundle some way along the length of the bundle and fed to the second sensor, while the remainder of the bundle directs the light towards the second planar surface. In other cases, a mirror may be used in much the same way, by reflecting a part of the light emitted from the light source, while the unreflected part of the beam is unaffected by the mirror and proceeds instead to the metal sample as described above. A related idea is to use a beam splitter (part-silvered mirror, pair of prisms joined to form a cube, etc.) to split the emitted light into two portions, one of which is directed towards the metal sample, and the other towards the second (i.e. reference) sensor.

In some examples the light emitted by the light source has a spectrum selected based on the metal from which the metal sample is made and/or the expected corrosion mode of the metal from which the metal sample is made. For example, since different metals have different colours (that is reflect/absorb different wavelengths of light), the light source may be configured to emit light in a particular wavelength range. For example, a copper sample reflects strongly in the red and orange part of the spectrum (around 600 to 700 nm), but much less strongly in the green, blue and violet parts of the spectrum (around 400 to 550 nm). This means that the contrast between a light source reflecting perfectly from a copper surface and not reflecting (due to tarnishing of the metal, or indeed complete corrosion of the metal) is far less pronounced when the light is in the blue/green part of the spectrum than when it is in the red part of the spectrum. The efficiency of the device can therefore be improved by emitting light only in the regions of the spectrum which produce a high contrast effect. This can include tailoring the emitted light to the wavelength-dependent reflection of the sample, i.e. limiting to red parts of the spectrum for copper, yellow parts for brass, providing uniform wavelength distribution for silvery metals such as stainless steel, aluminium, etc.

In any of the examples presented herein, light sources can be any suitable source, such as incandescent bulbs, halogen bulbs, fluorescent bulbs, LEDs, lasers and the like. Where emission at specific wavelengths or ranges of wavelengths is required, white light sources can be filtered, or inherently limited wavelength sources such as LEDs or lasers can be selected as appropriate. Indeed, while the above discussion makes use of readily understood terms in the context of visible electromagnetic radiation for clarity and ease of understanding, it will be appreciated that the sensor may operate outside of the visible part of the electromagnetic spectrum, for example where specific wavelength regions provide a strong contrast between clean and tarnished metal, for a given metal. In some cases, for example, infra-red or ultra-violet light may be used in the measurement (using a suitable light source and light sensor pairing). In other cases, regions of the electromagnetic spectrum yet further from the visible parts may be used.

In any of the examples presented herein, light sensors can be any suitable sensor, such as photodiodes, phototransistors, photoresistors, CCD devices and the like. Where the light emitted from the light source is limited to a particular wavelength of range of wavelengths, or indeed, the metal sample preferentially reflects particular wavelengths of light, the sensor should be configured to be able to detect these wavelengths, and preferably is particularly sensitive to such wavelengths. In some cases, as set out below, it may be advantageous to filter the particular wavelengths out of light entering the sensor, or to configure the sensor to be particularly insensitive to particular wavelengths. In yet more cases, it may be preferable for the sensor to be able to detect colours (e.g. using an RGB or more complex CCD arrangement) to provide a more nuanced view of corrosion in the metal sample.

In some cases, the spectral composition of the light emitted from the light source may be tailored to the type of corrosion which the metal sample is expected to undergo. This may be in addition to or instead of tailoring the spectrum to the metal itself, as described above. For example taking the copper example above, copper is known to form a green-blue patina under some conditions, where the copper(II) (Cu²⁺) ion forms and binds to chemical species (acetate, carbonate, chloride, etc.). Where this corrosion route is anticipated (from the chemical and physical environment in the water system) red light may be a particularly suitable choice for emission from the light source. This is because the red light will reflect strongly from the untarnished (reddish-orange) surface. Where the surface corrodes entirely, or tarnishes to blue-green, the red light will be reflected only weakly or indeed not at all. In this example, limiting to light emission in the red parts of the electromagnetic spectrum improves the ability of the system to detect corrosion (and particularly pinhole corrosion) by causing a large drop in reflected light intensity because not only does the (relatively small) pinhole fail to reflect light, but the (usually larger) tarnished region also reflects little or no light. In cases where the metal is silver coloured, such as stainless steel, the uncorroded/untarnished reflection profile may be relatively uniform across visible wavelengths and tailoring the light to the expected corrosion routes of that metal may be the best way to ensure there is a high contrast between uncorroded/untarnished and corroded/tarnished states.

In other examples, the light emitted by the light source may be selected to be in one or more wavelength ranges which reflect strongly from both untarnished and tarnished metal. By providing a sensor which is sensitive to each of these wavelengths (and can distinguish between them), it is possible to determine the relative proportions of the metal which are untarnished, tarnished and completely eroded. For example, where the light received is full strength (optionally normalised to the emitted value as described above) in the “untarnished” part of the spectrum, then no corrosion or tarnishing has occurred. As parts of the second surface become tarnished, the proportion of received light starts to split between untarnished and tarnished (with the end being full strength in the “tarnished” part of the spectrum). Where the entire second surface is either tarnished or untarnished, the sum of [proportion of light in the tarnished part of the spectrum] and [proportion of light in the untarnished part of the spectrum] should add to 100%. Where this sum does not equal 100%, the difference between the measured value and 100% gives an indication of the proportion of the second surface which shows complete corrosion. For example, where the normalised value of the received light in the tarnished part of the spectrum is 32%, and the normalised value of the received light in the untarnished part of the spectrum is 51%, then approximately 51% of the surface is untarnished, approximately 32% is tarnished and approximately 100%−(32%+51%)=17% is completely corroded. In some cases, rather than selecting the light source to output two or different wavelengths or ranges of wavelengths, the metal sample may be illuminated with white light, and the separation into reflected wavelength components may be performed by the sensor which can be configured to detect two or more reflected wavelengths or ranges of wavelengths and thereby determine the proportion of the sample which is untarnished, corroded, or tarnished. In some cases, the tarnished category may even be broken down into sub-categories, indicating different types of corrosion. The specific type of corrosion may be used, e.g. to alert a user that the water in the system contains unexpected contaminants, and thereby allow a user to take corrective action.

The apparatus may further comprise control electronics for controlling the light source and/or the light sensor. The electronics can be used to periodically trigger emission of light and a corresponding measurement of the light reflected from the second metal surface. Where there is a second sensor, this too can be controlled by the control electronics. The control electronics can also be used to adjust the brightness or spectral composition of the light emitted by the light source, for example to optimise the contrast between untarnished and tarnished metal for the metal sample in the sensor. The control electronics can also be used to control the sending of the received signals or a determination of the extent or type of corrosion in the metal sample(s) to a remote location.

The apparatus may further comprise an electrical power source for providing electrical power to the light source and the light sensor. In some cases, there is a processor, which is also supplied with electrical power by the power source. As noted, the apparatus may be remote from e.g. a centralised monitoring system, which collates the information from many distributed systems. It may be impractical to transmit power to sensors which are located in a remote location, so providing the apparatus with a power supply such as a battery allows the apparatus to function.

It will be appreciated that the optical sensing apparatus disclosed herein may be provided separately, or may be provided in combination with other apparatus disclosed herein. In particular, the optical sensing apparatus may be used in combination with the apparatus for monitoring a plurality of parameters for detecting corrosion in a closed water system. For example, the apparatus for monitoring a plurality of parameters may additionally comprise the optical sensing apparatus as disclosed herein as one of the sensors used by the apparatus in detecting corrosion. It will also be appreciated that the sample for use in an optical sensor for monitoring corrosion disclosed herein may also be used with the apparatus for monitoring a plurality of parameters for monitoring corrosion.

Also disclosed herein is an apparatus for monitoring a plurality of parameters for monitoring and/or determining overall system health in a water system, comprising one or more of the sensors of as disclosed herein. In particular, the apparatus may comprise the galvanic sensor disclosed herein or the optical sensor as disclosed herein. Pitting corrosion and/or pinhole corrosion is a big problem, and the optical sensor described above provides a convenient way of monitoring corrosion and particularly pitting/pinhole corrosion. A plurality of sensors can be used as part of such a system, as described above to provide an estimate of the maximum depth of pitting corrosion in the system. In particular, monitoring a plurality of aspects of system health in situ, using sensors in contact with system water provides a very accurate and reliable assessment of the health of the system.

Also disclosed herein is a method of monitoring corrosion in a water system, the method comprising: mounting a metal sample in a water system, the metal sample having a uniform thickness and including a first planar surface and a second planar surface opposite the first planar surface, wherein the first planar surface is arranged in contact with water of the water system; emitting light towards the second planar surface of the metal sample; receiving light reflected by the second planar surface of the metal sample; generating a signal indicative of the intensity of the reflected light; and correlating the intensity of the reflected light to corrosion of the metal sample.

Since the metal sample is of uniform thickness, the appearance of pinhole corrosion on that sample provides a warning that pinholes of that depth are present in the system. It can be hard to predict where on a sample pinhole corrosion will occur, as it is driven by localised variations such as minor imperfections of the metal on a small scale or gaps in passive layers. Consequently, as well as providing a realistic representation of e.g. pipes in a system (which have a uniform thickness), providing an area of metal of uniform thickness, with one side exposed to the system water, means that wherever on the first planar surface the pitting starts, it will have the same thickness of metal to travel through before it becomes visible on the second planar surface, thereby causing a change in the light reflected from the second surface. The detection of a change in reflectivity in this way provides an indication that pinholing has occurred and that the depth of pitting in the system is at least as deep as the thickness of the metal sample. Indeed, the time at which pinholing is first detected indicates the time at which the pitting reaches that depth. This in turn allows monitoring of the system health in that a comparison may be made against a maximum safe pit depth for the system, which may take into account factors such as: temperature of the water in the system; pressure of water in the system; materials from which the system is constructed (e.g. to assess strength); thickness of components of the system (e.g. pipe wall thickness); and so forth. Should the safety threshold be exceeded, then part or all of the pipes or other components of the system can be flagged for replacement, repair, or any other appropriate remedial action.

An estimate of the rate of pitting can be obtained by dividing the thickness of the metal sample by the amount of time which has elapsed since the sample was installed in the system water. This estimate can be used to provide an advanced warning of when the system is expected to reach an unsafe level of pinhole corrosion, so that maintenance can be scheduled in advance of the system requiring it, thereby improving efficiency. Indeed, in some cases attention can be directed to the causes of corrosion such as high dissolved oxygen or unfavourable pH.

Sensors for detecting uniform corrosion exist which have a wedge-shaped sample. As corrosion progresses, a uniform amount of metal is removed from a surface of the metal sample exposed to system water. This uniform corrosion is monitored by such a sensor because a uniform thickness of lost metal results in complete loss of metal at the thin end of the wedge. As uniform corrosion progresses, more and more of the wedge is completely dissolved. There is therefore a linear relationship between uniform thickness lost across the wedge and amount of light reflected by the metal because where metal is lost, light is not reflected by the wedge-shaped sample. While such systems seem to provide a convenient method of measuring uniform corrosion, they are entirely unsuited to monitoring pinhole corrosion. It is also expected that some corrosion information might be lost due to the physical arrangements of the light source and detector. This is because the non-uniform thickness of a wedge-shaped sample will show pinhole corrosion at the thinner end first. Unless efforts are made to correlate the existence of a pinhole with the location of the pinhole and the original thickness of the wedge at that location, then no meaningful information on the depth of pitting in the system can be gleaned. Typically such systems are incapable of determining the location of pinholes with sufficient accuracy to correlate this with a thickness of metal, and therefore to provide a useful indication of the severity of pitting and pinholing in the system.

Pinholing shows up primarily in two ways. First, the pinhole extends through the metal, causing a small hole of missing metal. Reflection occurs from the metal of the second surface, so where a pinhole appears this manifests itself in the absence of reflection in that area. However, as noted, pinholes tend to be relatively small in diameter (leading to a small area of metal missing for reflection), and therefore this effect is not large. The second evidence for pinholing is tarnishing of metal around the pinhole. This occurs because system water can penetrate through the metal to contact parts of the second surface. This contact causes parts of the second surface to corrode, causing discolouration and further metal loss. In addition, the side walls of the cylindrical pinhole undergo standard uniform corrosion, which causes discolouration of the area of the second planar surface around the pinhole, even where direct contact between system water and the second planar surface is not possible. After time, the uniform corrosion of the side walls of the cylindrical pinhole causes widening of the pinhole and a larger area of missing metal, thereby enhancing the first effect described above.

It will be appreciated that the present invention is also suitable for detecting uniform corrosion inasmuch as where pinholing does not dominate, the complete loss of a sample can be an indication that a uniform thickness of metal (i.e. the thickness of the sample) has been lost from the system.

The method optionally further comprises generating a plurality of signals indicative of the intensity of the reflected light over time, and determining a rate of corrosion from the plurality of intensities of the reflected light over time. In other words, by monitoring the reflected light signal over time, it is possible to detect how quickly the corrosion is progressing, and thereby provide e.g. updated estimates of the time until the system reaches an unsafe level of corrosion or to predict the remaining lifetime of particular components of the system, so allowing advanced action to be taken. In other cases, the sensor may be configured to issue (and the processor configured to receive) signals at predetermined (e.g. periodic) times. This can allow an ongoing assessment of whether corrosion is occurring and how severe it is at each reading.

Optionally, the light is emitted from a light source and/or the reflected light is detected by a light sensor. Moreover, the light source and the light sensor may be mounted in a housing forming a closed opaque cavity. This can inhibit ambient light from affecting the sensor and providing false positive results. In such cases, a portion of the opaque walls is the second planar surface for receiving and reflecting light emitted from the light source. That is to say, the metal sample forms an opaque portion of the wall. In some of these cases, as described above, the metal sample may be behind a transparent element, meaning that while the metal sample is not technically an internal wall of the housing, it nevertheless provides an opaque surface to prevent light entering the housing.

Internal walls of the housing may have diffuse reflective inner surfaces for providing an optical integrating cavity. Optical integrating cavities are chosen so that all light emitted by the light source eventually reaches the light sensor. They have the effect of smoothing out the light intensity in the interior of the housing so that each portion receives approximately the same light intensity. This prevents inhomogeneities in the angular distribution of light emitted from the light source from causing different parts of the sample to be illuminated with different intensities of light and ensures that the background light levels are highly consistent. Such a situation could lead to different parts of the sample showing different effects when corrosion occurs, if the inhomogeneities are severe enough. Of course, providing a highly homogeneous light source is another solution to this. Where the internal walls of the cavity are said to be diffuse reflective surfaces, this does not necessarily apply to the second planar surface of the metal sample, and certainly does not apply to the transparent element, where such an element is present.

In some examples of the method a baffle for blocking direct light transmission between the light source and the light sensor may be provided. The baffle can prevent the sensor from outputting a false negative, in the sense that if a significant amount of light is transmitted directly from the source to the sensor without reflecting from the metal sample, then corrosion of the metal sample will have less of an overall effect on the received intensity as a portion of the received intensity will always come directly from the light source, irrespective of corrosion of the sample. A baffle can be as simple as an opaque protrusion on an internal surface of a housing positioned between the light source and the light sensor. In other cases, the light source and/or the light sensor may be mounted in a respective recess in order to prevent direct light transmission between the source and the sensor. In such examples, the recess will be arranged so as to provide a path for light from the source to shine onto the second planar surface and/or to provide a path for light reflected from the second surface to arrive at the sensor.

The method may be executed on an apparatus further comprising an optical element configured to direct light emitted by the light source towards the second planar surface of the metal sample, and/or configured to direct the reflected light towards the light sensor. This can be in the form of a lens, a fibre optic cable, a mirror, a prism, etc. In cases where there is a transparent element on the second planar surface of the metal sample, the transparent element can function as the optical element. The purpose of the optical element is to ensure that the light emitted by the light source is directed to the second planar surface, and provides a uniform coverage of light to the second planar surface. This in turn ensures that where it will result in the same change in reflected light intensity, irrespective of where the corrosion occurs on the second planar surface.

The method may further comprise a second light sensor for directly sampling the light emitted from the light source to provide a reference value, wherein correlating the intensity of the reflected light to corrosion of the metal sample includes comparing the reference value to the received light reflected from the second planar surface. In other words, the second light sensor is used to normalise the light received at the first light sensor which has been reflected by the second planar surface of the metal sample. By directly sampling light emitted by the light source, the light received by the first light sensor (that is the light reflected form the second planar surface) can be normalised to the intensity (and indeed spectral composition) of the emitted light. This technique provides an effective light intensity referencing scheme to avoid light intensity variation due to drift and temperature changes. As noted above, it is anticipated that the sensing apparatus remains installed in a water system for many years. In some cases, the light source will remain on for this entire time, in other cases, the light source will be switched on and off many times as part of a periodic measurement schedule. In either scenario, the light source is liable to change its output intensity and/or spectral composition over time. By comparing the received intensity at the first detector (after reflecting from the second planar surface) to the received intensity at the second detector (sampled directly from the light source), changes relative to the actual light output can be detected, thereby accounting for the effect of changes in light output over time.

The method may be performed on a plurality of metal samples are mounted in the water system and wherein each metal sample has a uniform thickness and includes a first planar surface and a second planar surface, wherein the first planar surface of each sample is arranged in contact with water of the water system and wherein: light is emitted towards the second planar surface of each metal sample; light is reflected by the second planar surface of each metal sample and received by a sensor; a signal is generated corresponding to each metal sample, the signal being indicative of the intensity of the reflected light from each metal sample; and corrosion of each metal sample is correlated with the intensity of the reflected light.

The plurality of metal samples may be formed from the same metal as one another, wherein each of the plurality of metal samples has a different thickness to the thickness of the other metal samples. This can allow corrosion of different types of metal to be monitored by providing samples of different metals. In other cases, a series of samples of different thicknesses may be provided. This can provide a more compact sensor than providing a separate sensor for each thickness of metal and/or each different metal type. Of course, an alternative arrangement is to provide a separate sensor for each metal and/or thickness. Where a single sensor has a plurality of metal samples, the sensor may share various measurement components. In some cases, the plurality of metal samples includes N metal samples and wherein the sensing apparatus comprising fewer than N light sources and/or fewer than N light sensors. The sensor may use multiplexing to correlate the received light with the plurality of metal samples. For example the multiplexing may include one or more of: time division multiplexing; wavelength or frequency division multiplexing; special division multiplexing; and/or polarisation division multiplexing.

For example, a single light source (or in general fewer light sources than there are samples) can be arranged to shine onto the second surface of multiple metal samples, thereby ensuring that each sample is illuminated with the same light, so allowing a meaningful comparison of the received light intensity, as the light received by the second surface of each sample can be controlled to be the same in each case. In other cases, there may be a single light sensor and multiple light sources. For example, each light source may emit light in a narrow wavelength range and the sensor may be configured to detect the received intensity broken down by received wavelength, thereby allowing the sensor to correlate intensity in a wavelength with a particular sample (wavelength division multiplexing), and thus monitor several samples using only one sensor, or more generally using fewer sensors than there are samples. In yet another case, other aspects such as processors for interpreting the received intensity data or housings for enclosing the sensors (both of which are described in more detail below) may be shared between multiple metal samples. An alternative method of sharing light sources and light sensors is to use a directed light source and/or light sensor and illuminate the second planar surface of different samples at different times (known as time division multiplexing), and synchronise the sensor with the light source, so that light received at a given time is correlated with a given metal sample.

An alternative method of sharing light sources and light sensors is to use a directed light source and/or a light sensor having a narrow angular range of sensitivity and illuminate the second planar surface of different samples at different times (known as time division multiplexing), and synchronise the sensor with the light source, so that light received at a given time is correlated with a given metal sample. The light source and/or light sensor may be arranged to direct or receive (respectively) light in narrow angular ranges, and to change the regions to which the light is directed or received over time. For example, by physically rotating a light source, or manipulating mirrors, lenses, etc. to direct the emitted light to a different location. This can be used to shine light at different metals samples at different times, thereby providing spatial multiplexing. In some examples, polarised light may be used to multiplex the signals (polarisation-division multiplexing). As an example, a light source may be split into two orthogonal polarisation directions and each different polarisation directed towards a different metal sample. Light sensors can be fitted with polarisers to ensure that only a particular polarisation is detected by the sensor. Not only does this reduce the number of light sources required, but it can help to reduce cross-talk as light polarised in an orthogonal direction to a given sensor's polariser cannot enter the sensor.

In yet another case, other aspects such as processors for interpreting the received intensity data or housings for enclosing the sensors (both of which are described in more detail below) may be shared between multiple metal samples. In general, it will be seen that multiplexing in time, wavelength, etc. can be used to monitor multiple samples while sharing some of the monitoring equipment such as light sources, light sensors and/or processors. While wavelength division multiplexing can be more expensive to provide, it can provide more subtlety as the required sensors can help to determine type of corrosion as well as simply detecting some form of corrosion. A cheaper alternative is to match the light source with the light detector based on prior experimental data and use the matched pair for each surface. On the other hand, costs and complexity may be kept down by using an appropriate time division multiplexing. Is some cases, it may be helpful to combine different types of multiplexing to achieve the desired effect. The method may further comprise determining a range of maximum pitting corrosion depths in the water system from the each of the signals indicative of the intensity of the reflected light from each metal sample. In other words, the system may be configured to “digitise” the outputs to determine that the pitting corrosion depth is at least as deep as the thinnest sample which shows pinholing, but is not as deep as the thickest sample which does not show pinholing, with the exact depth being unknown. By selecting the various samples to be relatively close in thickness, the accuracy of the determination of the depth of pitting in the metals of the system can be improved.

Where multiple samples of different thickness are present, the expected situation is that all samples up to and including a certain thickness show pinholing, while all samples above that certain thickness do not. In some cases however, especially those with samples of very closely spaced thicknesses, it may be that there are gaps in the samples which show pinholing, for example the thinnest three samples may show pinholing, the next (fourth) sample does not, the fifth sample does show pinholing, and all thicker samples do not. This can result from the random nature of pinhole corrosion, for example. Nonetheless, the thickest sample which shows corrosion should be used as an estimate for how far through the metal the corrosion has penetrated. This is primarily for safety reasons, as this sample shows definitive evidence that pitting corrosion has penetrated to this depth in metals of the system.

Of course, it is possible to have a series of thicknesses of samples for each representative metal in the system in some cases. For each series of thicknesses of the same metal, the above considerations apply.

The metal sample may be removeable and/or replaceable and the method may include removing and/or replacing the metal sample. This may be achieved by use of access hatches or the like for accessing the metal sample, removing the sample, and replacing the metal sample. In other cases, the apparatus may be mounted on a bypassable section of pipe, e.g. with another portion of pipe running in parallel to the section of pipe having the sensor and with valves for directing flow along either section of pipe. In normal use the system water is directed to flow past the portion of pipe to which the apparatus is mounted. When the sample in the sensor is to be replaced, the flow can be diverted along the bypass pipe (the section parallel to the portion with the sensor), and optionally the section of pipe having the sensor apparatus can be drained of system water. It is now possible to remove the sensor from the system without causing a leak. Once the apparatus has been removed, the metal sample can be removed from the sensor and replaced with a fresh one, a different thickness, one formed from a different metal, etc. This arrangement can also be used in the initial installation of the apparatus, and for removing the apparatus itself for cleaning, maintenance, etc.

The method may further include controlling one or more of: power; intensity; and/or spectral weight of the emitted light.

Also disclosed herein is a sample for use in an optical sensor for monitoring corrosion in a water system, the metal sample comprising a metal element having: a first surface for exposure to the water of the water system; and a second surface opposite the first surface for receiving and reflecting light; wherein a portion of the first surface is provided with a corrosion-resistant coating for providing a location for forming a seal between the sample and the sensor. As noted above, it has been found that a seal formed directly on the surface of a metal sample can give an unrealistic depiction of the amount of corrosion in the system, as system water tends to disproportionately corrode the sample near to the seal, since it forms a crevice and is therefore susceptible to crevice corrosion, which enhances the rate of corrosion in the crevice regions. By placing a corrosion-resistant coating on the sample, the seal can be formed over the corrosion-resistant coating, which helps to mitigate this effect. In other words, the corrosion-resistant coating allows a seal to be formed between the sample and a sensor which does not alter the corrosion environment of the sample adjacent to the seal. Other parts of the first surface (those without a corrosion-resistant coating) are exposed to system water and corrode in a manner which is the same as other metals in the system, so providing an accurate measure of the corrosion of other instances of that metal where such instances occur in the water system.

The metal element may be planar and/or has a uniform thickness. As noted above at length, a planar and/or uniform thickness sample can be particularly suited to detecting and/or monitoring pitting and pinhole corrosion.

The corrosion-resistant coating may be applied to the edges of the metal element. This placement allows the sample to be mounted using a seal at the edges. This in turn allows a single seal to be used to hold the sample in place on the sensor and to seal the sensor from the system water, thereby protecting sensitive electronic components.

The corrosion-resistant coating may extend between 0.5 mm and 5 mm inward from the edge of the first surface. The metal element may have a width of between 10 mm and 50 mm. The corrosion-resistant coating covers no more than 10% of the first surface. These numbers provide a good balance between preventing unrealistic enhancements of corrosion while leaving a sufficiently large area of metal exposed to system water to provide a representative surface for monitoring corrosion.

The metal element may be disc shaped. Optionally the corrosion-resistant coating is annular. These shapes provide a sample which is easy to seal securely to the sensor.

The metal element may be formed from carbon steel, stainless steel, copper, brass, aluminium, or other materials representative of metals in the water system. By forming the sample from representative metals in the system, an accurate picture can be gleaned of how such metals are faring in other parts of the system.

The corrosion-resistant coating may be stable for at least 5 years when submerged in water of the water system. Additionally or alternatively, the corrosion-resistant coating may be stable when submerged in system water of at least 85° C. These parameters reflect the intended use case, where the sample is used in system water for long periods of time.

It will also be appreciated that the method of monitoring corrosion of a metal sample as disclosed herein may be used in combination with the methods of monitoring a plurality of parameters for detecting corrosion in a closed water system. For example, aspects of the methods of monitoring a plurality of parameters for detecting corrosion as disclosed herein may also comprise the method of monitoring corrosion of a metal sample in a water system.

Any method feature as described herein may also be provided as an apparatus, and vice versa. As used herein, means plus function features may be expressed alternatively in terms of their corresponding structure.

Any feature in one aspect of the invention may be applied to other aspects of the invention, in any appropriate combination. In particular, method aspects may be applied to apparatus aspects, and vice versa. Furthermore, any, some and/or all features in one aspect can be applied to any, some and/or all features in any other aspect, in any appropriate combination. It should also be appreciated that particular combinations of the various features described and defined in any aspects of the invention can be implemented and/or supplied and/or used independently.

A selection of specific examples will now be described in detail to illustrate some of the effects of the system and method described herein, with reference to the Figures, in which:

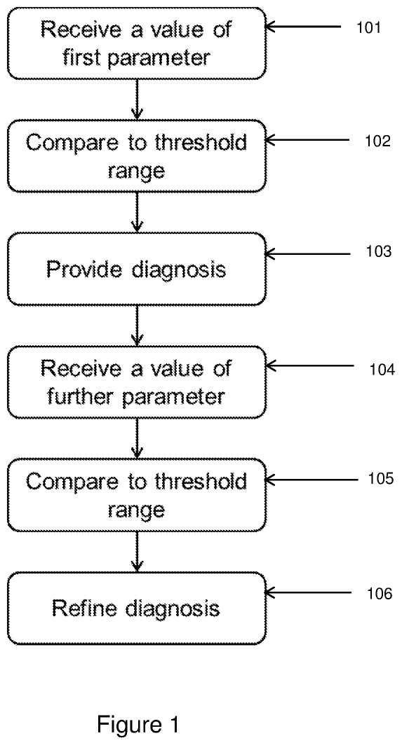

FIG. 1 shows a flow chart representing the method disclosed;

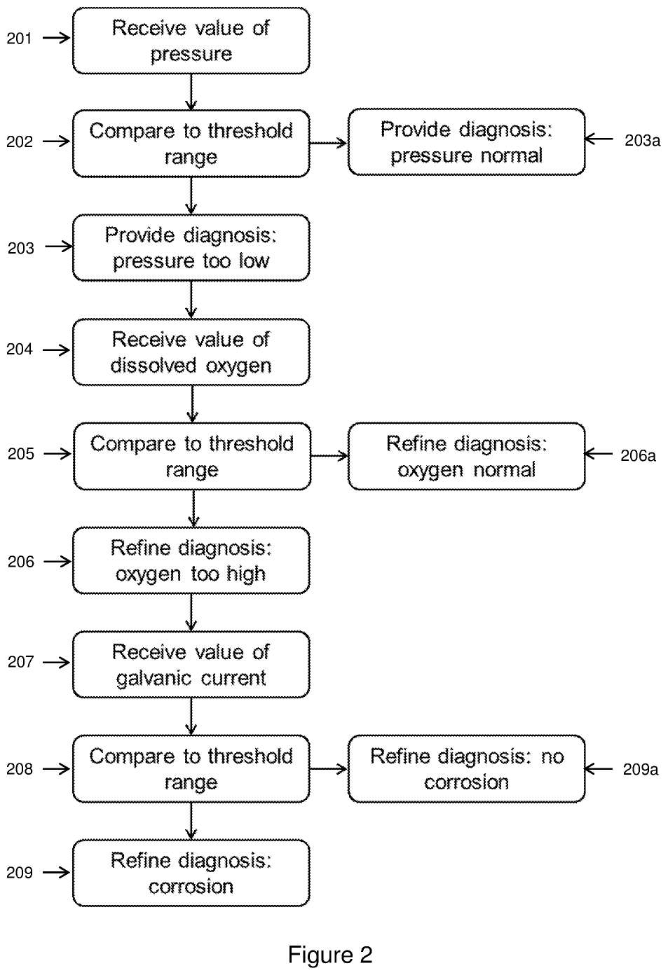

FIG. 2 shows a flow chart representing an example of the method refining a corrosion diagnosis caused by low pressure, according to one embodiment;

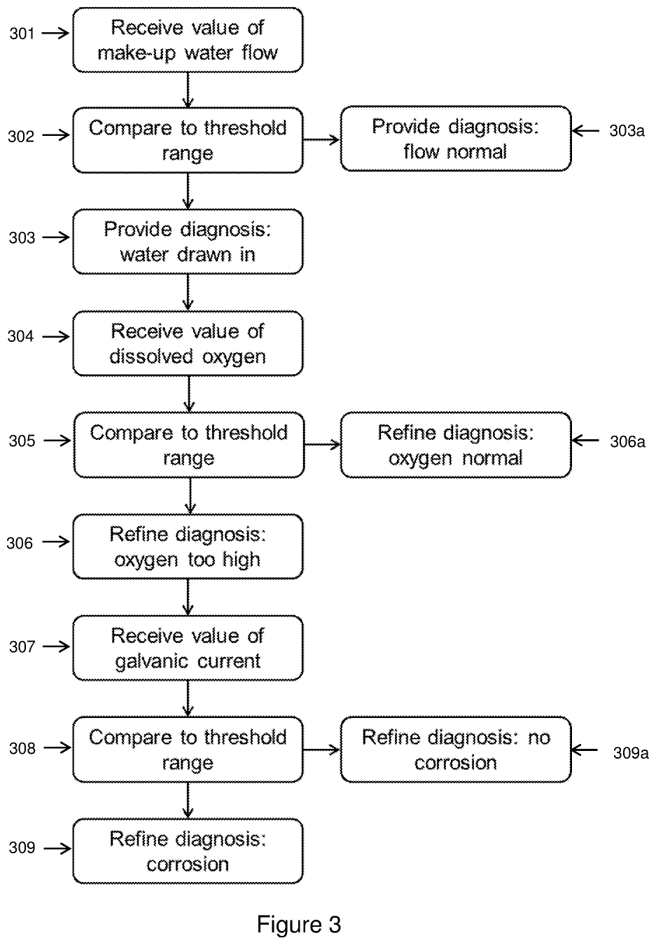

FIG. 3 shows a flow chart representing an example of the method refining a corrosion diagnosis caused by a leak, according to one embodiment;

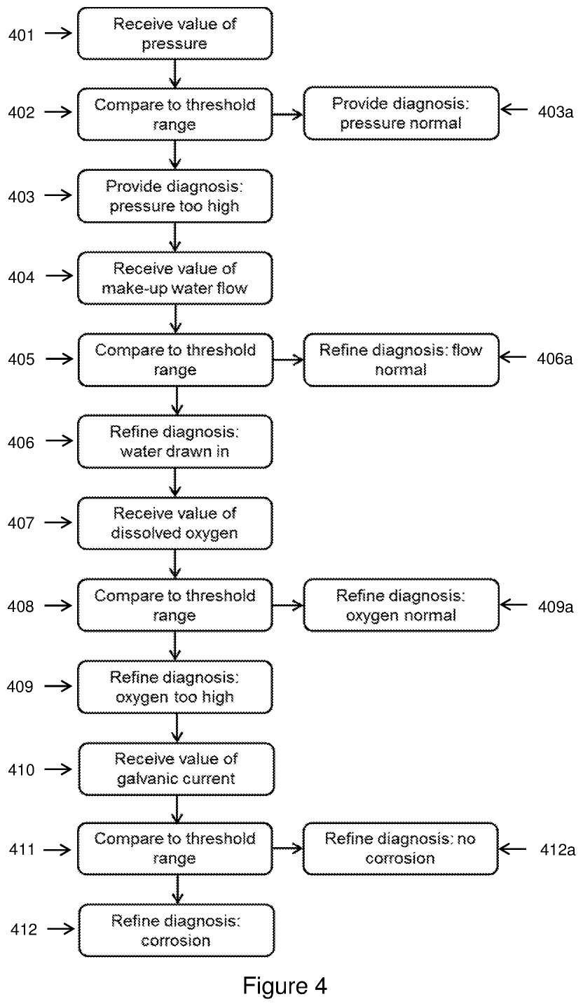

FIG. 4 shows a flow chart representing an example of the method refining a corrosion diagnosis caused by high pressure, according to one embodiment;

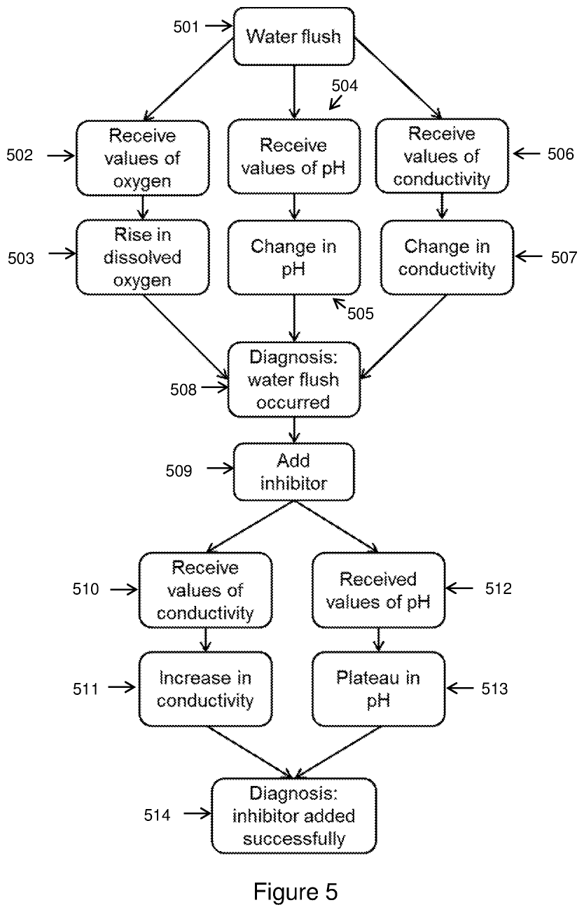

FIG. 5 shows a flow chart representing an example of the method verifying the occurrence of planned maintenance events, according to one embodiment;

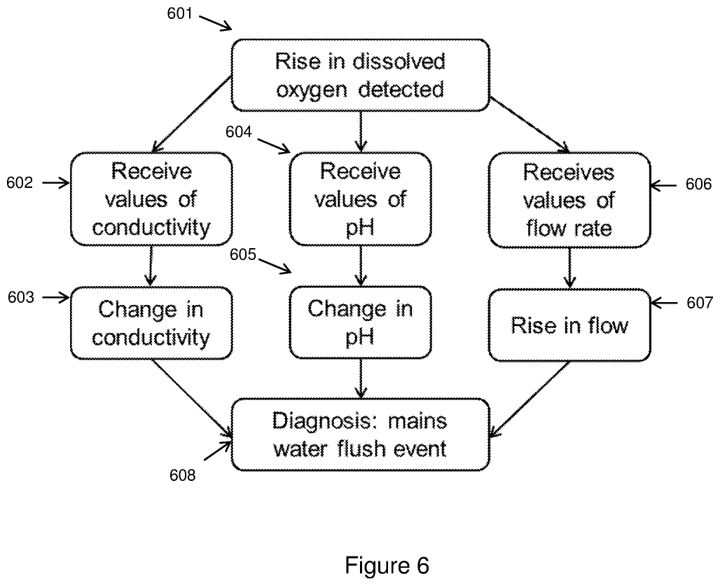

FIG. 6 shows a flow chart representing an example of the method identifying a maintenance event, according to one embodiment;

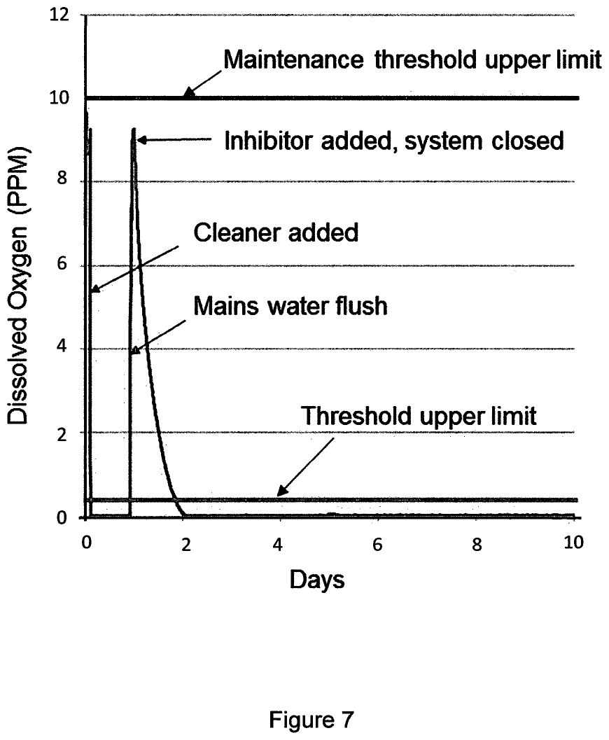

FIG. 7 shows a graph representing changes in dissolved oxygen levels over time due to maintenance events, according to one embodiment;

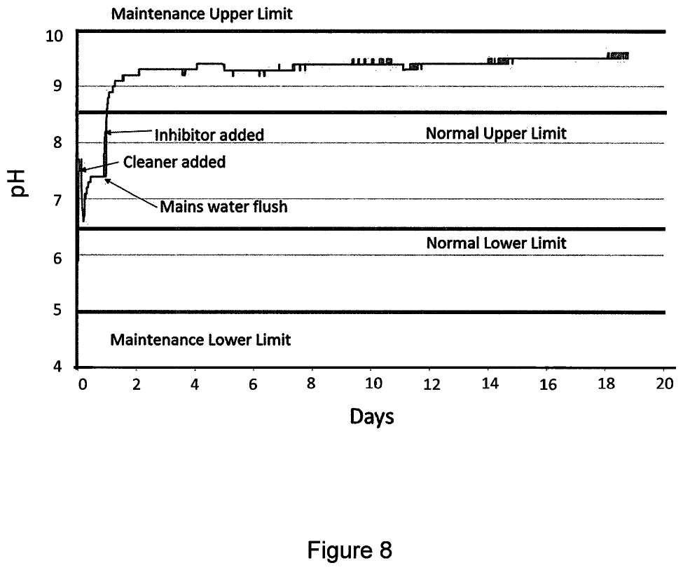

FIG. 8 shows a graph representing changes in pH levels over time due to maintenance events, according to one embodiment;

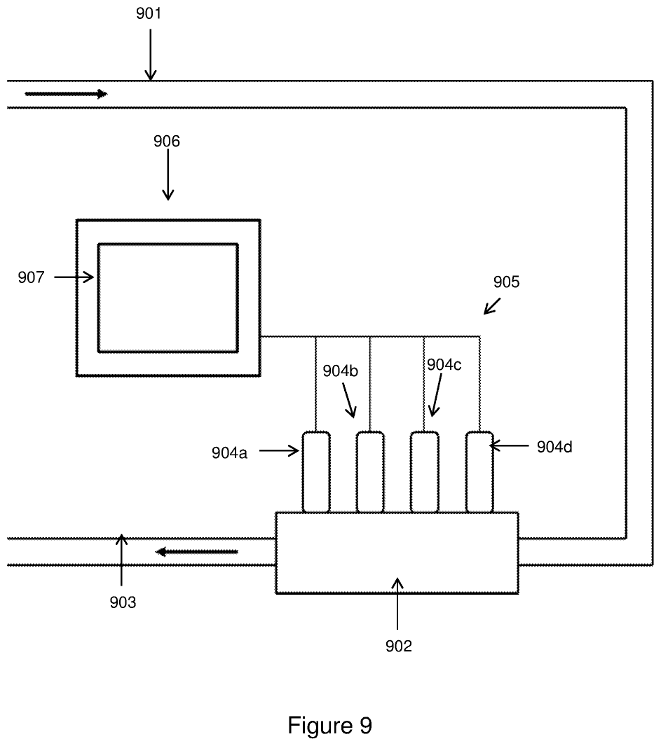

FIG. 9 shows a drawing of a system configured to carry out the method disclosed, according to one embodiment;

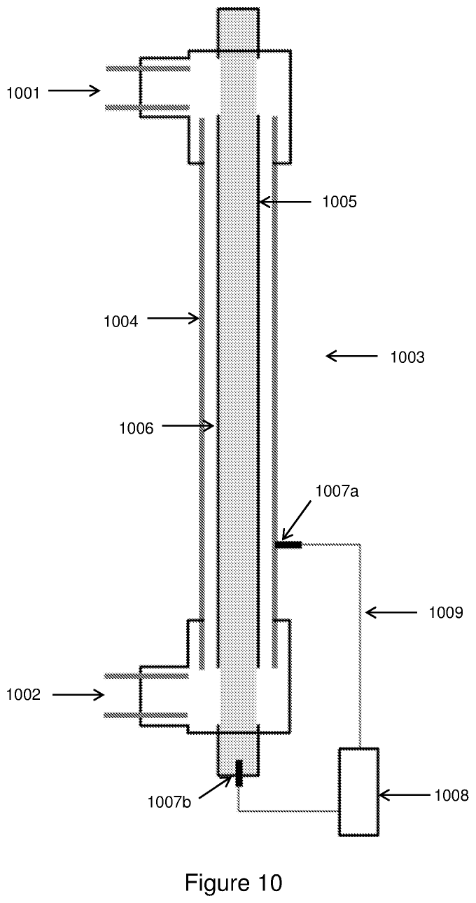

FIG. 10 shows a schematic of a sensor as described herein;

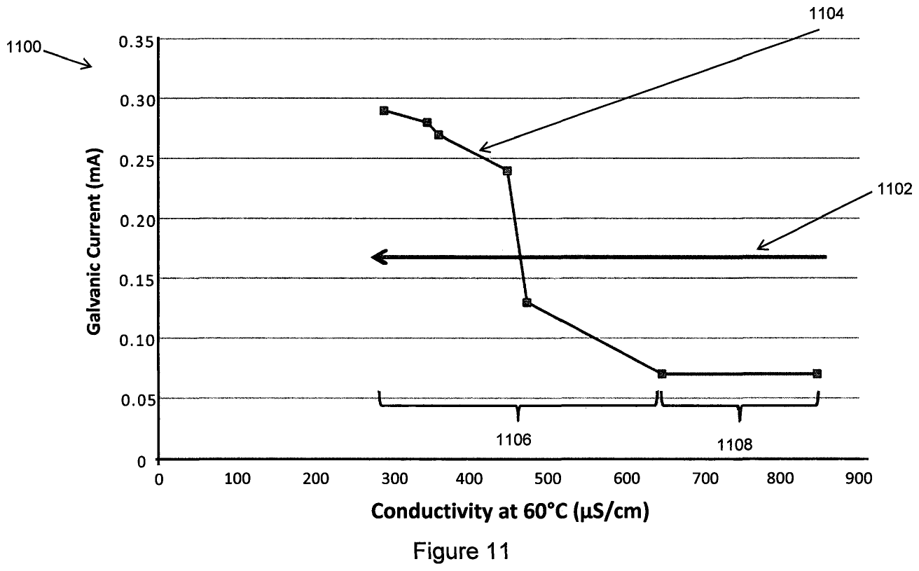

FIG. 11 illustrates the relationship between conductivity, dissolved oxygen and galvanic current;

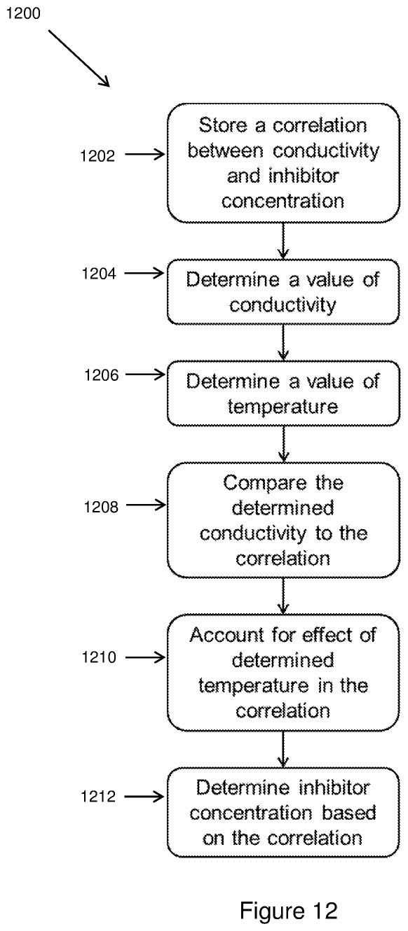

FIG. 12 shows a flow chart of a method of determining inhibitor concentrations from the conductivity of the water, compensating for temperature effects;

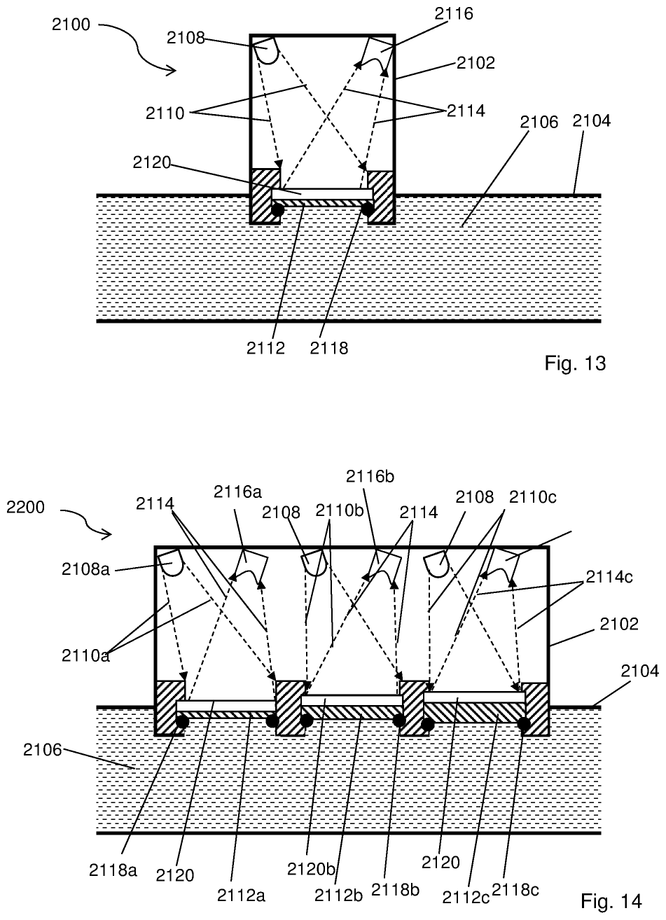

FIG. 13 shows a schematic of an example embodiment of an optical sensor for detecting and/or monitoring corrosion in a water system;

FIG. 14 shows a schematic of another example embodiment of an optical sensor for detecting and/or monitoring corrosion in a water system, having a plurality of metal samples of different thicknesses;

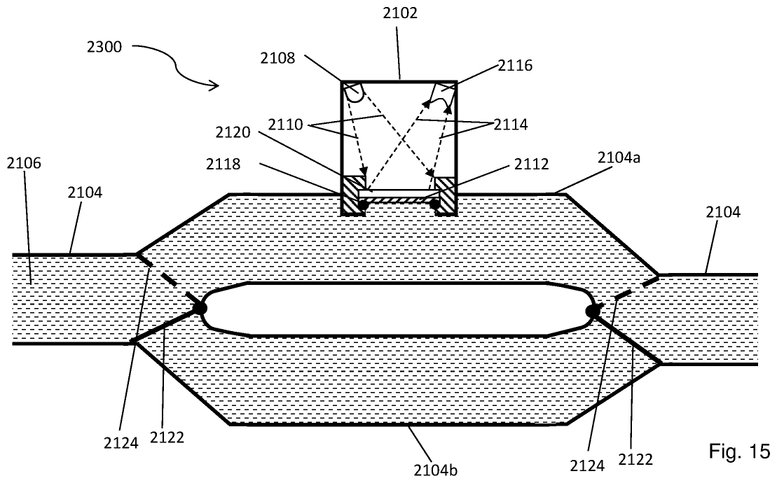

FIG. 15 shows a schematic of another example embodiment of an optical sensor for detecting and/or monitoring corrosion in a water system, having a replaceable metal sample;

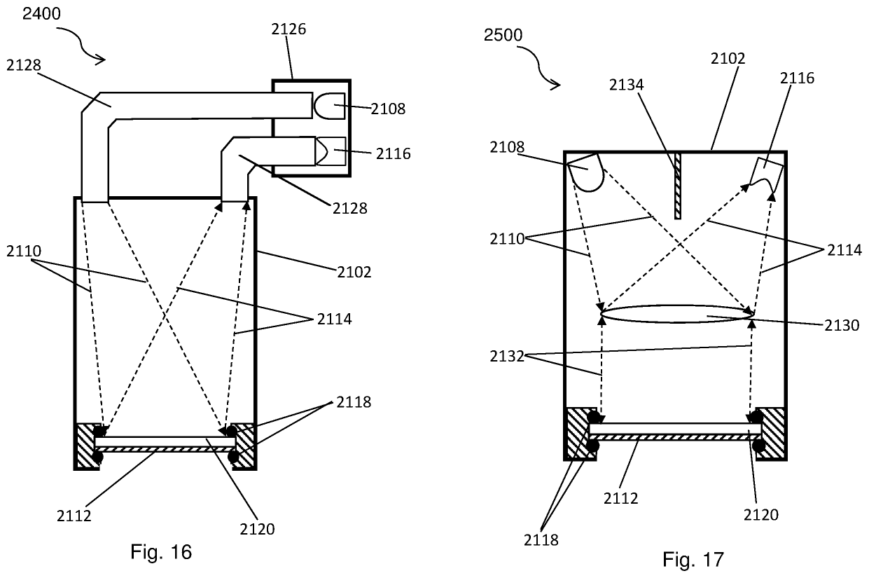

FIG. 16 shows a schematic of another example embodiment of an optical sensor for detecting and/or monitoring corrosion in a water system, having a light source and light sensor housed in a separate unit;

FIG. 17 shows a schematic of another example embodiment of an optical sensor for detecting and/or monitoring corrosion in a water system, having a baffle between the light source and the light sensor;

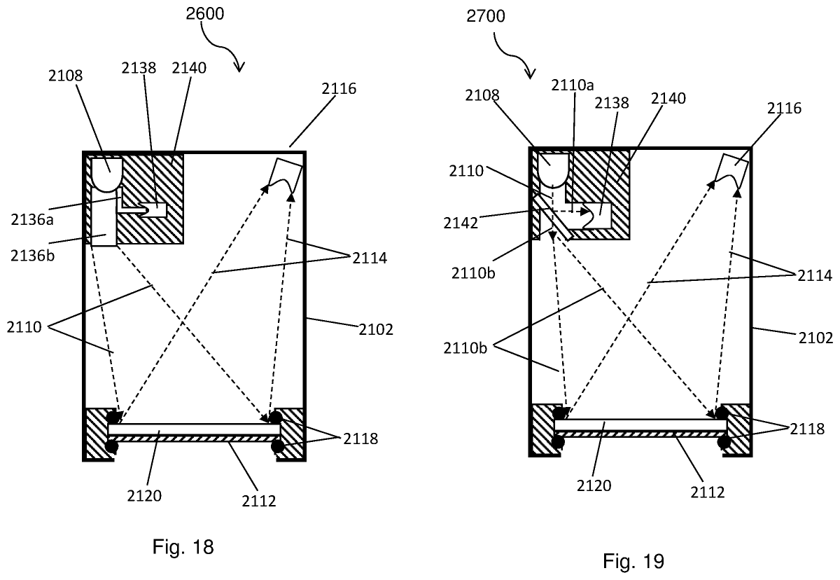

FIG. 18 shows a schematic of an example embodiment of an optical sensor for detecting and/or monitoring corrosion in a water system, having a second light sensor for directly sampling light emitted by the light source;

FIG. 19 shows a schematic of another example embodiment of an optical sensor for detecting and/or monitoring corrosion in a water system, having a second light sensor for directly sampling light emitted by the light source;

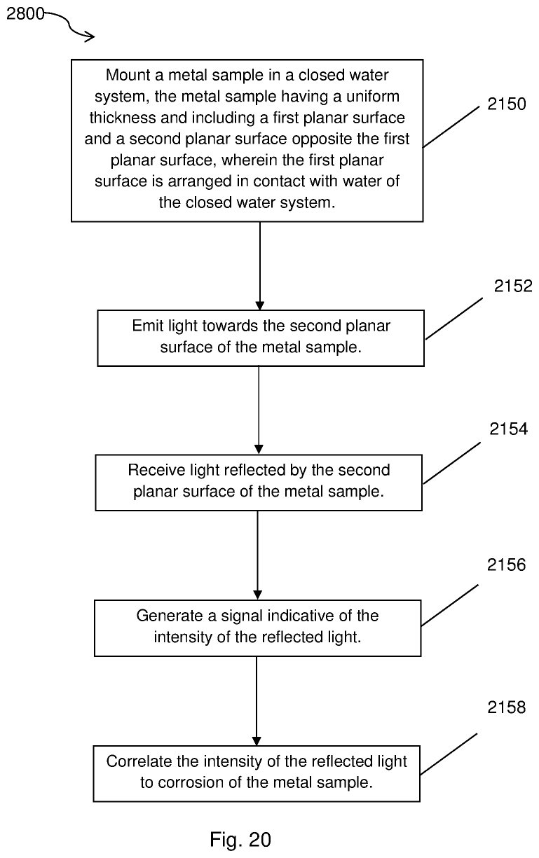

FIG. 20 shows a flow chart illustrating an exemplary method according to the present disclosure;

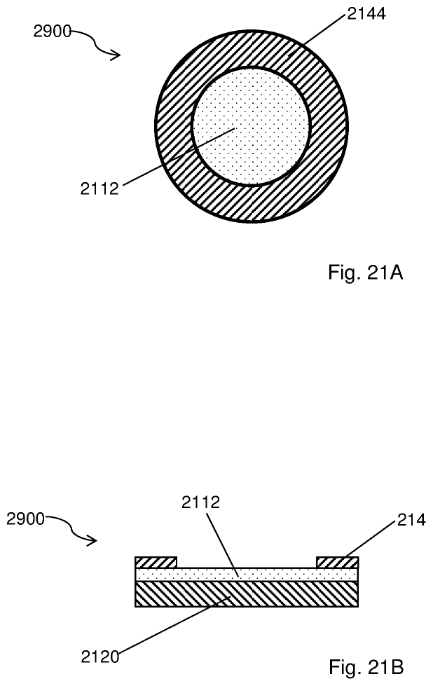

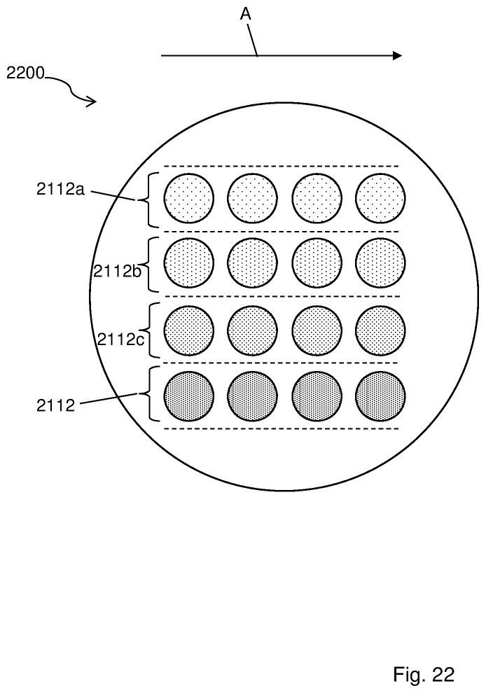

FIG. 21A shows a schematic of a plan view of a metal sample for use in the sensor;

FIG. 21B shows a side view of the metal sample for use in the sensor; and

FIG. 22 shows a schematic of an arrangement of multiple metal samples in a sensing apparatus.

The Figures will now be described in more detail. In each case, similar elements are labelled with the same number. The devices presented operate along the same broad principles, and consequently the overall operation will not be described in detail in each case. Instead, the differences between each Figure will be emphasised, and it is to be understood that operational principles are generally transferrable between each Figure, except where this would cause a contradiction.

FIG. 1 shows a flowchart representing the method disclosed. The first step 101 comprises receiving, from a first sensor, a value of a first parameter selected from the plurality of parameters. The plurality of parameters is based on at least one of the following: pressure; make-up water flow rate;

dissolved oxygen; cumulative dissolved oxygen; inhibitor dosing levels; biofilm accumulation; temperature; conductivity; galvanic current; cumulative galvanic current; crevice corrosion rate; and/or pH. This may involve measuring the value directly, or obtaining it through proxy means and inferring the result. The second step 102 comprises comparing the received value of the first parameter to a threshold range for the first parameter. The third step 103 comprises providing a diagnosis of a corrosion state based on the comparison of the value of the first parameter to the threshold range for the first parameter. The fourth step 104 comprises receiving, from a further sensor, a value of a further parameter selected from the plurality of parameters. This may involve measuring the value directly, or obtaining it through proxy means and inferring the result. As discussed above, this further parameter provides a more targeted approach to diagnose system abnormalities. The fifth step 105 comprises comparing the received value of the further parameter to a threshold range for the further parameter. The sixth step 106 comprises refining the diagnosis of the corrosion state based on the comparison of the value of the further parameter to the corresponding threshold range.

FIG. 2 shows an example embodiment of the method involving pressure as a determined parameter, and an example process for if the pressure was too low. In this scenario, first step 201 involves receiving, from a pressure sensor, the pressure of the system. The step 202 involves comparing the value obtained from the pressure measurement to a predefined threshold range of acceptable pressure values. If the measured value of the pressure is outside the threshold range, step 203 occurs. Step 203 involves providing a diagnosis based on the comparison. The diagnosis may comprise that the pressure is outside the threshold range, and in this case further comprising that it is too low, and below the lower limit of the threshold range. However, if the measured value is within the threshold range, step 203a occurs. Step 203a involves providing a diagnosis that the pressure is normal and within the threshold region. If the method takes this branch, then the system is behaving as normal. However, following this, further measurements of the same or different parameters may be taken in order to verify that the system is behaving normally.

In the instance that the pressure is too low, air may be drawn into the system, possibly eventually causing corrosion. At this point, an alert/message may be sent to the user that the pressure is too low, and optionally that further checks may be made to check for any signs of potential corrosion. To check if this is the case, further parameters may be measured in order to refine the diagnosis. If the pressure is too low, the process proceeds to step 204, where a further parameter value is received, in this case the dissolved oxygen level. Step 205 compares the dissolved oxygen level with a threshold range of acceptable dissolved oxygen levels. If the dissolved oxygen level is outside the threshold range, then step 206 occurs. Step 206 involves refining the diagnosis by determining that the dissolved oxygen levels are higher than the upper limit of the threshold range. If the measured dissolved oxygen level is within the threshold range, step 206a occurs in refining the diagnosis and stating that the oxygen levels are normal. In this case, the pressure may be checked again to ensure that it wasn't an anomalous result, and the dissolved oxygen levels may be monitored and measured again at a later time or at regular time intervals to check for any increase. Optionally, other parameters may be measured to ensure the diagnosis is correct.

If the oxygen levels are too high, then this suggests that air has been drawn into the system as a result of the system pressure being too low. This may lead to corrosion if left unattended. At this point, a further alarm/message may be sent to the user to bring it to their attention that the diagnosis has been confirmed. Optionally, other parameters may then be determined in order to further verify the diagnosis, or take a measurement of the rate of corrosion. Corrective action to move each parameter value back to within the threshold range may occur at any stage, preferably after the corresponding diagnosis is provided. For example, the pressure may be adjusted to within the threshold range after the diagnosis of the pressure being too low. Optionally, this may occur at another stage after further diagnosis.

For example, the method may progress to step 207 where a value of the galvanic current is received. This is then compared to a threshold range in step 208. Following this, if the measured value of the galvanic current is outside the threshold range, then step 209 occurs in refining the diagnosis and confirming that corrosion is taking place. However, if the galvanic current is within the threshold range, then step 209a in refining the diagnosis in that no corrosion has taken place yet is performed. Following step 209a, values of other parameters may be received to further determine if corrosion is taking place. If step 209 takes place, further parameters may also be measured such as crevice corrosion rate.

This embodiment provides an example of how a low pressure state can be detected and corrected as a preventative method to stop corrosion. The combination of measurements allows the system to identify a low pressure state which has led to air being drawn into the system, and may provide a diagnosis that corrosion is likely to occur before it has happened. This holistic overview of the system health can be used to take preventative corrective action.

This example also shows how the method can be used in a reverse sequence to identify the cause of a positive corrosion state instead of confirming corrosion. For example, if dissolved oxygen levels are above the threshold range, then the further parameter may be pressure to check if the pressure is too low, causing air ingress. Depending on this comparison, the diagnosis suggests a potential cause of the positive corrosion state. For example, if the pressure is normal, then the refined diagnosis may suggest that there may be a leak. Receiving a value of the make-up water flow rate may help refine the diagnosis and narrow down the corrosion cause.

FIG. 3 is another example embodiment showing an example process of detecting a leak in a closed water system. In this example, first step 301 involves receiving a value of the make-up water flow rate which could be measured by a water meter or flow sensor on the water make-up line. Step 302 compares the measured make-up water flow rate with a threshold range. If the value is outside the threshold range, and make-up water is being drawn into the system, then step 303 occurs. Step 303 involves providing a diagnosis that make-up water is being drawn into the system, which may be due to a leak in the closed water system, causing the system to be topped up with make-up water. This make-up water may be aerated, which may bring oxygen into the water system, and consequently lead to corrosion. However, if the measured make-up water is within the threshold range, then a substantial amount of make-up water has not been drawn into the system, and the diagnosis can be provided to state that the flow rate is normal during step 303a. In this example, the flow rate may be measured again at a later stage, along with other parameters.

If the water make-up flow rate is too high, the process proceeds to step 304, where a value of a further parameter is received, in this case the dissolved oxygen level in order to establish if the make-up water has brought oxygen into the system which in turn may lead to corrosion. It can also be used to further verify that make-up water has been drawn into the system. Step 305 compares the measured dissolved oxygen level with a threshold range of acceptable dissolved oxygen levels. If the dissolved oxygen level is outside the threshold range, then step 306 occurs. Step 306 involves refining the diagnosis by determining that the dissolved oxygen levels are higher than the upper limit of the threshold range. If the measured dissolved oxygen level is within the threshold range, step 306a occurs in refining the diagnosis and stating that the oxygen levels are normal. In this case, the make-up water flow may be checked again to ensure that it wasn't an anomalous result, and the dissolved oxygen levels may be monitored and further values used at a later time or at regular time intervals to check for any increase. Optionally, other parameters may be measured to ensure the diagnosis is correct. This checking process may be carried out at any step that involves providing a diagnosis or updating the diagnosis such as correcting or confirming the diagnosis.

If the oxygen levels are too high, then this suggests that aerated make-up water has been drawn into the system as a result of a leak in the system. This may lead to corrosion if left unattended, while the leak may have disastrous consequences depending on the function and location of the system. At this point, a further alarm/message may be sent to the user to bring it to their attention that the diagnosis has been confirmed and a leak may be occurring. Optionally, additional parameters may then be determined in order to further verify the diagnosis, or take a measurement of the rate of corrosion.

For example, the method may progress to step 307 where values of galvanic current are received from a galvanic current sensor. This is then compared to a threshold range in step 308. Following this, if the measured valued of the galvanic current is outside the threshold range, then step 309 occurs in refining the diagnosis and confirming that corrosion is taking place. However, if the galvanic current is within the threshold range, then step 309a in refining the diagnosis in that no corrosion has taken place yet is performed. Following step 309a, values of further parameters may be used to further determine if corrosion is taking place. If step 309 takes place, further parameters may also be measured such as crevice corrosion rate.

FIG. 4 is another example embodiment of the method disclosed, showing an instance where the pressure may be too high. This embodiment comprises steps that have previously been described, but are used to determine a different problem. The holistic overview of using measurements of set parameters can be used to identify the root cause of a problem. For example, in this case all the parameters of FIG. 3 are measured, and if they are all outside the threshold, the cause may be that a leak has occurred in the closed water system. However, by measuring the pressure, it may be determined that a high pressure has led to water loss through automatic air vents or other components, causing make-up water to be drawn into the system and consequent increase in dissolved oxygen.

In this example, first step 401 involves receiving a value of the pressure of the system. The step 402 involves comparing the value obtained from the pressure measurement to a predefined threshold range of acceptable pressure values. If the measured value of the pressure is outside the threshold range, step 403 occurs. Step 403 involves providing a diagnosis based on the comparison. The diagnosis may comprise that the pressure is outside the threshold range, and in this case further comprising that it is too high, and above the upper limit of the threshold range. However, if the measured value is within the threshold range, step 403a occurs. Step 403a involves providing a diagnosis that the pressure is normal and within the threshold region. If the method takes this branch, then the system is behaving as normal. However, following this, further measurements of the same or different parameters may be taken in order to verify that the system is behaving normally.

In the example that the pressure is too high, water may be forced out through automatic air vents (AAVs), pressure relief valves (PRVs) or other components, leading to aerated make-up water being drawn into the system, possibly eventually leading corrosion. At this point, an alert/message may be sent to the user that the pressure is too high, and optionally that further checks may be made to check for any signs of potential corrosion. To check if this is the case, further parameters may be measured in order to refine the diagnosis. If the pressure is too high, the process proceeds to step 404, where values of a further parameter are used—in this case the make-up water flow which could be measured by a water meter or flow sensor on the water make-up line. Step 405 compares the measured make-up water flow rate with threshold range. If the value is outside the threshold range, and make-up water is being drawn into the system, then step 406 occurs. Step 406 involves refining the diagnosis, confirming that the pressure is too high and has caused make-up water to be drawn in due to system water loss through automatic air vents. However, if the measured make-up water is within the threshold range, then a substantial amount of make-up water has not been drawn into the system, and the diagnosis can be refined correspondingly during step 406a. In this example, the flow rate may be measured again at a later stage, along with other parameters in order to ensure that the high pressure does not cause an uptake of make-up water at a later stage. A corrective action such as adjusting the pressure to within the threshold range may be taken.

If the water make-up flow rate is above a threshold (in some examples greater than zero), the process proceeds to step 407, where values of an additional, third parameter are received—in this case the dissolved oxygen level in order to establish if the make-up water has brought oxygen into the system which in turn may lead to corrosion. It can also be used to further verify that make-up water has been drawn into the system. Step 408 compares the measured dissolved oxygen level with a threshold range of acceptable dissolved oxygen levels. If the dissolved oxygen level is outside the threshold range, then step 409 occurs. Step 409 involves refining the diagnosis by determining that the dissolved oxygen levels are higher than the upper limit of the threshold range. If the measured dissolved oxygen level is within the threshold range, step 409a occurs in refining the diagnosis and stating that the oxygen levels are normal. In this case, the pressure may be checked again to ensure that it wasn't an anomalous result, and the dissolved oxygen levels may be monitored and measured again at a later time or at regular time intervals to check for any increase. Optionally, other parameters may be measured to ensure the diagnosis is correct. This checking process may be carried out at any step that involves providing a diagnosis or refining the diagnosis such as correcting or confirming the diagnosis, confirming the cause, or re-assessing the cause of the positive corrosion state.

If the oxygen levels are too high, then this suggests that aerated make-up water (i.e. fresh water having more dissolved oxygen than is desirable for system water) has been drawn into the system as a result of the system pressure being too high causing water loss through automatic air vents, pressure relief valves, or other components. This may lead to corrosion if left unattended. At this point, a further alarm/message may be sent to the user to bring it to their attention that the diagnosis has been confirmed. Optionally, further parameters may then be determined in order to further verify the diagnosis, or take a measurement of the rate of corrosion.

For example, the method may progress to step 410 where values of the galvanic current are received. This is then compared to a threshold range in step 411. Following this, if the measured valued of the galvanic current is outside the threshold range, then step 412 occurs in refining the diagnosis and confirming that corrosion is taking place. However, if the galvanic current is within the threshold range, then step 412a in correcting the diagnosis in that no corrosion has taken place yet is performed. Following step 412a, further parameters may be measured to further determine if corrosion is taking place. If step 411 takes place, further parameters may also be measured such as crevice corrosion rate.

By measuring certain parameters, the root cause of a system positive corrosion state can be identified, which may be rectified before corrosion takes place. The thresholds may be adjusted to new maintenance thresholds wherein the maintenance thresholds represent new limits within which normal operation of the maintenance event occurs. For example a parameter may be outside the normal threshold for a positive corrosion state during a maintenance event such as the dissolved oxygen concentration rising sharply during a mains water flush. However, this may be expected during the maintenance event, and hence a new maintenance threshold is used. If a parameter is outside this maintenance limit then there is an issue with the maintenance event being carried out, and the user can be alerted in the usual manner for corrosion detection mode.

FIG. 5 shows a flowchart of an example embodiment of the method disclosed, used for the assessment of a planned maintenance event. For example, if the system is configured in a maintenance mode, and the system is expecting specific maintenance events to occur, then this allows the diagnosis to confirm that the event has been successful by monitoring certain parameters. For example, if the system is expecting a water flush event 501, then values of the dissolved oxygen levels may be received in step 502. If there is a steep rise in dissolved oxygen levels 503 then this can be used to confirm that the water flush has occurred. This maintenance event may then be labelled on a graph displaying the monitored dissolved oxygen levels. Values of other parameters such as pH may be used such as in step 504 in order to confirm the water flush. Other parameters such as conductivity in step 506 may also be performed to further validate the measurements of other parameters in determining a maintenance event. However, in some embodiments only two measurements may be used to provide the diagnosis that an event such as a water event has occurred. Clearly it is preferable to monitor several parameters to provide a more detailed overview of the system, and multiple parameters can be used to inform the user about changing conditions which may be indicative of various events.

If a change in pH in step 505 is detected at the same time as the dissolved oxygen increased, this further confirms the diagnosis that the water flush occurred. For example, this may be as a result of the parameter (e.g. pH) reaching an expected value. In addition, if a change in conductivity is detected in step 507, this also indicates a water flush in combination with the measurements of other parameters. The values obtained and associated changes in parameters can be used to inform the diagnosis in step 508 which may be that a water flush has occurred. This can also comprise informing the user of a suspected water flush occurring.

Furthermore, the overall time of the water flush can be determined by recording the time of the deviation from normal. This can be performed by an additional step after diagnosis 508, which is not shown in FIG. 5. For example, the flowchart may comprise determining the duration of the water flush. Upon a comparison of this time to a required pre-set time for a mains water flush to occur successfully, this will confirm or otherwise that the water flush was carried out for the required length of time.

Further parameters may be monitored that are not shown in FIG. 5. In other examples, fewer than three parameters may be monitored and used to diagnose the maintenance event.

Further maintenance events may be monitored in this way. For example, in the pre-commissioning process it is typical that inhibitor may be added in step 509 shortly after the mains water flush occurs. This can be confirmed by receiving further values of conductivity after addition of inhibitor in step 510 and observing a sudden increase at 511. This may be further confirmed by receiving values of the pH in 512 and observing the increase due to the water flush eventually plateauing in step 513. These indications allow the diagnosis 514 to confirm that the inhibitor has been successfully added. Of course, if the expected changes to parameters are not detected, this can indicate that each maintenance event has not occurred successfully, and an alert may be sent to the user.

Further parameters may be monitored to ensure that the corrosion inhibitor is being effective. For example, the galvanic current or conductivity may be monitored to observe the levels of inhibitor and ensure it is effective in suppressing corrosion. Other maintenance events may be monitored in this way such as dynamic flushing, observing a cleaning chemical added to the water system, and draining the system. In some cases, this may be used to identify unplanned maintenance events, which may also be detected even if the system is not configured in the maintenance mode. For example, it may detect errors such as equipment failure or unplanned events such as a heating event that should not be occurring.

FIG. 6 is a flowchart showing a method of identifying or confirming a maintenance event using measurements of multiple parameters. For example, given a detected rise in a parameter such as dissolved oxygen at 601, values of various parameters may be used to determine what has caused this sudden rise. For example, values of the conductivity may be received at 602, wherein if a change is detected then this is indicative of a mains water flush event (for example flushing with mains water usually will result in a decrease in conductivity due to the aerated water). Other parameters can be measured to confirm this, for example the pH may be used at 604, wherein if the pH changes to an expected value at 605, this is also indicative of a mains water flush event. For example, the pH will increase if the water flush follows an acid clean, but will decrease if it follows normal operation in which alkaline inhibitors have been used. Furthermore, values of the flow rate may be used at 606, wherein if a rise in flow rate is also detected it indicates a mains water flush. By using multiple parameters the origin of the detected change 601 can be identified. In this case the diagnosis 608 may comprise that a mains water flush event has occurred. This process can be used to further verify that a particular maintenance event has occurred.

FIG. 7 is a graph showing an example of monitoring dissolved oxygen levels during maintenance events. The dissolved oxygen levels are shown in parts per million (PPM), while the measurements are shown over a period of 10 days. In other cases, the dissolved oxygen may be expressed in other units such as mg/L. The threshold upper limit for normal operation can be seen at approximately 0.5 PPM, an example threshold for this example. Above this value, a positive corrosion state occurs which may lead to corrosion due to high oxygen levels. Under normal circumstances the oxygen levels would ideally be kept below this. However, during a maintenance event the levels may far exceed this threshold. As such, a new maintenance threshold is required to ensure the smooth undertaking of each maintenance event. This can be seen at 10 PPM, where the maintenance threshold upper limit is shown. This is an example limit that may allow the system to monitor the success of maintenance events without unduly triggering a warning to a user that the normal system parameters have been exceeded.

Correspondingly, if the value exceeds this parameter, this is indicative of a positive corrosion state during a maintenance event, or some other malfunction. Since dissolved oxygen tends to saturate at the maximum possible value (e.g. around 10 PPM at 20° C. and 1 bar pressure), the maintenance mode threshold can be set around this level, so as to suppress alarm signals relating to positive corrosion states, when such saturation events occur. In the unlikely event that the dissolved oxygen nevertheless exceeds this value, an alarm mechanism exists to detect a malfunction (inlet water too aerated, wrong temperature, sensor malfunction, pressure too high, air ingress into the system, etc.). In some cases, the cumulative dissolved oxygen may be monitored, and a separate threshold will be provided for this. Therefore, during an event such as a water flush, if the cumulative dissolved oxygen exceeds a pre-set threshold, then an alert may be triggered. This may be useful to monitor the total amount of oxygen in the system, especially in situations where the instantaneous threshold will not be exceeded unless in a malfunction. For instance, if a big spike is detected, but this is brought under control in a certain length of time e.g. 1 day, then the cumulative dissolved oxygen may not exceed its threshold.

After dynamic flushing at day 0, a chemical cleaner is added which causes a dramatic drop in the dissolved oxygen down to 0 PPM. On day 1 a mains water flush occurs as the dissolved oxygen rises sharply up to approximately 9 PPM. After no more fresh water is being added to the system, the dissolved oxygen drops down to 0 PPM due to oxygen scavenging events taking place, but this occurs over a time interval of approximately 1 day. Also at the time when fresh water is no longer being added to the system, inhibitor is added, which may be detected by changes in conductivity and pH (seen in FIG. 8). After day 2, the oxygen level is back to approximately 0 PPM. Accordingly, the new maintenance threshold may comprise an upper limit of around 10 PPM, as shown in FIG. 7, meaning that if the dissolved oxygen increases up to 9 PPM as in FIG. 7, then the system is still behaving normally. However, if the level increases above 10 PPM, a positive corrosion state is triggered and the same procedure may occur as previously described when the system is in corrosion detection mode. When the parameter measurement reaches a certain value, it may be used to trigger an alert to perform the next event, or in some cases the next event may automatically occur. For example, if the dissolved oxygen level reaches 9 PPM, a message alert may appear containing information that the inhibitor should be added to passivate metal surfaces in light of the elevated dissolved oxygen levels, and/or scavengers may be added to reduce the oxygen levels in the system water. In another example, the inhibitor may be automatically added to the system when the dissolved oxygen level reaches a certain value e.g. 9 PPM. For this event, it may be appropriate to monitor other parameters such as conductivity and pH, and provide maintenance thresholds on these parameters in order to better provide an alert system.

In this example, the dissolved oxygen levels increase due to a mains water flush bringing aerated water into the system. The dissolved oxygen levels of the inlet water drawn into the system may be known, for example this may be tap water. As such the maintenance mode threshold range may be adjusted based on this. Therefore the threshold limit would not be exceeded for normal operation of this planned maintenance event.

FIG. 8 is another graph showing the pH levels over time for the same event sequence as FIG. 7. The pH drops when an acidic cleaner is added. The pH then increases and plateaus as the cleaner is consumed. During the mains water flush the pH rises sharply as residual acid from the cleaner is removed. When inhibitor is added, the rise begins to plateau around pH 9.5. Some example threshold limits have been indicated on FIG. 8. For example, the upper and lower threshold limits for normal operation are shown at pH 8.5 and 6.5 respectively. Under normal operation, the pH is desired to be within these limits to prevent corrosion. However, clearly in this instance, when a mains water flush occurs, the pH exceeds the normal upper threshold limit of pH 8.5. Accordingly, the limit is adjustable such that a maintenance upper limit can be provided to prevent an alarm being triggered when the pH exceeds the normal upper limit. For example, instead of an alarm being triggered, and the potential for automatic corrective action, the limit can be used as an indication that the event is occurring as planned. In addition, the maintenance threshold upper limit may be used to ensure that the pH does not deviate from that expected during the maintenance event. For example, this is shown as pH 10 in FIG. 8. Correspondingly, the lower limits are shown for normal and maintenance conditions, wherein these would work in a corresponding way as the upper limits.

These graphs in FIGS. 7 and 8 can be used to explain the processes of FIGS. 5 and 6, where events can be identified from the measurements of various parameters, and can be labelled accordingly.

FIG. 9 is a drawing showing an example embodiment of the apparatus for detecting corrosion in a closed water system disclosed. It comprises an inlet 901 through which water in a closed water system can enter a measuring unit 902. The measuring unit 902 allows the flow of water from the inlet 901 to the outlet 903. The measuring unit 902 comprises a plurality of sensors 904. In this embodiment, a first sensor 904a, a second sensor 904b, a third sensor 904c, and one additional sensor 904d are shown connected to the measuring unit 902. The number of sensors may be different to this, for example the apparatus may comprise more or fewer than shown in FIG. 9, which is only shown as an example embodiment of the apparatus. Each of the sensors is configured to determine values of the corresponding parameter selected from the plurality of parameters. For example, the first sensor is configured to determine values of a first parameter.

The measuring unit 902 is illustrative of a device to mount and position the sensors 904 in such a way that they intersect the water flowing from inlet 901 to outlet 903. It is also provided to ensure an air-tight seal such that the sensors 904 can monitor the parameters while preventing oxygen from entering the closed system, which allows representative measurements to be taken. In other examples, the measuring unit 902 may not be present, and the sensors may be directly connected to the water flow. A connection 905 is provided to allow transfer of data from the sensors 904 to a monitoring station 906. This connection may be an electrical connection, or may be fibre optic. The monitoring station 906 may comprise the processor to receive data from the sensors and interpret this data. For example, this processor may be an FPGA or may be a PC located locally or remotely. The monitoring station 906 may comprise the memory for storing a threshold range for each of the first, second, and third parameters, and in this embodiment the threshold range corresponding to the additional parameter as well. However, the memory may be located elsewhere, separate from the monitoring station and separate from the processor. It may further comprise a data logger or data recording means.

Alternatively, the processing may be performed elsewhere, and the data may be transferred from the monitoring station 906 to a processor in a remote location. For example, this may occur via an internet connection. In some cases it may be uploaded to the cloud. In the embodiment in FIG. 9, the monitoring station 906 comprises a display screen 907. In some embodiments, a display screen is not located adjacent to the sensors, and instead the data is transferred for example to a remote screen or accessible via a web interface, for user convenience. In some embodiments, the monitoring station may be connected to a wireless network, to which it may upload sensor data, for example to a web interface. The display screen 907 may display the sensor readings from sensors 904 in real-time or it may display information relating to how one or more sensor measurements change over time. This screen may be interactive in order for a user to obtain details on measurements from each sensor.

More additional sensors may be present in the apparatus and the processor may be configured to receive data from each of these additional sensors. A sensor (not shown in FIG. 9) may be included between the inlet 901 and the outlet 903, for example the one described in more detail below.

Although not shown in FIG. 9, the apparatus may comprise a user input for manually selecting the parameters from the plurality of parameters. The user input may also adjust the apparatus configuration from corrosion detection mode to maintenance mode and vice versa. For example this may comprise a switch or interface such as a keyboard or touchpad.

Although not shown in FIG. 9, the apparatus may comprise means for adjusting a parameter such as control of a pressurisation unit, control of water flow rate, control of make-up water flow rate, control of automatic air vents or pressure relief valves, addition of corrosion inhibitor, addition of an anti-biofilm agent, a heating and/or cooling unit, and/or pH control. These elements may be present within measuring unit 902, or may exist at specific points along the water flow path, depending on the requirements of the means to adjust each parameter.

The measuring unit 902 allows the sensors 904 to be immersed in the water flowing from inlet 901 to outlet 903 whilst not interrupting the flow. In other words, the flow is continuous and measurement of the sensors 904 does not require a disruption to the flow and samples taken of the water. This ensures the water being measured has not become aerated during the process, unlike many previous systems. In some embodiments, the sensors 904 may be distributed around the system, or a plurality of monitoring stations 906 may be present at different locations around the system. As described above the sensors 904 may comprise means for measuring one or more parameters such as pressure; make-up water flow rate; dissolved oxygen; cumulative dissolved oxygen; inhibitor dosing levels; biofilm accumulation; temperature; conductivity; galvanic current; cumulative galvanic current; crevice corrosion rate; and/or pH. Although four sensors are shown in FIG. 9, the system may comprise more than four, or fewer than four. In some examples, fewer than all of the sensors may be actively monitoring and outputting data concurrently. In some cases, as few as two sensors may be present or actively monitoring and outputting data. In other cases, all the sensors present are actively monitoring data at the same time.

Some sensors may not require immersion in water, such as temperature. The measuring unit 902 provides a means for positioning each sensor in an appropriate location to perform the determining of the corresponding parameter. For example, in some cases the thermal conductivity of pipes (particularly copper pipes) may be generally high enough such that the measurement of the temperature of the exterior of the pipe is a good approximation to the temperature of the water within the pipe.

FIG. 10 shows an example embodiment of the sensor described above. Inlet 1001 is for receiving water from a closed water system, for example a heating, ventilation and air conditioning systems, allowing the water to pass through the sensor to outlet 1002, where the water is returned to the closed water system. The pipes connecting the sensor to the closed water system, such as at the inlet 1001 and outlet 1002, may be for example made of copper or other metals typically used in such systems. The sensor comprises a sensing chamber 1003, located between the inlet 1001 and the outlet 1002, such that the water passes through the sensing chamber. The sensing chamber comprises an outer chamber wall for retaining water in the sensing chamber. The sensing chamber also comprises a first measurement surface 1004 formed from a first metal. For example the first metal may be copper. The sensor also comprises a second measurement surface 1005 mounted at least partly within the sensing chamber, and formed from a second metal, the second metal being different from the first metal. For example, the second metal may be steel. In other examples the first and/or second metals are selected from: brass, steel, copper and alloys thereof or other metals or alloys typically used in such systems, wherein the first and second metals are different. In the example shown in FIG. 10, the first sensor 1004 is the outer chamber wall. The second measurement surface 1005 is typically chosen to be the anode by selecting a metal lower (i.e. less noble) in the galvanic series than the first measurement surface 1004. This prolongs the life of the sensor, since it will be the less noble metal which corrodes, and consequently (in the design shown) the first measurement surface 1004, which doubles as the outer wall) will not be corroded, thereby reducing the likelihood of leaks. The second surface can be replaced if needed, either to change the metal, to provide information on the corrosion of a different metal/alloy, or to replace a corroded inner measurement surface. In any case the metal/alloy which will be the anode is advantageously made from a metal which is representative of metals in the closed water system to which the sensor is connected. In some cases, a variety of versions of the sensor having anodes made from different metals may be provided for use in different systems having predominantly those metals exposed to water.

The sensor is configured such that water flowing in through the inlet 1001 flows through region 1006 between the inner surface of the first measurement surface 1004 and the outer surface of the second measurement surface 1005. For example, the first measurement surface 1004 may be a pipe of circular cross-section, and the second measurement surface 1005 may be a pipe or rod of circular cross-section, with a diameter smaller than the first element, such that the second measurement surface 1005 is disposed within the first measurement surface 1004. In this example, the water flows through annular region 1006 between the two surfaces.

The first and second measurement surfaces include electrical connection points for connecting to current sensing means 1008 (also known as current measuring device). The presence of the current measuring device 1008 means that a preferential flow path between the first 1004 and second 1006 measurement surfaces exists. This causes charge build-up on the measurement surfaces to flow though the current measuring device. The resulting current is measured, and an indication of the amount of corrosion occurring with respect to time is provided by the current measuring device 1008. The current measuring device 1008 in some cases has a local memory for storing the measured current as a function of time. In other cases, the current v time information is communicated elsewhere, for example as part of the overall system health monitoring described above. FIG. 10 shows the first and second measurement surfaces including electrical connection points 1007a and 1007b, respectively, for connecting to a current sensing means 1008 via an electrical connection 1009. In some cases, the electrical connection 1007 points may allow the current sensing means 1008 to be reversibly connected, e.g. for replacement. In other examples, the current sensing means 1008 may be permanently connected (e.g. hard-wired) to the sensor. For example, the current sensing means 1008 may be an ammeter, perhaps especially configured to measure small currents such as on the milliamp scale. In some examples, the current sensing means is configured to integrate the current over time.

As water flows over the two surfaces, galvanic corrosion of the anode occurs. This causes charge to build up on the surfaces. Since the surfaces are connected by an ammeter, the charge flows along this current path and the ammeter registers a signal, proportional to the rate of corrosion. The total amount of corrosion which has occurred can be derived from this by integration, as described above.

In some examples, the sensor is configured to send the output from the current sensing means to a processor. This processor may be the same processor of the apparatus for monitoring a plurality of parameters for detecting corrosion described above. In this case, the sensor may be one of the sensors of the apparatus.

In some examples, there are two measurement surfaces within the sensing chamber 1003, and the outer wall of the sensing chamber 1003 is not used as a measurement surface. In other respects such a sensor operates in much the same way as that described above. Although the measurement surfaces 1004, 1006 are shown as smooth, flat surfaces, corrugations, ridges, or other complex shapes may be used to increase the surface area.

The current sensing means 1008 can be configured to relate the current it is measuring to the degree of effectiveness of the inhibitors in the water system, as described in detail above. Additionally or alternatively, the current can be related to a rate of loss of thickness of exposed metal surfaces in the system, or the current can be integrated with respect to time and the result used to give a measure of the total loss of metal thickness in the system during the period over which the integration occurred. This can be done either as an integrated system, or by passing the current data to an external processor which performs the steps of integration and/or relating the current to a degree of effectiveness of the inhibitor or to the rate of loss or total loss of metal in the system. In some cases, the current sensing means 1008 may be configured to send alerts to a user that the measurement indicates a positive corrosion state (non-zero current), and suggest a corrective action such as addition of inhibitor. The sensor may even be configured to take corrective action automatically and/or to monitor the current while corrective action is being taken to iteratively arrive at the correct concentration of inhibitor to fully passivate exposed metal surfaces in the system.

Turning now to FIG. 11, an illustration of the effect of inhibitors in a closed water system is shown. More specifically, the graph 1100 demonstrates how the galvanic current in a system changes as inhibitor dosing levels change (as determined by measurement of conductivity), in this case at a fixed temperature of 60° C. This figure shows the effects in an oxygenated water system. As discussed in detail above, the galvanic current is a direct representation of the amount of corrosion occurring in the system. An arrow 1102 points towards low inhibitor concentrations. The conductivity of pure tap water (i.e. with no inhibitor added) is around 300 μS/cm in this example. However, the conductivity of mains water varies throughout the UK and is lower in soft water regions, and higher in hard water regions. As inhibitor is added to the water, the conductivity increases. At the full recommended dose of inhibitor, the conductivity reaches approximately 850 μS/cm.

It can be seen from this arrow 1102 that the conductivity and the concentration of inhibitor are correlated with one another. Moreover, the correlation is positive, in that low concentrations correspond to low conductivities and vice-versa. It is clear that if the correlation between the inhibitor concentration and the conductivity is known in advance, then the concentration can be directly related to the measured conductivity value to a reasonable degree of accuracy. This provides a convenient way of determining the concentration. Indeed, as is clear, seven data points have been measured, corresponding to seven inhibitor concentrations and their corresponding conductivity. These can be stored together, for example in a tabular format. The correspondence between these values can be used, e.g. to change the scale on the x-axis from conductivity to inhibitor concentration (e.g. in ppm, percentage [by volume or weight], percentage of recommended dose, etc.) for ease of reference by a user.

As noted above, the graph 1100 shows data at a constant temperature of 60° C. In some cases, each data point may be stored with a corresponding temperature at which that data point was measured. By storing temperature data in this way, the determination of concentration from conductivity can be improved, since the effect of temperature can be removed. In some cases, two or three of the parameters of conductivity, inhibitor concentration and temperature can be fit to a generalised equation with variable coefficients. The set of coefficients which gives the best fit to the data can be stored and used to determine correlations between conductivity and concentration where no data points exist. In some cases, the stored data can be boosted with further calibration measurements, further refining the accuracy of the fit. A different correlation may be determined for different inhibitor types, as set out above.

As shown in the graph 1100, the galvanic current is high at low inhibitor concentrations and vice-versa—see the stepped plot line 1104. This relationship is to be expected since the purpose of the inhibitor is to prevent or reduce corrosion. Therefore when there is no inhibitor, corrosion continues largely unchecked, and the corresponding galvanic current is high. As inhibitor approaches its recommended value, the current is reduced due to the effectiveness of the inhibitor. The stepped nature of the graph 1100 naturally leads to considering there to be two different regimes for the inhibitor. A first regime 1106 in which galvanic currents are high because the exposed surfaces of the metal in the system are not passivated, or at least the passivation is not completely effective. This spans approximately the range of conductivities between approximately 300 μS/cm and 650 μS/cm in this example.

The second regime 1108 ranges from approximately 650 μS/cm to 850 μS/cm. Here, the galvanic current plateaus at a low value and does not change much with further inhibitor being added to the system. This is a sign that the exposed metal surfaces of the system have been passivated effectively. Clearly the system should be operating in the second regime 1108 and not the first regime 1106, in order to minimise corrosion when dissolved oxygen is present.

Overall, the graph 1100 in FIG. 11 illustrates that the inhibitor concentration and the conductivity are inherently linked; that the temperature is an important effect to account for in the conversion between these two parameters; and that the effectiveness of the inhibitor in the system can be directly seen from the dramatic effect it has on galvanic currents.

As noted above, the correlation between the conductivity and the inhibitor concentration is important to determine accurately, so that the concentration can be determined simply and accurately. In FIG. 12, a flowchart 1200 of a method for determining the correlation and/or using a correlation so determined to convert between these two parameters. The method starts at a first step 1202, in which a correlation between conductivity and inhibitor concentration is stored. This may be by virtue of a system being provided with this information pre-installed, or there may be various calibration steps, e.g. adding inhibitor at a known concentration to the water in the system and measuring the conductivity. As noted above, this can include storing data in a tabular format or by reference to an equation with variable coefficients (in which the best fit coefficients are stored). Any of these calibration steps may be performed as often as necessary to ensure that adequate accuracy can be achieved. Additional readings taken at any time while the device is operating (and for any reason, e.g. calibration, or normal operation) can be added to the stored correlations, so that they may be retrieved in future. In some cases, more recent data overwrites older data.

Next, at step 1204, a value for the conductivity is determined. This is typically performed by a suitable sensor which forms part of the overall system. These measurements may be made continuously or periodically, for example. A central control and processing unit may be configured to control such a sensor, and request that a reading be taken at times when this is needed, for example.

At step 1206, the temperature of the water is determined, so that temperature effects can be accounted for. In some cases, the temperature may be determined before the conductivity in order for the conductivity measurement to be actively compensated. In other cases, the order is not important and the compensation is done at a later stage when the conductivity is related to the correlation. In part, the measurement of temperature may include using a heater or cooling device to hold the temperature constant (e.g. at a value for which data exists). Alternatively, different correlations may be stored which correspond to different temperatures. In some cases, the temperature measurements may be used to actively compensate for the temperature effects in the conductivity sensor. The correct correlation for the current temperature can be used, or where this is not available, the closest may be used (possibly including an extrapolation or interpolation).

At step 1208, the determined conductivity value is compared to the appropriate stored correlation. Next, at step 1210, the comparison of the conductivity to the inhibitor takes account of the temperature measurement. For example, the conductivity measurement may be compensated based on the temperature. Finally, in step 1212, the corresponding inhibitor concentration is provided based on the comparison. This provides a simple way to determine the inhibitor concentration (which is difficult to determine accurately) by use of a proxy measurement.

The strict order of the steps in FIG. 12 need not be adhered to. For example, the temperature may be measured prior to the conductivity being measured. In some cases, the conductivity sensor has a temperature-compensated output.

As will be clear, the specific sensing systems for the inhibitor concentration and galvanic currents provide improved sensing of some of the parameters which the general system uses to determine system health.

FIG. 13 shows an example embodiment of an optical sensor 2100 for detecting corrosion in a water system. The optical sensor 2100 comprises a sensor housing 2102, which, in use, is partially positioned within a water system. As shown, the sensor 2100 is mounted to a pipe 2104 of the system, which is filled with system water 2106. The sensor includes a metal sample 2112 (for example a thin film or foil of metal) in contact with the system water 2106. In particular, a first surface of the metal sample 2112 is in contact with the water 2106 of the water system. The contact between the first surface of the metal sample 2112 and the system water 2106 leads to corrosion of the metal sample 2112. As the metal sample 2112 corrodes, corrosion debris and/or tarnishing will appear on a second surface opposite surface to the first surface (and separated from the first surface by the thickness of the metal sample 2112), which will decrease the reflectivity of the surface. In some cases, the metal sample 2112 will corrode away entirely in parts, for example due to pinhole corrosion progressing through the entire thickness of the sample 2112, or due to uniform corrosion of such severity that the parts of the metal sample 2112 are entirely corroded away.

In some examples, and as shown in FIG. 13, the second planar surface of the metal sample 2112 is not arranged in contact with the system water 2106. In some examples, to ensure that pinhole corrosion occurs through the thickness of the metal sample 2112 from the first planar surface in contact with the system water 2106 towards the second planar surface, and not in the other direction, water is prevented from contacting the second planar surface.

The optical sensor 2100 is configured to illuminate the metal sample 2112 with a beam of light 2110 emitted from a light source 2108. The optical sensor 2100 is also provided with an optical sensor 2116 to receive the reflected light 2114 and determine the intensity of that reflected light beam 2114. The reflectivity of the metal sample 2112 can be determined from the intensity of the reflected light beam 2114 received, which is related to the amount of corrosion debris on the second surface of the metal sample 2112, tarnishing due to contact with the system water 2106 and outright missing metal. Consequently, the change in reflected light intensity is related to the amount (and in some cases, the type) of corrosion of the metal sample 2112. Note that the optical sensor 2116 can detect changes in reflected light in this way whether or not the reflection is diffuse or specular. In either case, the amount of reflected light changes and a change in received light intensity can be detected.

A common cause of corrosion in water system is dissolved oxygen within the system water 2106. This causes corrosion of metals within the water system, as described above. The metal sample 2112 is used as a sacrificial sample to monitor and/or detect corrosion. If the water 2106 in the water system 2102 is corrosive, for example it contains high levels of dissolved oxygen or other corrosive components (acids, bases, organic chemicals, etc.), then corrosion of the metal sample 2112 can occur. This may take the form of pinhole corrosion, also known as pitting corrosion, where pinhole-sized holes form in a metal. Due to surface irregularities and imperfections, a small pit forms in the first surface in contact with the water due to pitting corrosion. Pinhole corrosion is particularly troubling because it results in very little loss of metal, so is hard to assess severity using weight loss or cumulative galvanic current studies, yet it causes damage to the deep structure of metal, and can corrode holes entirely through metal (e.g. through pipe walls, causing leaking). Worse still, pinhole corrosion is often obscured by corrosion debris such as tubercles or tarnishing, meaning that it is often not clear whether a tarnished portion represents mere surface corrosion or if one or more pinholes have caused much deeper problems.

The sensor 2100 has an O-ring seal 2118 to prevent system water 2106 from entering the interior of the housing 2102. This seal also helps to ensure that the system water 2106 cannot contact the second surface of the metal sample 2112 by leaking around the outer edge of the metal sample 2112. The O-ring seal can be formed from any suitable material for making a watertight seal which can withstand the conditions found in the water system. For example, temperatures up to around 100° C., high pressures, and exposure times of 5 to 10 years. In addition, a transparent plate 2120 is provided on the second surface of the metal sample 2112, thereby ensuring that the system water 2106 cannot contact the second surface, other than by corroding through the metal sample 2112. The transparent plate 2120 can be formed from plastics, glasses, etc. and be suitable for withstanding the temperatures and pressures set out above for the timescales of intended use. Since the metal sample is relatively thin (often thinner than the thickness of pipes, etc. in order to provide advanced warning of a corrosion problem), it may not be sufficiently strong to withstand the pressures in the water system. The transparent plate 2120 can provide structural support to the metal sample 2112. The transparent plate 2120 does not affect the operation of the system, since (being transparent) light emitted 2110 from the light source 2108 travels through the transparent plate 2120, reflects from the metal sample 2112, travels back through the transparent plate 2120 and is received by the light sensor 2116. In any case, the sensor can be calibrated to ensure the range of output is indicative of 0% to 100% loss of material. In some cases, there may be no need for a transparent element 2120 in this location, for example, where the metal sample 2112 is strong enough to resist the system pressures, and where there is nothing on the interior of the housing 2102 which would be adversely affected by system water 2106 leaking into the housing 2102, as would be the case if the metal sample 2112 is penetrated by corrosion and there is no transparent element 2120.

Since only the first surface of the metal sample 2112 is exposed to the system water 2106, the only way for the second surface to corrode (i.e. for tarnishing or loss of metal to show on the second surface) is for the corrosion to travel through the whole thickness of metal. As shown, the metal sample 2112 has a uniform thickness, which means that detection of a pinhole corroding through the sample 2112 is not dependent on the location of the corrosion. In other words, pitting corrosion occurring anywhere on the metal sample 2112 must travel through the same thickness of metal in order to form a pinhole, and has the same effect on the reflected light beam 2114.

As the metal sample 2112 is chosen to be thin, pitting can form a small pinhole through the metal sample 2112. Corrosion debris spreads from pinholes on the second surface of the metal sample 2112. The corrosion debris decreases the reflectivity of the surface, which provides a detectable decrease in intensity of reflected light 2114.

The metal sample 2112 is chosen to be a metal typically found elsewhere in the water system 2102. Typically, the metal sample 2112 may be formed from carbon steel, copper, stainless steel, brass, or aluminium. By choosing a metal that is present in the water system, determining the amount of corrosion or rate of corrosion of that metal within the optical sensor 2100 provides an indication of the corrosion of that metal elsewhere in the system.

The metal sample 2112 is chosen to be a thin film such that corrosion occurs quicker than at sections of metal in the rest of the system 2102. This allows a fast diagnosis of corrosion without waiting for corrosion of parts of the actual system to progress to unsafe levels. This can prevent significant damage to the system, savings costs by identifying corrosion without waiting for catastrophic failure.

The metal sample 2112 is sealed by seals 2118 such that the first surface of the metal sample 2112 is exposed to the water while the second surface is not. The seals 2118 prevent water from leaking around the metal sample 2112 while it is held in position. For example, the seals 2118 may be in the form of rubber seals, or a glue/resin between the metal sample 2112 and the walls of the sensor housing 2102. The seals 2118 prevent the water from being in contact with the electronics and sensing apparatus, which will now be described.

The optical sensor 2100 comprises a light source, for example the light source 2108 in FIG. 13 may be a light-emitting diode (LED) 2108. The LED 2108 is configured to emit light onto the metal sample 2112. In particular, the LED 2108 is configured to emit light onto the second surface of the metal sample 2112. The second surface is the opposite surface to the surface in contact with the water. The emitted light is shown in FIG. 13 by arrows 2110. The metal sample 2112 is reflective, and therefore reflects the light from the LED 2108. The reflected light is shown by arrows 2114.

In other examples, the light source 2108 may be a laser, or a fluorescent light bulb or any other suitable light source.

The optical sensor 2100 also comprises a light sensor 2116, for example, the light sensor 2116 shown in FIG. 13 may be a photodetector. The photodetector 2116 is configured to detect the light emitted from the LED 2108. In particular, the photodetector 2116 is configured to detect the reflected light 2114 from the second surface of the metal sample 2112.

The photodetector 2116 is positioned adjacent to the LED 2108 such that light from the LED 2108 directed towards the metal sample 2112 (light beam 2110), which is then reflected (light beam 2114) by the second surface of the metal sample 2112, and is received by the photodetector 2116. The photodetector 2116 and the LED 2108 are on the same side of the metal sample 2112, facing the second surface of the metal sample 2112. This allows the light source and the light sensor to be isolated from the water within the water system 2102, for example by the housing 2102 and the seals 2118.

In some examples, the photodetector 2116 is configured to not detect light emitted directly from the LED 2108 that has not been reflected by the metal sample 2112, that is no direct light path between the LED 2108 and the photodetector 2116 exists. For example, an opaque barrier may be placed between the LED 2108 and the photodetector 2116 to prevent stray light affecting the readings, as set out in more detail with regard to FIG. 17. In other examples, the output of the photodetector 2116 photodiode is calibrated to take into account the inner optical properties of the cavity including the reflectivity of the metal sample 2112. In other words the photodetector 2116 may be calibrated to remove the effects of the direct LED light on the readings, and only detect changes due to changing reflectivity of the metal sample 2112. In yet further examples, the light source 2108 and light sensor 2116 may have relatively narrow angular ranges of emission and/or detection respectively, so that there is no direct transfer of light from the light source 2108 to the light sensor 2116.

The photodetector 2116 is configured to transmit a signal corresponding to the detected light reflected by the metal sample 2112. For example, the photodetector 2116 outputs a signal which is related to the intensity of the detected light, in the form of an analogue signal. For example a current or voltage signal with its amplitude related to the received intensity. In other examples the sensor 2100 may output a digital signal in a standardised format, such as Modbus.

The sensor 2100 may further comprise a processor (not shown), configured to receive the signal from the light sensor 2116 corresponding to the detected light reflected by the metal sample 2112. For example, the light sensor 2116 is configured to measure the intensity of the received light, and transmit the signal corresponding to the intensity towards the processor (which may be remote, for example for collating many measurements from a variety of sensors). The signal is transmitted to the processor via an electrical, wireless, fibre-optic, etc. connection.

The processor may comprise a data acquisition system for storing the received data from the light sensor 2116. For example, the data acquisition system may be a data logger or other memory controllable by the processor.

The processor may be configured to process the data received from the light sensor 2116. For example, the processor receives periodic measurements of the intensity from the light sensor 2116. The processor is configured to convert the measurement signal from the light sensor 2116 into intensity of light. The processor is configured to store the periodic intensity values in a table, and output to a user. For example, the data may be outputted to a display device for user interaction by displaying the table of values, or displaying a graph of the intensity values over time.

The processor is configured to detect changes in the values of intensity. For example, if the intensity of light decreases substantially over time, this may correspond to a decrease in the reflectivity of the metal sample 2112. This decrease in reflectivity is caused by corrosion of holes through the metal sample 2112 and/or subsequent spread of corrosion debris over the surface reducing the intensity of light reflected by the surface. This data may be extrapolated to estimate the time remaining until one or more pipes in the system corrode through and start to leak.

Upon detecting a decrease in intensity, the processor may be configured to trigger an alert. A threshold may be present to indicate corrosion of the metal sample 2112. For example, a decrease from an initial high value of intensity by a certain amount, below a threshold, may trigger an alert. In another example, the threshold may be related to the gradient of the decreasing intensity, or a percentage decrease from a highest value of intensity. If a threshold is exceeded, an alert such as a message to a user may be triggered.

The processor may be configured to control the light source 2108 and/or the light sensor 2116. For example, the intensity or spectral range of light emitted by the light source 2108 or the spectral range at which the light sensor 2116 is most sensitive may be controlled electronically to gain more information about the type and severity of corrosion in the system.

Consider now FIG. 14, which shows another example embodiment of an optical sensor, 2200 having a plurality of metal samples 2112. The sensor 2200 is similar to that shown in FIG. 13, but has three metal samples 2112a, 2112b, 2112c. Each of these is formed from the same metal in FIG. 14, and has a different (uniform) thickness. Each sample has its own respective light source 2108a, 2108b, 2108c and light sensor 2116a, 2116b, 2116c for detecting corrosion in the manner set out above. Since each metal sample 2112 has a different thickness, knowledge of which sample has corroded provides information on the depth of metal which has corroded in the system and also the depth of metal which has not corroded. For example, if the thinnest metal sample 2112a is 0.025 mm thick, with the next sample 2112b being 0.05 mm thick and the thickest sample 2112c being 0.075 mm thick, then if the first sample 2112a shows tarnishing or other signs of corrosion, while the other two 2112b, 2112c do not, then it can be inferred that the corrosion depth is between 0.025 mm and 0.05 mm. This determination can be performed locally using a processor (not shown), or the readings can be transmitted to a remote location for processing.

In some cases, instead of metals of different thicknesses, the multiple samples 2112 may be formed from different metals and/or alloys. This can provide a measure of how different metals present in the system are faring under exposure to system water 2106. Of course, in some cases, there may be a combination of different metals and different thicknesses to provide an estimate of the maximum pitting corrosion depth in several metals at once.

While each metal sample 2112 is shown with a corresponding light source 2108 and light sensor 2116, in some cases a single light source 2108 may be configured to shine light onto a plurality of metal samples 2112, and/or a single light sensor 2116 may be configured to receive light from a plurality of metal samples 2112. Thus, associating many samples 2112 with a single housing 2102 as shown in FIG. 14 may allow a reduction in the amount of electronics in the system, thereby saving costs.

The plurality of metal samples 2112 may be formed from the same metal as one another, wherein each of the plurality of metal samples 2112 has a different thickness to the thickness of the other metal samples 2112. This can allow corrosion of different types of metal to be monitored by providing samples 2112 of different metals. In other cases, a series of samples of different thicknesses may be provided. This can provide a more compact sensor than providing a separate sensor for each thickness of metal and/or each different metal type. Of course, an alternative arrangement is to provide a separate sensor 2100 such as that in FIG. 13 for each metal and/or thickness. Where a single sensor 2200 has a plurality of metal samples 2112, the sensor 2200 may share various measurement components. For example, a single light source 2108 (or in general fewer light sources 2108 than there are samples) can be arranged to shine onto the second surface of multiple metal samples 2112, thereby ensuring that each sample 2112 is illuminated with the same light, so allowing a meaningful comparison of the received light 2114 intensity, as the light received 2110 by the second surface of each sample 2112 can be controlled to be the same in each case. In other cases, there may be a single light sensor 2116 and multiple light sources 2108. For example, each light source 2108 may emit light 2110 in a relatively narrow wavelength range and the sensor 2116 may be configured to detect the received intensity broken down by received wavelength, thereby allowing the sensor 2116 to correlate intensity in a wavelength with a particular sample (wavelength division multiplexing), and thus monitor several samples 2112 using only one sensor 2116, or more generally using fewer sensors 2116 than there are samples. In yet another case, other aspects such as processors for interpreting the received intensity data or housings 2102 for enclosing the sensors 2116 may be shared between multiple metal samples 2112. An alternative method of sharing light sources 2108 and light sensors 2116 is to use a highly directed light source 2108 and/or light sensor 2116 and illuminate the second planar surface of different samples 2112 at different times (time division multiplexing), and synchronise the sensor 2116 with the light source 2108, so that light received 2114 at a given time is correlated with a given metal sample 2112. In yet further examples, baffles such as that shown in FIG. 17 (element 2134) may be used to prevent direct transmission from a light source 2108 to a light sensor 2116, and also to prevent transmission of light from one “sensing unit” (light source 2108, metal sample 2112 and light sensor 2116) to another sensing unit, i.e. to prevent cross talk between measurements on each sample 2112.

Turning now to FIG. 15, a further example sensor 2300 is shown, which has a bypass arrangement. The sensor 2300 operates in a broadly identical manner to that shown in FIG. 13. In this case, however, the sensor 2300 is mounted on a section of pipe 2104 having two flow paths in parallel. The main pipe 2104 branches into a sensor section 2104a having the sensor 2300 mounted to it, and a bypass section 2104b, having no sensor. System water 2106 is able to flow through the main pipe, and then either through the sensor section 2104a or the bypass section 2104b. The water 2106 is controlled by two valves, each having a first position 2122 where they block water from flowing into the bypass section 2104a and a second position 2124 where they block water from flowing into the sensor section 2104b. Thus, where water is intended to flow through the sensor section 2104a, the valves are each set to their first positions 2122, and water is blocked from flowing into the bypass section 2104b and instead flows past the sensor 2300 via the main section 2104a of pipe. Similarly, the water 2106 can be arranged to miss out the sensor section 2104a of pipe by diverting water 2106 through the bypass section 2104b or pipe, which is achieved by setting the valves to their second positions 2124. In some cases, the two valves can be connected such that triggering one valve to change between its first 2122 and second 2124 positions causes the other one to change. That is, rather than having each valve being independently controllable (leading to four distinct configurations, two of which are effectively to block flow through the element entirely), there are simply two configurations: bypass and sensor.

After traversing the sensor 2104a or bypass 2104b section of pipe the system water 2106 re-joins the main pipe 2104. The sensor may be supplied with such a pipe section, in which case the main pipe 2104 may be provided with solderable joints, screw threads, etc. for connecting into a main water system.

In any case, the purpose of this arrangement is to provide a means for installing the sensor and for replacing the metal sample 2112. For example such a pipe arrangement may be fitted to the water system, and the valves set to their second positions 2124 so that system water 2106 flows through the bypass section 2104b, and not through the sensor section 2104a. In some cases, no sensor 2300 need be fit to the sensor section during installation. For example, this pipe arrangement may be installed with a view to fitting a sensor at a later date. Since the system water flows through the bypass section 2104b there is no danger that the system water will leak out of a hole in the sensor section 2104a of the pipe, since there is no water flow through that section. In other cases, the sensor section 2104a may be provided with a cover for plugging the hole where the sensor 2300 is intended to be installed.

In any case, when a sensor is to be fit to the system, the valves are set to their second positions 2124 to isolate the sensor mounting hole. It may be necessary at this stage to drain the sensor section 2104a of any system water 2106 in the sensor section. In other examples, it may be desirable not to drain the sensor section 2104a, e.g. to avoid introducing air into the system, which can reduce pumping efficiency and increase the rate of corrosion. In any case, once the sensor section 2104a no longer contains water under pressure, any cap can be removed from the hole for mounting the sensor 2300 and the sensor 2300 slotted into place. The sensor can be sealed in place using any suitable means such as using O-rings and clamps, screw threads, etc. In some cases, solder or welding may be used, but this can make removal of the sensor 2300 e.g. for replacement of the metal sample 2112, difficult.

Once the sensor 2300 has been mounted to the sensor section 2104a, the system water can be directed back though the sensor section 2104a by setting both valves to their first positions 2122. This causes system water 2106 to flow past the metal sample 2112 and thereby provides an in-situ measurement of corrosion in the water system.

In case the metal sample 2112 needs replacing, for example if it corrodes entirely away, or simply to the point where it is difficult to gain any further information on the corrosion state of the sample 2112, a corresponding process can be followed:
1. Set both valves to their second positions 2124 to direct system water 2106 down the bypass pipe 2104b.
2. Remove the sensor 2300 from the sensor pipe 2104b.
3. Replace the metal sample 2112 (and/or clean the sensor 2300, perform maintenance, etc.)
4. Replace the sensor 2300 in the sensor section 2104a and seal the sensor 2300 in place.
5. Set both valves to their first positions 2122 to direct system water 2106 down the sensor pipe section 2104a.

The sample 2112 may be held to the sensor 2300 using a screw thread, which can help to press the seal 2118 to form a watertight fit.

Consider now FIG. 16. This shows a sensor arrangement 2400 similar to that shown in FIG. 13. Here, however, the housing 2102 does not contain the light source 2108 or the light sensor 2116. Instead, the light source 2108 and the light sensor 2116 are housed in a second housing 2126, separated from the housing 2102. A fibre optic cable 2128 transmits the light from the light source 2108 to the interior of the housing and, once the light has reflected from the metal sample 2112, another fibre optic cable transmits the light back to the light sensor 2116. This arrangement means that even in the event that system water leaks into the housing 2102, the electronic parts of the sensor 2400 are protected from damage due to system water 2106.

This design is sealed from the system water 2106 by double O-ring seals 2118. One seal is located in contact with the metal sample 2112 and the system water 2106. The second seal is located behind the transparent plate 2120. This means that even in the case where the system water has corroded through the entire thickness of the metal sample 2112, system water 2106 is prevented from entering the housing 2102 by the second seal 2118 forming a seal with the transparent plate 2120 and the housing 2102. It should be borne in mind that the Figures are only schematic. For example, in order to provide uniform light intensity on the second surface of the metal sample 2112, it may be preferable for the fibre optic cable 2128 which transmits light from the light source 2108 to the interior of the housing 2102 to occupy substantially all of the upper surface of the housing 2102, and the other fibre optic cable 2128 to take up less of the upper surface of the housing 2102, or to exit the housing 2102 from a different surface. In other words, the fibre optic cables 2128 may be differently sized relative to each other and the housings 2102, 2126 than they appear in FIG. 16, which is not to scale.

In FIG. 17, yet another sensor apparatus 2500 is shown, in this case using a lens 2130 to focus light on the second surface of the metal sample 2112. Similarly to the sensing apparatuses described above, the sensor 2500 in FIG. 17 has a housing 2102 containing a light source 2108 and a light sensor 2116. The light 2110 emitted from the light source 2108 is directed not directly towards the metal sample 2112, but instead to a lens 2130. The lens 2130 directs the emitted light 2110 towards the second planar surface of the metal sample 2112. The lens can be configured to redirect the emitted light 2110 in such a way as to provide a homogenous light intensity over the entire second surface of the metal sample 2112. In other words, the lens can be used to smooth out any inhomogeneities in the emitted light 2110 to ensure that the sensor detects approximately the same loss of reflected light intensity irrespective of the location at which corrosion occurs on the metal sample 2112.

The light output 2132 from lens 2130 is directed to the metal sample 2112, whereupon it is reflected 2132 and enters the lens 2130 again (in some cases, there may be a second lens for receiving the reflected light). The lens 2130 directs the light towards the light sensor 2116. This can help to ensure that as much of the reflected light 2114 as possible is directed to the light sensor 2116, thereby improving the sensitivity of the sensor 2500 to small changes in reflectivity. In some cases, the lens 2130 may be replaced with one or more prisms, fibre optic arrangements, mirrors, etc. or a combination of these to achieve the same effect.

Additionally, the sensing apparatus 2500 has a baffle 2134 to block direct transmission of light 2110 emitted from the light source 2108 to the light sensor 2116. This reduces the occurrence of false negatives, as it prevents situations in which the reflected light 2114 is reduced due to corrosion, but this is not detected by the light sensor 2116 because the signal is swamped by directly transmitted light.

In some cases, it may be advantageous to combine the features shown in FIGS. 16 and 17, for example to provide a sensor with both a lens 2130 and fibre optic cables 2128 running to a second housing 2126. As noted above the fibre optic cable 2128 transmitting light to the housing 2102 should be relatively large to ensure that the light received by second surface of the metal sample 2112, is broadly uniform. An alternative way to achieve this is to mount a lens 2130 inside the housing 2102, as shown in FIG. 17, to provide a more even distribution of light emitted by the fibre optic cable 2128 onto the second surface of the metal sample 2112.

In FIG. 18, a further example of a sensing apparatus 2600 is shown. This example has a light source 2108 which is housed in a mounting 2140 attached to the internal walls of the housing 2102. The mounting 2140 has a recess housing the light source 2108, so that the mounting 2140 operates a little like the baffle 2134 shown in FIG. 17.

The light source 2108 in FIG. 18 is arranged to emit light into a fibre-optic bundle 2136. A first portion 2136a of the fibre optic bundle is directed towards a reference sensor 2138, the reference sensor also being housed in the mounting 2140. The first portion 2136a may be only a single fibre of the fibre-optic bundle 2136, or it may be a plurality of fibres. The first portion 2136a directs a portion of the light emitted by the light source 2108 to the reference sensor 2138. A second portion 2136b of the fibre optic bundle (in this case, the entire remainder of the bundle 2136) transmits the light towards the metal sample 2112. In other words, the end of second portion 2136b of the bundle 2136 which directs light towards the metal sample 2112 acts a little like the light source 2108 in other examples, in the sense that the second portion 2136b of the bundle emits light 2110 towards the metal sample 2112. This light is reflected and received and the intensity interpreted in the manner described above.

The reference sensor 2138 receives light directly from the light source 2108, so detects any variations in the emitted light intensity and/or spectral range without the emitted light 2110 being affected by environmental factors, such as the change in reflectivity of the metal sample 2112. This means that a drop in intensity of the reflected light 2114 detected by the light sensor 2116 can be correlated with the intensity measured by the reference sensor 2138. Where the intensity of the emitted light 2110 drops, the reference sensor 2138 and the light sensor 2116 will both detect a drop of approximately the same magnitude as each other, indicating a false positive which can be discounted. In cases where the light sensor 2116 detects a change in the intensity of reflected light 2114 which does not correlate with a drop in emitted light 2110 detected by the reference sensor 2138, or where the light sensor 2116 detects a drop larger than would be expected for a corresponding drop detected by the reference sensor 2138, then the event can be logged as representing corrosion.

FIG. 19 shows another example of a sensing apparatus 2700 having a reference sensor 2138. In this case, however, rather than using a fibre-optic bundle, the reference sensor 2138 is provided with a reference beam of light 2110a by virtue of a beam splitter 2142. As before, the light source 2108 emits a light beam 2110. This is directed towards the beam splitter 2142 which splits the emitted light beam 2110 into two beams 2110a and 2110b. The first of these beams 2110a is directed to the reference sensor 2138 and the reference sensor 2138 operates in much the same manner as discussed above in relation to FIG. 18. The second beam 2110b is directed towards the second surface of the metal sample 2112 and the beam is then reflected as a reflected beam 2114 and enters the light sensor 2116 in the manner set out above. Similarly to the situation described in respect of FIG. 18, the use of a reference sensor 2138 allows variations in the intensity and/or spectral range of the emitted light 2110 to be accounted for, so improving the reliability of measurements provided by the sensing apparatus 2700.

The beam splitter 2142 can be selected so that it splits any proportion of the light to the reference sensor 2138 as desired. Typically, only a small proportion of light (e.g. no more than 20%) should be diverted for reference sampling, so that the actual measurement is performed with a reasonable intensity of light. Naturally, this reduced absolute magnitude of the reference measurement can be adjusted to account for the smaller proportion of light being received by the reference sensor 2138.

Consider now FIG. 20 which shows a flow chart 2800 describing the operation of the method as set out herein. The method starts at step 2150 where a metal sample is mounted in a water system, the metal sample having a uniform thickness and including a first planar surface and a second planar surface opposite the first planar surface, wherein the first planar surface is arranged in contact with water of the water system. As noted above, this exposes only one surface of the metal sample to the system water, and consequently allows a determination of the extent and sometimes the type of corrosion by monitoring corrosion which penetrates throughout the uniform thickness of the metal sample.

The method continues at step 2152 in which light is emitted towards the second planar surface of the metal sample. As noted above, a light source may be supplied to emit the light, and is arranged to direct the emitted light towards the second planar surface. The light source may be adjustable in the sense that the intensity or spectral composition of the emitted light may be adjusted or controlled as part of the method. Indeed, the light source may be controlled in the sense that it emits no light most of the time, and is switched on periodically to emit light and provide a reading.

Next, at step 2154, the light reflected by the second planar surface of the metal sample is received, for example at a light sensor. The light reflects from the second planar surface in a known way, for example changing intensity and/or spectral range in a known way in response to an entirely clean surface, and changing intensity and/or spectral range in a known way, different to the first known way, in response to tarnishing or other signs of corrosion. The received light is analysed by factoring in the difference between the known composition of reflected light from a clean surface and the known composition of reflected light from a tarnished and/or otherwise corroded surface to arrive at an indication that the surface is showing signs of corrosion. Optionally, the sensor can determine the extent and/or type of corrosion. The sensor can be configured to synchronise with the light source to only detect reflected light at times when the light source is on, thereby reducing power consumption. Indeed, the signals from the sensor can also be analysed only when the light source is on, in order that resources are not used analysing data when no light is supplied to the metal sample. In other examples, the spectral range and/or intensity of the sensor are/is selected to conform to the expected intensity and/or spectral range of light emitted by the light source and reflected from the metal sample. Optionally, as set out above, the method may make use of a reference sensor to normalise the measurement and help rule out false positive results.

Next, at step 2156 the system (e.g. the light sensor) generates a signal indicative of the intensity of the reflected light. This may be an analogue signal, e.g. where the magnitude of a current or voltage output by the sensor is representative of the received light intensity. In other cases, the output may be digitised, for example to encode light levels as a digital signal, optionally, wherein different wavelength bands are separately encoded to allow for a spectral analysis. In one example, this may include sending digitised intensity values for each of a red, green and blue (RGB) band, similarly to how digital images are stored. In the RGB system, it is common for the peak intensity of the bands to be located at wavelengths of approximately: Red: 650 nm; Green: 525 nm; Blue: 440 nm, for example. In other cases, different spectral bands may be used, as appropriate, including more than three bands, for example or extending beyond the visible range of the electromagnetic spectrum.

In step 2158 the intensity of the reflected light is correlated with corrosion of the metal sample. This step relates, as set out above, a change in light intensity with the onset or progression of corrosion in the metal sample. Such a determination can be used to e.g. provide an alert to a maintenance team that pipes of a certain thickness and made from a particular metal are likely to fail soon and should be replaced.

Modifications to the general method 2800 set out in FIG. 20 may be made, for example by using some of the features of the examples set out respect of the other Figures.

Consider now FIGS. 21A and 21B, which show a plan and side view respectively of a metal sample system 2900 for use in the sensors described herein. The sample system 2900 comprises a metal disc 2112, having a layer of corrosion-resistant material 2144 having an annular shape around the edges of the metal disc 2112. As shown in e.g. FIG. 13, where a metal sample 2112 is mounted on a sensing apparatus 2100, there is a portion at the edges where a seal 2118 contacts the metal sample 2112. It has been found that the presence of the seal disproportionately increases the rate of corrosion near to the point of contact between the seal 2118 and the metal sample 2112. The corrosion-resistant coating 2144 is positioned to align with the region where the seal 2118 contacts the metal sample 2112. The corrosion-resistant coating may be wider than the footprint of the seal 2118 to allow a user to position the sample 2112 and the seal 2118 with a reasonably low degree of accuracy. There is a balance between allowing a user to be inaccurate in their positioning of the sample and ensuring that a reasonable area of metal is exposed to the system water.

As shown in FIG. 21B, the metal sample 2112 may be supplied with a corresponding transparent disc 2120. This may, for example, be adhered to the metal disc (using a compatible and transparent adhesive), or it may be supplied loose for holding in place by the clamping action of the seals 2118. Indeed, since the metal sample systems 2900 are intended to be replaceable, once a sensing apparatus has been sold, users may find it useful to buy replacement metal sample systems only. Since the transparent element 2120 does not usually become corroded or damaged, the metal sample 2112 with its corrosion-resistant coating 2144 may be supplied as a separate element that is the metal sample 2112 may be supplied without a transparent element 2120.

As an example, the metal disc 2112 may be formed from a metal representative of metals in the water system into which the metal disc 2112 is to be mounted, for example, carbon steel, aluminium, brass, copper, stainless steel, etc. The disc 2112 may be approximately 10 to 24 mm in diameter, and the corrosion-resistant coating 2144 may extend inwardly from the edge by approximately 3 mm. The metal disc 2112 may be supplied in various thicknesses, for example 0.025 mm, 0.05 mm, 0.075 mm, 0.01 mm, etc.

Turning to FIG. 22, which shows an arrangement for multiple metal samples 2112 in a sensing apparatus 2200. Here a plurality of metal samples 2112 is arranged in a grid. Horizontal rows are all formed from the same metal, and are shown separated by dashed lines simply to guide the eye. A first set of metal samples 2112a is formed from a first metal representative of metals in the system, for example copper. A second set of metal samples 2112b is formed from a second metal representative of metals in the system, for example brass. A third set of metal samples 2112c is formed from a third metal representative of metals in the system, for example aluminium. A fourth set of metal samples 2112d is formed from a fourth metal representative of metals in the system, for example stainless steel. An arrow (A) shows the direction in which thickness increases for each set of metal samples 2112a-2112d. In some cases, for example, the first metal sample 2112 (the leftmost sample) in each set of samples 2112a-2112d may have the same thickness, e.g. 0.025 mm. The next samples moving to the right in the direction of arrow (A) may have respectively thicknesses of 0.05 mm, 0.075 mm and 0.1 mm. In other examples, the metals in each set of metal samples 2112a-2112d may have different thicknesses, for example based on the likelihood of corrosion of that metal type, the typical thicknesses of components made from that metal, and the degree of accuracy required for that metal. In general, metals which corrode quicker may have thicker metal sample 2112 sizes than metals which corrode slower. Similarly, where components tend to be made thicker (or tend to be able to withstand severe pitting), thicker metal samples 2112 may be chosen. As noted above, the difference in thickness between adjacent metal samples 2112 is related to the accuracy with which the pitting depth can be determined. Therefore, the progression of thickness of samples 2112 can be selected to provide the desired resolution for that metal.

While four thicknesses of each type of metal are shown in the Figure, some cases may have more or fewer samples 2112. Indeed, in some case each metal type may have a different number of thicknesses, depending on the information a user wishes to obtain. While four metal types are shown in this example, different examples may provide more or fewer metal types.

While not shown here, the sensing apparatus has the features described above for making the measurement, such as at least one light source 2108, at least one light sensor 2116, etc. Multiplexing may be used as described above to provide fewer light sources 2108 and/or light sensors 2116 than the number of metal samples 2112 (i.e. fewer than 16 light sources 2108 and/or sensors 2116 in the example shown in FIG. 22).

In any of the above examples of sensing apparatuses, internal walls of the housing may have diffuse reflective inner surfaces for providing an optical integrating cavity. Optical integrating cavities are chosen so that all light emitted by the light source eventually reaches the light sensor. They have the effect of smoothing out the light intensity in the interior of the housing so that each portion receives approximately the same light intensity. This prevents inhomogeneities in the angular distribution of light emitted from the light source from causing different parts of the sample to be illuminated with different intensities of light and ensures that the background light levels are highly consistent. Such a situation could lead to different parts of the sample showing different effects when corrosion occurs, if the inhomogeneities are severe enough. Of course, providing a highly homogeneous light source is another solution to this. Where the internal walls of the cavity are said to be diffuse reflective surfaces, this does not necessarily apply to the second planar surface of the metal sample, and certainly does not apply to the transparent element, where such an element is present.

It will be appreciated from the above description above that many features of the different examples are interchangeable with one another. The disclosure extends to further examples comprising features from different examples combined together in ways not specifically mentioned. Indeed, there are many features presented in the above examples and it will be apparent to the skilled person that these may be advantageously combined with one another. Examples of features which are combinable with other features in this way include: the baffle 2134 shown in FIG. 17; the use of multiple metal samples 2112 in a single sensing apparatus as shown in FIG. 14; the use of multiplexing with multiple samples to reduce the number of light sources and/or sensors in an apparatus; the use of the double O-ring seal arrangement of FIGS. 16 to 19 for improving the seal; the use of fibre optic cables 2128 as in FIG. 16 to mount the light source 2108 and light sensor 2116 in a second housing 2126; and the use of a lens 2130 as shown in FIG. 17.

1-105. (canceled)

106. An optical sensing apparatus for mounting in a water system and for monitoring corrosion in the water system, comprising:
a metal sample having a uniform thickness and a first planar surface and a second planar surface opposite the first planar surface, wherein the first planar surface is arranged to be in contact with water within the water system;
a light source configured to emit light towards the second planar surface of the metal sample; and
a light sensor configured to receive light reflected by the second planar surface of the metal sample, and output a signal indicative of the intensity of the reflected light.

107. An optical sensing apparatus according to claim 106, further comprising a transparent element disposed at least partly between the light source and the metal sample.

108. An optical sensing apparatus according to claim 107, wherein the transparent element has a third planar surface arranged adjacent to the second planar surface of the metal sample.

109. An optical sensing apparatus according to claim 106, further comprising a seal for protecting the second planar surface from water in the water system.

110. An optical sensing apparatus according to claim 109, wherein the seal is located adjacent to the first planar surface and wherein the metal sample is provided with a corrosion resistant coating on the first planar surface in the vicinity of the seal.

111. (canceled)

112. An optical sensing apparatus according to claim 106, wherein the metal sample is a film having a thickness of 1 mm or less.

113-118. (canceled)

119. An optical sensing apparatus according to claim 106 wherein the or each metal sample is replaceable.

120. An optical sensing apparatus according to claim 106, further comprising a processor configured to receive the signal indicative of the intensity of the reflected light from the light sensor.

121. (canceled)

122. An optical sensing apparatus according to claim 120, wherein the processor is configured to relate the signal indicative of the intensity of the reflected light to corrosion of the metal sample, and wherein the light sensor is configured to output a plurality of signals indicative of the intensity of the reflected light over time and wherein the processor is configured to determine a rate of corrosion of the metal sample from the plurality of signals indicative of the intensity of the reflected light over time received from the light sensor.

123. (canceled)

124. An optical sensing apparatus according to claim 120, wherein the processor is configured to receive a plurality of signals indicative of an amount or a rate of corrosion of a corresponding plurality of metal samples; and wherein the plurality of metal samples are formed from the same metal as one another, wherein each of the plurality of metal samples has a different thickness and wherein the processor is configured to determine a range of maximum pinhole corrosion depths in the water system from the plurality of received signals.

125. An optical sensing apparatus according to claim 106, further comprising an optical element configured to direct light emitted by the light source towards the second planar surface of the metal sample, and/or configured to direct the reflected light towards the light sensor.

126-133. (canceled)

134. A method of monitoring corrosion in a water system, the method comprising:
mounting a metal sample in a water system, the metal sample having a uniform thickness and including a first planar surface and a second planar surface opposite the first planar surface, wherein the first planar surface is arranged in contact with water of the water system;
emitting light towards the second planar surface of the metal sample;
receiving light reflected by the second planar surface of the metal sample at a light sensor;
generating a signal indicative of the intensity of the reflected light; and
correlating the intensity of the reflected light to corrosion of the metal sample.

135. A method according to claim 134, further comprising generating a plurality of signals indicative of the intensity of the reflected light over time, and determining a rate of corrosion from the plurality of intensities of the reflected light over time.

136-139. (canceled)

140. The method according to claim 134, further comprising a second light sensor for directly sampling the light emitted from the light source to provide a reference value, wherein correlating the intensity of the reflected light to corrosion of the metal sample includes comparing the reference value to the received light reflected from the second planar surface.

141-147. (canceled)

148. The method according to claim 134, further comprising controlling one or more of:
power;
intensity; and/or
spectral weight
of the emitted light.

149. A sample for use in an optical sensor for monitoring corrosion in a water system, the sample comprising:
a metal element having:
a first surface for exposure to the water of the water system; and
a second surface opposite the first surface for receiving and reflecting light; wherein
a portion of the first surface is provided with a corrosion-resistant coating for providing a location for forming a seal between the sample and the sensor.

150. The sample of claim 149, wherein the metal element is planar and/or has a uniform thickness.

151. The sample of claim 149, wherein the corrosion-resistant coating is applied to the edges of the metal element.

152. The sample of claim 151, wherein the corrosion-resistant coating extends between 0.5 mm and 5 mm inward from the edge of the first surface.

153-155. (canceled)

156. The sample of claim 149, wherein the metal element is formed from stainless steel, copper, brass, aluminium, or other materials representative of metals in the water system.

157-159. (canceled)
